(19) United States
(12) Patent Application Publication
(10) Pub. No.: US 2019/0085394 A1
(43) Pub. Date: Mar. 21, 2019

(54) REFINING DIAGNOSIS AND TREATMENT OF COMPLEX MULTI-SYMPTOM NEUROLOGICAL DISORDERS

(71) Applicant: PARKINSON'S INSTITUTE, Sunnyvale, CA (US)

(72) Inventors: J. William Langston, Sunnyvale, CA (US); Birgitt SCHÜLE, Sunnyvale, CA (US); Linda REES, Sunnyvale, CA (US); R. Jeremy NICHOLS, Sunnyvale, CA (US); Carrolee BARLOW, Sunnyvale, CA (US)

(21) Appl. No.: 16/061,649

(22) PCT Filed: Dec. 14, 2016
(86) PCT No.: PCT/US16/66683

§ 371 Date: Jun. 12, 2018

Related U.S. Application Data
(60) Provisional application No. 62/267,224, filed on Dec. 14, 2015.

(51) Int. Cl. C12Q 1/6883; C12Q 1/686; C12Q 1/6806; G06F 19/12; G06F 19/24; G06F 19/20

(52) U.S. Cl. CPC C12Q 1/6883; C12Q 1/686; G06F 19/20; G06F 19/12; G06F 19/24; C12Q 1/6806

(57) ABSTRACT

The present invention provides methods for defining a disease or condition with a wide range of etiologies based on clustering of genes belong to the same biological pathway and observed frequencies or prevalence of various patient factors assessed in a given patient population, including motor and non-motor symptoms, neuropathology, age of onset, genetics, and involvement of peripheral autonomic system, including the enteric nervous system of the gastrointestinal (GI) system. Also disclosed herein are methods and kits for early diagnosis and treatment of subjects for multisystem Lewy body disease (MLBD), including Parkinson's disease, and/or one or more GI conditions that indicate a propensity for MLBD.

### CROSS-REFERENCE TO RELATED APPLICATIONS

This application claims the benefit of U.S. Provisional Application No. 62/267,224, filed Dec. 14, 2015, which application is incorporated herein by reference in its entirety.

### BACKGROUND

Over the last half century, new discoveries and insights into Parkinson's disease (clinically defined as bradykinesia, resting tremor, and rigidity with pathology of loss of dopaminergic nigral neurons and the presence of intraneuronal inclusions known as Lewy bodies) have been plentiful, and in some instances revolutionary. These range from the recognition of the importance of the substantia nigra in the 1950s (1) to the observations of a nigrostriatal dopamine deficiency as the main cause of symptoms and signs of the disease. (1-3) This, in turn, led to the identification, in 1968, of the first effective treatment (L-dopa) for the motor symptoms of the disease. (2) In 1983, the parkinsonogenic neurotoxin 1-methyl-4-phenyl-1,2,3,6-tetrahydropyridine (MPTP) (3) was discovered, leading to the creation of the first good animal model for the disease and stimulating a renaissance of interest in environmental toxins as potential causes for it. More recently, the first monogenic form of Lewy body parkinsonism similar to Parkinson's disease, caused by the NM_000345.3(SNCA):c.157G>A (p.Ala53Thr) mutation in the gene encoding a-synuclein (4), ignited an explosion of interest and discoveries in the genetics of Parkinson's disease. These genetic data have provided important new biological insights (5), just as the discovery of MPTP did. Notwithstanding all of these developments, however, nearly 50 years after its discovery, L-dopa still remains the most effective drug for Parkinson's disease, allowing management of some symptoms but still far from an ideal therapy.

### SUMMARY

The present disclosure provides a novel approach for classifying or parsing out complex diseases or conditions with a wide range of etiologies into subclasses or subtypes based on a common biological pathway or mechanism of associated genes.

In one aspect, disclosed herein is a method of distinguishing a disease from multiple diseases associated with similar symptoms comprising (a) building a tissue bank derived from samples of a plurality of subjects displaying at least one symptom of the similar symptoms; (b) characterizing each of the samples by performing at least one of sequencing a nucleic acid, quantifying a nucleic acid or a protein, detecting a histopathological abnormality, and detecting a protein-protein interaction; (c) building a data bank derived from assessing the subjects, wherein the data comprises information selected from at least one of: the at least one symptom, age of disease onset, and environmental circumstances of the subjects; (d) identifying a sub-group in the plurality of subjects, wherein the sub-group possesses at least one similar tissue characteristic and at least one data characteristic; and (e) determining whether the sub-group has the disease. The disease may be a neurological disease or condition, a neurodegenerative disease or condition, a neuromuscular disease or condition, a liver disease or condition, a gastrointestinal disease or condition, a metabolic disease or condition, or an autoimmune disease or condition. The plurality of subjects may comprise at least ten subjects, at least fifty subjects, or at least a hundred subjects. The sequencing may comprise sequencing at least a portion of a gene or gene transcript known to harbor a genetic mutation. The genetic mutation may be associated with at least one disease of the multiple diseases. The portion of the gene may be at least about ten nucleotides. The building the tissue bank may comprise freezing the samples, and the samples may comprise a fluid sample selected from a blood sample, a saliva sample, a urine sample, a spinal fluid sample, a plasma sample, or a lymphatic fluid sample. In addition, the samples may comprise tissue samples, biopsy samples, cadaver samples, or whole cells. The quantifying the nucleic acid may comprise quantitative PCR. The detecting the histopathological abnormality may comprise contacting the sample with a stain or a detectable tag-conjugated antibody. The building the data bank may comprise administering a questionnaire to the subjects. In some embodiments of the method, at least one of the proteins involved in the protein-protein interaction are known to be involved in a biological pathway implicated in any one of the multiple diseases.

Also disclosed herein is a method of distinguishing a first disease from a second disease, wherein the first disease and the second disease are associated with similar symptoms comprising (a) collecting biological samples from a plurality of subjects displaying at least one symptom of the similar symptoms; (b) sequencing a nucleic acid in the biological samples to identify a subgroup of the plurality of subjects expressing a genetic mutation; (c) recording at least one symptom experienced by the plurality of subjects; (d) identifying a sub-group in the plurality of subjects, wherein the sub-group possesses the genetic mutation and displays the at least one symptom; and (e) determining the sub-group has the disease. The method may further comprise assessing a test subject for having the disease comprising (a) collecting a biological sample from the test subject; (b) sequencing or quantifying a nucleic acid or a peptide in the biological sample; (c) observing at least one symptom experienced by the test subject; and (d) the subject as having the disease when the subject possesses the genetic mutation and displays the at least one symptom. In addition, the method may comprise treating the test subject with an agent specific for the disease.

In one embodiment, a method of defining or parsing out complex diseases or conditions with a wide range of etiologies into subclasses or subtypes based on a common biological pathway or mechanism of associated genes, involving the steps of collecting patient data, analyzing two or more factors of the following factors to determine an observed frequency of each factor in a given patient population, including data on family history, genetic mutation, motor symptom, non-motor symptom, neuropathology, age of onset, and symptoms involving peripheral autonomic system; mapping observed frequencies of the various factors to determine a cluster of the analyzed factors, linking the cluster of analyzed factors to one or more genes to determine the genes underlying a subclass corresponding to the observed frequencies of the analyzed factors; analyzing protein-protein interactions of the genes linked to the subclass to validate a common biological pathway or mechanism; and defining the subclass as a distinct disease or condition based on the underlying mechanism identified. In some cases, the complex diseases or conditions comprise Parkinson's disease and parkinsonian diseases or conditions. In other cases, the complex disease or conditions comprise dementia, Alzheimer's disease, or a cancer. Such method can be applied to identify the subclass of multisystem Lewy body disease (MLBD). As disclosed herein, peripheral autonomic system involves assessing the gastrointestinal (GI) system for dysfunction and/or cardiac abnormality. In some embodiments, motor symptoms include one or more of muscle rigidity, tremor, gait and postural abnormalities, a slowing of physical movement (bradykinesia), and a loss of physical movement (akinesia), while non-motor symptoms comprise symptoms measurable by a cardiac scan or symptoms relating to gastrointestinal (GI) motility. In some cases, neuropathology comprises formation of Lewy bodies in a sample of nerve cells extracted from a subject. In some embodiments, observed frequencies or prevalence of analyzed factors in a given patient population can involve mapping frequencies using distance matrices or plotting out Euclidean distances to visualize clustering of certain factors, such as genes. Gene mutations involved in MLBD or parkinsonian diseases can include one or more mutations in LRRK2, GBA, SNCA, VPS35, DJ-1, PINK1, PARK2, DNAJ13C, and any combination thereof. In some embodiments, three genes are predominantly associated with MLBD or Parkinson's disease, such as LRRK2, GBA, SNCA, and any combination thereof.

Also disclosed herein is a method of characterizing a complex disease or condition comprising: identifying one or more allelic variants in one or more genes associated with the disease or condition; determining clinical pathology or symptoms associated with each allelic variant in a patient population; grouping the genes with allelic variants based on the degree of overlap between their clinical pathology or symptoms and a standard set of clinical pathology or symptoms; determining proteins and/or genes that interact with each group of genes with allelic variants to construct protein interaction networks that inform the molecular mechanism or cellular process affected by the allelic variants; and characterizing said disease or condition based on the molecular mechanism or cellular process associated with one or more allelic variants. In such cases, the complex disease or condition can be multisystem Lewy body disease, Parkinson's disease, or Parkinsonism; wherein one or more allelic variants is selected from the group consisting of: LRRK2, GBA, SNCA, VPS35, DJ-1, PINK1, PARK2, DNAJ13C, and any combination thereof; and wherein the standard set of clinical pathology or symptoms refers to Parkinson's disease. In some cases, group of genes used to construct protein interaction networks for understanding the underlying pathway or mechanism include any one of the following groups: LRRK2, GBA, and SNCA; LRRK2 and SNCA; LRRK2 and GBA; or GBA and SNCA.

In some cases, a method of treating a disease or condition involves diagnosing a subject, which can be a human or a mammalian, using any of the methods above. In some instances, the subject is diagnosed with MLBD. In some cases, the method involves administering one or more of the following therapeutic agents to the subject: L-dopa, monoamine oxidase B inhibitor, dopamine agonist, catechol-O-methyltransferase inhibitor, anticholinergic, amantadine, or any combination thereof.

Another method disclosed herein involves treating a disease or condition, which can be MLBD, Parkinson's disease, or parkinsonian, comprising the steps of: obtaining a genetic sample from a patient; sequencing the genetic sample for one or genes associated with the disease or condition; identifying one or more allelic variants in the genes associated with the disease or condition; identifying proteins and/or genes that interact with the genes associated with the disease or condition to determine the molecular mechanism or cellular process affected by the allelic variants; and administering a therapy or pharmaceutical agent directed to the molecular mechanism or cellular process affected by the allelic variants. In some cases the allelic variant is a gene selected from the group consisting of: LRRK2, GBA, SNCA, and any combination thereof. In other cases, one or more allelic variants is in: LRRK2, GBA, and SNCA; LRRK2 and SNCA; LRRK2 and GBA; or GBA and SNCA. In some embodiments, the therapy or pharmaceutical agent includes L-dopa, monoamine oxidase B inhibitor, dopamine agonist, catechol-O-methyltransferase inhibitor, anticholinergic, amantadine, or any combination thereof.

Also described herein is a method of screening a subject for a neurological condition, comprising: measuring a GI condition using one or more of the following methods: an esophageal and/or anorectal manometry, a G-Tech monitoring device, a GI Symptom Relief Scale (GSRS), a Gastroparesis Cardinal Symptom Index (GCSI), a UPSIT, a Hoehn Yahr, UPDRS motor scale, a wireless motility capsule, and combinations thereof; comparing the GI measurement against an observed frequency of the GI condition in a population of patients diagnosed as having the neurological condition; assessing one or more of the following factors to further validate a diagnosis of the neurological condition: genetic mutation, clinical symptom, neuropathology, and diagnosing the subject as having the neurological condition if the GI measurement and the one or more factors correspond to high observed frequencies in the population of patients diagnosed as having the neurological condition.

In some embodiments, a method of screening a therapy for therapeutic efficacy towards a neurological condition and/or symptoms thereof involves performing an assessment of a GI condition; assigning a quantitative value to the GI condition based on the assessment; comparing said quantitative value to a value range predetermined to be indicative of Parkinson's disease or Parkinson's-like disease; and identifying said subject as suffering from or prone to Parkinson's disease or Parkinson's-like disease if said quantitative value falls in said value range. In some embodiments, the assessment comprises performing a procedure selected from an esophageal manometry and an anorectal manometry. In other embodiments, the assessment comprises administering a questionnaire, wherein the test or survey comprises questions regarding the GI symptom, which can be a survey, a test, a scale, and an index. In some cases, the test is selected from a University of Pennsylvania Smell Identification Test and a modification thereof. In other cases, the scale is selected from a GI Symptom Relief Scale, a Hoehn and Yahr Scale, a UPDRS scale and modifications thereof. In some embodiments, the assessment comprises using a device selected from a wireless motility capsule, a G-Tech monitoring device, and modifications thereof. In some cases, the method includes analyzing a biological sample from the subject, wherein the biological sample is selected from a blood sample, a urine sample, a saliva sample, a skin sample, a hair sample and a fecal sample. In some cases, the method involves obtaining a biological sample from the subject, which can be a blood draw, a GI biopsy, and a surgical resection. The analysis of the biological sample comprises analyzing an expression level of a gene or mutation thereof and/or an amount of protein encoded by the gene or mutation thereof, wherein the gene can be selected from parkin, leucine-rich repeat kinase 2, and alpha-synuclein. The analysis of the biological sample includes analyzing a degree of neuronal loss in the biological sample. In some embodiments, the neurological condition is MLBD or Parkinson's Disease.

A kit for carrying out any of the methods described herein is also contemplated, wherein the kit comprises devices and/or questionnaires for assessing GI symptoms selected from tools for performing an esophageal and/or anorectal manometry, wireless motility capsule, a G-Tech monitoring device, a GI Symptom Relief Scale (GSRS), a Gastroparesis Cardinal Symptom Index (GCSI), a UPSIT, a Hoehn Yahr Scale, a UPDRS scale and combinations thereof, tools for collecting a tissue/fluid sample, devices and/or reagents for nucleic acid and/or protein purification and oligonucleotides and/or antibodies for nucleic acid and/or protein detection. In some cases, the kit also includes oligonucleotides and/or antibodies may be specific for nucleic acids and/or proteins comprising genetic mutations associated Parkinson's disease, such as a mutation in parkin, alpha-synuclein, and LRRK2.

In some embodiments, a method of treating MLBD in a subject comprises: obtaining a sample of enteric nerves from a subject for ex vivo experiments and testing; assaying the sample for a genetic mutation or an abnormality in one or more genes selected from the group consisting of: LRRK2, SNCA, GBA, and any combination thereof; comparing the genetic mutation or abnormality of the sample of enteric nerves to genetic mutations or abnormalities of the same genes associated with MLBD and/or PD; using the genetic mutation or abnormality of step (c) to select one or more therapeutic agents that target the one or more genes; applying the one or more therapeutic agents to the sample of enteric nerves ex vivo to predict their efficacy on cells of the central nervous system; and treating the subject's neurological condition based on the efficacy of the one or more therapeutic agents on the sample of enteric nerves.

In other aspects, a method of screening a neuroprotective agent comprises obtaining a sample of a subject's enteric nerve cells; applying one or more neuroprotective agents to the sample in vitro to determine an effect on one or more biomarkers present in both enteric nerve cells and central nervous system cells, wherein the biomarkers correlate with MLBD and/or PD; optionally, validating the effect by applying the neuroprotective agents to neuronal cells in vitro; and identifying one or more neuroprotective agents with a therapeutic effect based on effect on enteric nerve cells and/or neuronal cells in vitro.

In some embodiments, a method for identifying a prioritized set of genes that facilitate diagnosis or treatment of a disease, comprising: isolating tissue samples from human subjects with genetically causal forms of the disease; comparing genetic and allelic variants of the disease based on different phenotypes or presentation of the disease; and prioritizing genes causing the disease based on degree of overlap in common protein interactions among products of genes associated with different phenotypes or presentation of the disease resulting in the prioritized set of genes, wherein the disease is Parkinson's disease (PD), wherein data on peripheral autonomic system are used to differentiate Multisystem Lewy body disease (MLBD) and non-Lewy body parkinsonian or Parkinson-like diseases, or wherein the phenotypes or presentation of the disease include idiopathic PD, Multisystem Lewy body disease (MLBD), mixed MLBD, and parkinsonism, or wherein protein interactions are MLBD protein interactions. In some cases, the prioritized set of genes closely associated with Parkinson's disease is selected from a group consisting of LRRK2, SNCA, GBA, and a combination thereof, which can be targets for identifying disease or modifying agents, wherein the prioritized set of genes containing human mutations is incorporated in a transgenic or animal model for studying the disease. In some cases, the prioritized set of genes containing human mutations is incorporated in a cell line or ex vivo model. In some cases, the prioritized set of genes containing human mutations is used to screen patients for a clinical study. In other cases, the prioritized set of genes containing human mutations is used to design gene-environment studies.

Also disclosed herein is a method for ensuring clinically collected data can be used for therapeutic decisions or research without increasing noise and confusion in large data collections associated with Parkinson's disease by prioritizing genetic forms of Parkinson's Disease as multisystem Lewy body disease, comprising: analyzing pathological diagnosis of genetic subtypes of Parkinson's disease based on common mutational etiology, differing outcomes from varying allelic, and disease-associated variants; delineating parkinsonian disorders into subclasses on the basis of molecular mechanisms with well-characterized outcome expectations; and prioritizing genetic forms of Parkinson's disease as multisystem Lewy body disease based on the analysis of the pathological diagnosis and the delineation of the parkinsonian disorders into subclasses.

In some cases, a method of treating a neurological condition in a subject comprises determining a risk factor for a neurological condition in a subject, performing an assessment of one or more GI condition in said subject; conducting a treatment protocol if the subject has a risk factor for a neurological condition and has one or more GI conditions.

In some instances, a method of treating a neurological condition in a subject comprises administering a diagnostic test in a subject to determine whether the subject has small intestinal bacterial overgrowth; treating the subject if the subject has small intestinal bacterial overgrowth. In some embodiments, the diagnostic test can be a wireless motility capsule (WMC) or a breath test.

In some embodiments, a method of treating a neurological condition in a subject, comprises administering a muscle-specific agent to a subject; and performing physical therapy by said subject, wherein the muscle agent can be botulinum toxin.

In other embodiments, a method of screening a subject for a multisystem Lewy body disease (MLBD) comprising performing an assessment of two or more of the following factors of a subject, such as motor symptoms; mutation in one or more genes selected from the group consisting of: LRRK2, GBA, SNCA; neuropathology; and abnormality in peripheral autonomic system; assigning a quantitative score to the assessed factors based on prevalence of the assessed factors in MLBD patients; comparing the quantitative score to a predetermined range indicative of MLBD; and identifying the subject as suffering from or prone to MLBD if the quantitative score falls in the range, wherein the motor symptoms assessed comprise bradykinesia, tremor, postural instability, or rigidity. The assessment of abnormality in peripheral autonomic system can be performed using a cardiac MIBG scintigraphy scan or involve assessing the subject's gastrointestinal (GI) motility using various GI motility methods, including esophageal manometry, anorectal manometry, wireless motility capsule, or GI symptom questionnaires. In some cases, abnormality in peripheral autonomic system comprises assessing a sample of the subject's enteric nervous system for a genetic mutation in one or more of LRRK2, GBA, and SNCA, wherein assessment of neuropathology comprises detecting alpha-synuclein positive Lewy bodies or Lewy neurites in nerve cells of the subject, wherein the MLBD is Parkinson's disease. In some cases, the method can further comprise administering a neuroprotective agent to the subject having a quantitative score indicative of MLBD, or testing efficacy of the neuroprotective agent by measuring a change in the quantitative score based on assessment of one or more of the following factors after administering the neuroprotective agent: motor symptoms; neuropathology; abnormality in peripheral autonomic system; or any combination thereof. In some cases, a change in the quantitative score is measured over time.

In other embodiments, a method of early diagnosis of MLBD in a subject comprises performing an assessment of a subject's peripheral autonomic system dysfunction; assigning a quantitative score to the assessment of the subject's peripheral autonomic system dysfunction based on prevalence of the dysfunction in MLBD patients; comparing the quantitative score to a predetermined range indicative of risk of developing MLBD; and identifying the subject as suffering from or prone to MLBD if the quantitative score falls in the predetermined range, wherein one or more of the following factors of the subject is assessed: motor symptoms; mutation in one or more genes selected from the group consisting of: LRRK2, GBA, and SNCA; neuropathology; or any combination thereof; and assigning a quantitative score for the assessed factors based on prevalence in MLBD patients. In some cases, peripheral autonomic system dysfunction comprises cardiac denervation or gastrointestinal (GI) dysfunction, or can further comprise administering a neuroprotective agent to treat the subject whose quantitative score is above a threshold as compared to a control. In some cases, the method can further comprise administering a therapeutic agent to treat the subject's GI dysfunction in combination with the neuroprotective agent, wherein assessing the subject's GI dysfunction comprises assessing the subject's enteric nervous system for a genetic mutation in one or more of genes selected from the group consisting of: LRRK2, GBA, SNCA, and any combination thereof. In other embodiments, the method can further comprise assessing the subject's enteric nervous system for presence of alpha-synuclein positive Lewy bodies or Lewy neurites.

In some embodiments, a method of diagnosing a subject for Parkinson's disease comprises: performing an assessment of a gastrointestinal (GI) condition using one or more of the following methods: esophageal manometry, anorectal manometry, wireless motility capsule, GI symptom questionnaires, or any combination thereof; assigning a quantitative score to the GI condition assessed based on prevalence of the condition in Parkinson's disease patients; and comparing said quantitative score to a predetermined range indicative of risk of developing Parkinson's disease. In some cases, the method further comprises administering a neuroprotective agent to the subject whose quantitative score is above a threshold value, or obtaining a biopsy of the subject's enteric nervous system and testing said biopsy for a genetic mutation in one or more of genes selected from the group consisting of: LRRK2, GBA, SNCA, and any combination thereof, or obtaining a biopsy of the subject's enteric nervous system and testing said biopsy for presence of alpha-synuclein positive Lewy bodies or Lewy neurites. In some embodiments, a method of treating Parkinson's disease involves identifying a subject as having a risk of developing Parkinson's disease by performing the previous steps and obtaining a sample of the subject's enteric nervous system; determining the subject's responsiveness to a therapeutic agent by screening therapeutic agents using the sample of the subject's enteric nervous system; and treating the subject with one or more of the therapeutic agents to which the subject's enteric nervous system is most responsive.

In other embodiments, a method of developing a neuroprotective factor comprises generating an enteric nerve cell line from a subject with a mutation in one or more of genes selected from the group consisting of: LRRK2, GBA, SNCA, VPS35, DJ-1, PINK1, PARK2, GCH1, ATXN2, and DNAJ13C; screening one or more therapeutic agents for therapeutic efficacy in the enteric nerve cell line; identifying therapeutic agents with therapeutic efficacy; and testing the therapeutic agents in a multisystem Lewy body disease model.

In some embodiments, a method of developing a neuroprotective factor involves generating an enteric nerve cell line from a subject with Parkinson's disease; screening one or more therapeutic agents for therapeutic efficacy in the enteric nerve cell line; identifying therapeutic agents with therapeutic efficacy; and testing the therapeutic agents in a multisystem Lewy body disease model, wherein the enteric nerve cell line includes alpha-synuclein positive Lewy bodies or Lewy neurites, and wherein the therapeutic efficacy refers to amelioration of such Lewy bodies or Lewy neurites.

In other embodiments, a method of diagnosing MLBD or PD comprises assessing a subject's GI motility using one or more of the following methods: esophageal manometry, anorectal manometry, wireless motility capsule, GI symptom questionnaires, a G-Tech monitoring device, a GI Symptom Relief Scale (GSRS), a Gastroparesis Cardinal Symptom Index (GCSI), a UPSIT, a Hoehn Yahr Scale, a UPDRS scale, or any combination thereof.

In some embodiments, a method of treating MLBD or Parkinson's disease comprises administering a neuroprotective agent to the subject diagnosed with MLBD or Parkinson's disease using any of the methods described herein. In such methods, the therapeutic agent can be one or more of the following: carbidopa, levodopa, dopamine agonist, MAO-B inhibitor, Catechol-O-methyltransferase (COMT) inhibitor, anticholinergics, amantadine, antibody, and any combination thereof. In some embodiments, methods described herein are used to diagnose or treat a subject with pre-motor symptoms of the disease, i.e., before progression to the brain.

In other embodiments, a method of screening neuroprotective agents having a therapeutic effect on enteric and central nervous system involves screening agents for efficacy or therapeutic effect using a cell line derived from enteric cells of a subject diagnosed with Parkinson's disease or MLBD.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE FIGURES

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an illustrative bubble graph of genetic forms of parkinsonism. To more readily visualize the relationship between various forms of parkinsonism and sporadic Parkinson's disease (referred to as MLBD), information in TABLE 1 (see also FIG. 10) (which is organized on the basis of genetics, clinical assessments and neuropathology) was used to calculate the Euclidean distance of the clinical manifestations of the gene, mutation or condition listed relative to sporadic Parkinson's disease (x axis). The y axis represents the age of onset of disease, and the size of each bubble represents the relative prevalence (see TABLE 6 and FIG. 15). The circle labeled “PD” refers to idiopathic PD where genotype is not known. Because there are varying degrees of robustness of neuropathological data, different shades were used to reflect this as follows: darkest shade indicates Lewy body pathology in all cases; medium shade indicates variable findings with the majority of cases showing Lewy body pathology; light shade indicates Lewy body pathology in only a few cases; lightest shade indicates that Lewy body pathology was not found but the data are sparse or incomplete, or no data were available. Data were plotted versus average age of onset for all variants. The size of each circle represents the relative frequency of the genetic forms. To aid in visualization, the radius of the circles are scaled relative to the most common form by subtracting the log 2 value of the observed prevalence from the log 2 value of the least-common form. These factors allowed for easier visualization of the relative frequency over the observed range. However, it should be noted that the scale is not linear with respect to the actual observed frequencies and the reader is referred to TABLE 1 (see also FIG. 10) for more detail. The area between the grey hashed lines indicates early-onset parkinsonism and below the bottom grey hash line, juvenile (<20y) parkinsonism.

FIG. 2 shows an exemplary list of protein interactions for genes with mutations that are known to cause parkinsonism but do not always manifest with the same neuropathological findings (LRRK2, GBA, SNCA, VPS35, DJ-1, PINK1, PARK2 and DNAJ13C), which can be found at http://www.thepi.org/scientific-resources/. There were multiple interactions with these proteins, but only a single interaction was found in common among the eight: human ubiquitin C. STRING DB and exported the protein interaction network for all human proteins were assessed. Protein interaction networks from this data for three groupings of genes are found in TABLE 1 (see also FIG. 10). HUGO terms in STRING DB were used to identify the gene products and interactors. Knowledge Explorer™ (available from IO Informatics) was used to visualize the protein interaction network from STRING DB for the gene products of interest (FIGS. 2-4). The list of proteins that interact with the highly validated MLBD associated genes LRRK2, GBA, SNCA are shown in FIG. 4 and can be found in TABLE 7 and FIG. 16.

FIG. 3 shows that when the protein interaction network search was limited to only genes that are associated with MLBD or possible MLBD (LRRK2, DNAJC13, GBA, and PINK1), only two common interacting proteins, UBC and Hsp70 (HSPA4), were found, in accordance with some embodiments.

FIG. 4 shows that interaction network for proteins encoded by genes best characterized to cause MLBD (LRRK2, SNCA and GBA), in accordance with some embodiments. There was an extensive overlap in the number of common interactions (more than 50, see TABLE 7 for a list of these interactions; see also FIG. 16).

FIG. 5 provides exemplary immunohistochemical images revealing expression of α-synuclei (green) and TuJ1 (green) in the enteric nervous system, in accordance with some embodiments. Human intestine showing alpha-synuclein (green), TuJ1 (neurons—red) and nuclei (blue) demonstrating alpha-synuclein is present in ENS neurons (merge=yellow). Panel A-A′: TuJ1 stained long sensory axons in the submucosal plexus (SMP), Bar=25μ. A′: High magnification of inset reveals co-label of alpha-synuclein in axons stained by TuJ1. Bar=10μ. Panels A-C: show alpha-synuclein (A) and TuJ1 (B) in the neural network of the outer longitudinal muscle wall and co-label (C). Bar=100μ. Panels A′-C′: High magnification of inset in B show co-label (C′ yellow) with alpha-synuclein (A′) and TuJ1 (B′) Bar=10μ.

FIG. 6 provides exemplary high resolution esophageal manometry (HREM) for one pill swallow, in accordance with some embodiments. FIG. 6 shows composite HRM tracings during one or more swallows. FIG. 6A shows EGJ obstruction. FIG. 6B shows pan-esophageal pressurization. FIG. 6C shows diffuse esophageal spasm. FIG. 6D shows fragmented peristalsis (large break). FIG. 6E shows ineffective esophageal peristalsis. FIG. 6F shows a normal HRM.

FIG. 7 provides illustrative characteristics of the anal sphincter, in particular the resting and squeeze pressures and the sphincter lengths, in accordance with some embodiments. Box plot graphs show resting and squeeze pressures in mmHg (left) and sphincter lengths in cm (right). The plots display the distribution of data as: minimum (bottom whisker), median (line in box), third quartile (upper part of box), and maximum (top whisker).

FIG. 8 provides illustrative pie charts indicating the prevalence of defecatory dyssynergia, in accordance with some embodiments. Top panel: Composite figure (pie charts) highlighting several HRAM characteristics of the cohort. A: Balloon expulsion test; B: Percent prevalence of certain anal sphincter measurements, such as low internal anal sphincter (IAS) and low external anal sphincter (EAS), predisposing to fecal incontinence; normal sphincter profiles for both IAS and EAS; and high IAS and EAS (anismus) predisposing to constipation. C: Percent prevalence of abnormal balloon sensation tests (in red) denoting impaired rectal sensation. D: Percent prevalence of absent recto-anal inhibitory reflex (in red), suggestive of impaired recto-anal coordination. Bottom panel: Pie chart highlighting the prevalence of defecatory dyssynergia types (I-IV) in the cohort studied by HRAM.

FIG. 9 provides illustrative gastric emptying times, in accordance with some embodiments.

FIG. 10 provides exemplary genes implicated in Multisystem Lewy body disease and parkinsonism. Euclidean distances from idiopathic Parkinson's disease were calculated based on 29 factors for the 22 genetic forms. Information analyzed included gene, mutation types causing primary disease, inheritance and name of primary disease, age at onset, clinical presentation, neuropathology, and peripheral autonomic involvement.

FIG. 11 shows Multisystem Lewy body disease and parkinsonism allelic variants for three of the genes with well-established associations with MLBD, i.e., SNCA, LRRK2, and GBA. There are allelic differences in phenotype as well as differences that cannot be attributed to allelic variation. Information analyzed included gene, allelic variant, mean age of onset, disease duration, cases with pathology, and presentation of MLBD.

FIG. 12 provides exemplary LRRK2 mutant rodent models and their phenotypes. In some embodiments, the LRRK2 mutant rodent model FVB Tg^{LRRK2(R1441G)6}, as disclosed by Bichler et al. in 2013, presents with GI dysfunction with changes in stool water content and dry weight.

FIG. 13 shows exemplary data resources for research on MLBD and other disorders with parkinsonism. Data sources are generally categorized as two types: 1) brain, tissue and clinical resources; and 2) genetic MLBD or parkinsonism resources. In some embodiments, tissues were collected from subjects with clinical and neuropathological diagnoses of MLBD and parkinsonism due to a variety of causes (such as multiple system atrophy) and additional diseases that show a-synuclein- and/or tau-related neuropathology (upper part of FIG. 13). Within these categories, samples from subjects with genetically causal forms of these disorders were also collected (lower part of FIG. 13).

FIG. 14 shows exemplary critical factors of intrinsic and extrinsic variability for iPS cell modeling.

FIG. 15 shows 21 clinical symptoms and eight categories derived from FIG. 10 and provides data on the distance metrics used in FIG. 1.

FIG. 16 shows the MLBD protein interaction networks, with more than 50 overlapping interactions.

### DETAILED DESCRIPTION

Complex, multi-symptom neurological diseases or conditions with a wide-range of etiologies, such as Parkinson's disease (PD), dementia, and Alzheimer's disease, are difficult to diagnose and distinguish from other similar diseases or conditions, as patients exhibit a broad range of symptoms that are associated with multiple genetic mutations and allelic variations among patients. The traditional method of diagnosing patients in such complex disease lacked a systematic way of distinguishing one disease from similar or related diseases with overlapping symptoms. Correlations between a genetic biomarker and a set of symptoms could be misleading in such scenarios, as correlations are not always indicative of causation, which is critical for accurate diagnosis and treatment.

Using Parkinson's disease and Parkinson-like (or parkinsonian) diseases as a case study, the present disclosure describes a novel approach for analyzing patient data and systematically redefining complex, multi-symptom neurological diseases or conditions into subclasses based on a combination of factors, such as gene, mutation types causing primary disease, inheritance and name of primary disease, age at onset, clinical presentation, neuropathology, and peripheral autonomic involvement (e.g., cardiac or gastrointestinal measurements) to delineate a common pathway or mechanism for different subclasses of disease/condition. Such systematic approach takes into account genetic and allelic variations and patient variation in clinical and pathological presentations of a complex disease or condition, allowing one to discover naturally occurring clusters within such systematic analysis of patient data based on prevalence of the measured factors in patients and linkage to a common biological pathway or underlying mechanism for each subclass. Such approach has the advantage of being more evidence-based and provides more objective and quantitative measures for more consistent and accurate diagnosis of a disease or condition, as different subclasses within a large class of complex diseases or conditions often require different and more targeted treatment.

In the case of Parkinson's disease and parkinsonian diseases, such method led to the identification of multisystem Lewy body disease (MLBD) and evidence that only three genes, i.e., LRRK2, SNCA, and GBA, show significant overlap in protein interactions that underlie Parkinson's disease, as other genes previously attributed to similar symptoms do not naturally cluster with SNCA, LRRK2, and GBA when one applied this multivariant approach to patient data analysis and clustering. Application of this novel approach of classifying or parsing out a complex set of diseases and conditions reveals distinct subclasses of parkinsonian diseases. This method of classifying and parsing out complex set of diseases or conditions in distinct subclasses based on the clustering of factors indicative of the underlying mechanism can be applied to other complex diseases or conditions with a wide-range of etiologies, such as dementia or Alzheimer's. This approach provides a new way of classifying or analyzing complex genetic diseases or conditions. Such approach and evidence based on the clustering of common protein networks or pathway provides reliable methods for diagnosis and targeting treatment.

Also described herein are methods of diagnosis or treatment of MLBD or Parkinson's disease based on at least measurements of non-motor or pre-motor symptoms, or symptoms associated with the peripheral autonomic system before symptoms present in central nervous system (CNS), such as GI dysfunction or cardiac abnormality. Such measurements of symptoms of the peripheral autonomic system provide a means for early diagnosis of a neurological condition before cellular damage or symptoms exhibit in the CNS. Measurements of early symptoms of the peripheral autonomic system, such as the enteric nervous system, also provide a means for targeting treatment, including preventative treatment, to slow or prevent progression of a neurological disease or condition. The enteric nervous system of the GI tract can also provide a model for developing and screening novel drugs or neuroprotective agents that are also therapeutic for the CNS.

In one aspect, presented herein is a method to prioritize as multisystem Lewy body disease (MLBD) those genetic forms of Parkinson's disease that point the way toward a mechanistic understanding of the majority of sporadic disease. Pathological diagnosis of genetic subtypes offers the prospect of distinguishing different mechanistic trajectories with a common mutational etiology, differing outcomes from varying allelic bases, and those disease-associated variants that can be used in gene-environment analysis. Delineating parkinsonian disorders into subclasses on the basis of molecular mechanisms with well-characterized outcome expectations is the basis for refining these forms of neurodegeneration as research substrate through the use of cell models derived from affected individuals while ensuring that clinically collected data can be used for therapeutic decisions and research without increasing the noise and confusion engendered by the collection of data against a range of historically defined criteria.

### Definitions

Unless defined otherwise, all technical and scientific terms used herein have the same meaning as is commonly understood by one of skill in the art to which this invention belongs.

As used herein, “multisystem Lewy body disease” (MLBD) means a clinical and neuropathological entity that presents clinically with signs and symptoms consistent with Parkinson's disease (PD) including both motor and nonmotor symptoms. Neuropathologically there is Lewy body disease with alpha-synuclein-positive Lewy bodies and Lewy neurites in the brain (typically following Braak staging), spinal cord and peripheral autonomic nervous system.

As used herein, PD classic definition means clinical pathologic complex that presents clinically with bradykinesia, resting tremor and rigidity. Neuropathologically there are alpha-synuclein-positive Lewy bodies, Lewy neurites and neuronal cell loss in the substantia nigra.

As used herein, “parkinsonism” means a clinical complex that presents with rigidity, resting tremor and bradykinesia, typically occurring with any condition that interferes with basal ganglia function. Parkinsonism can result from a variety of causes including neurodegenerative disease, toxins and structural lesions.

Neurological conditions may comprise neurodegenerative diseases and disorders in which cells of the brain and/or spinal cord are lost. The brain and spinal cord are composed of neurons that perform different functions such as controlling movements, processing sensory information, and making decisions. Cells of the brain and spinal cord are not readily regenerated en masse, so excessive damage can be devastating. Neurodegenerative diseases result from deterioration of neurons or their myelin sheath which over time will lead to dysfunction and disabilities. Neurodegenerative diseases are crudely divided into two groups according to phenotypic effects, although these are not mutually exclusive: conditions causing problems with movements, such as ataxia, and conditions affecting memory and related to dementia. Neurological disorders include, but are not limited to, ADHD, Alzheimer's disease (AD), amyotrophic lateral sclerosis (ALS; Lou Gehrig's disease), Bell's Palsy, Cerebral Palsy, chemotherapy-induced neuropathies (e.g., from vincristine, paclitaxel, bortezomib), chorea-acanthocytosis, Creutzfeldt-Jakob Disease (CJD), progressive supranuclear palsy, corticobasal degeneration, fronto-temporal dementia, dementia, diabetes-induced neuropathies, diffuse Lewy body disease, Epilepsy, Essential Tremor, Friedreich's ataxia, Guillain-Barre Syndrome, Hemifacial Spasm, Huntington's disease (HD), Movement Disorders, Multiple Sclerosis, Multisystem Atrophy (MSA), Nervous System Tumors, Neurofibromatosis, Neuropathy, ocular diseases (ocular neuritis), Parkinson's disease (PD), Periodic Limb Movement Disorder, primary lateral sclerosis, Seizure Disorders, Tourette's Syndrome or Traumatic Brain Injury.

### MLBD and Various Genetic Forms of Parkinson's Disease

Parkinson's disease is a neurodegenerative disease. Many of the signs and symptoms associated with Parkinson's disease can precede typical Parkinson's disease, in some cases by many years. Involvement of the dopaminergic substantia nigra, which underlies the primary motor features of the disease, occurs at a time when the disease is well advanced at a neuropathological level, an observation that may account for the difficulties in successfully testing new drugs for potential disease modifying properties only after Parkinson's disease is evident. As a result, there is increasing interest in identifying pre-motor or prodromal signs and symptoms of Parkinson's disease in order to identify the disorder in its earliest stages, well before motor symptoms are in evidence. In one embodiment, a low-cost, non-invasive screening method is provided for pre-motor or prodromal Parkinson's disease. The motor features of Parkinson's disease are characterized by muscle rigidity, tremor, gait and postural abnormalities, a slowing of physical movement (bradykinesia) and, in extreme cases, a loss of physical movement (akinesia). The primary symptoms are the results of decreased stimulation of the motor cortex and other areas of the brain by the basal ganglia, normally caused by the insufficient formation and action of dopamine, which is produced in the dopaminergic neurons of the brain. The motor features of Parkinson's disease are just one component of a much more wide-spread disorder that causes an abundance of non-motor signs and symptoms, including olfactory dysfunction, REM sleep behavioral disorder (RBD), constipation, depression, and cognitive deficits. Importantly, many of these signs and symptoms can precede the motor symptoms by years to a decade or more.

### Parkinson's-Like Diseases

There are several other conditions that have the features of Parkinson's disease and are interchangeably referred to as Parkinson's-like disease, secondary Parkinsonism, Parkinson's syndrome, or atypical Parkinson's. These are neurological syndromes that can be characterized by tremor, hypokinesia, rigidity, and postural instability. The underlying causes of Parkinson's-like disease are numerous, and diagnosis can be complex. A wide-range of etiologies can lead to a similar set of symptoms, including some toxins, a few metabolic diseases, and a handful of non-Parkinson's Disease neurological conditions. A common cause is as a side effect of medications, mainly neuroleptic antipsychotics especially the phenothiazines (such as perphenazine and chlorpromazine), thioxanthenes (such as flupenthixol and zuclopenthixol) and butyrophenones (such as haloperidol (Haldol)), piperazines (such as ziprasidone), and rarely, antidepressants. Other causes include but are not limted to olivopontocerebellar degeneration, progressive supranuclear palsy, corticobasal degeneration, temporo-frontal dementia; drug induced like antipsychotics, prochlorperazine, metoclopromide; poisoning with carbon monoxide; head trauma; and Huntington's disease Parkinsonism. In some cases alpha-synucleinopathies can result in Parkinson's-like disease, secondary Parkinsonism, Parkinson's syndrome, or atypical Parkinson's. In a related embodiment the methods described herein are used to diagnose Parkinson's-like disease, secondary Parkinsonism, Parkinson's syndrome, atypical Parkinson's, or a alpha syncleinopathy.

The methods disclosed herein may comprise screening a subject to determine if the subject is suffering from or prone to a neurological disorder such as Parkinson's disease. The screening methods comprise behavioral, biophysical, biochemical, and imaging assays and observations as well as questionnaires to determine if the subject is at risk for or is suffering from the early stages of a neurological disorder (e.g., Parkinson's disease). Biophysical and behavioral observations, such as physical examination of a subject for outward symptoms of disease can be evaluated independently, or combined with questionnaires and biochemical/imaging assays. Each individual assay can also be utilized independently or combined with biophysical evaluations or other tests that are known in the art and associated with a particular neurological disorder/disease. Examples of biochemical assays include genetic screens for mutations and/or polymorphisms (e.g., SNPs analysis, short tandem repeat analysis), biomarker-based assays, protein expression assays, immunohistochemistry assays or any combinations thereof. Material for biochemical assays can be sampled from all bodily fluids and tissues. Commonly employed bodily fluids include but are not limited to blood, serum, plasma, saliva, urine, gastric and digestive fluid, tears, stool, semen, vaginal fluid, interstitial fluids derived from tumorous tissue, and cerebrospinal fluid. Methods of obtaining samples of bodily tissue and fluids include but are not limited to biopsy, cheek swabbing, nose swabbing, rectal swabbing, skin fat extraction or other collection strategies for obtaining a biological or chemical substance.

Screening the subject may include imaging and scanning with the use of, but not limited to Positron Emission Tomography (PET) scans, Magnetic Resonance Imaging (MRI) scans, and Single-Photon Emission Computerized Tomography (SPECT) scans. Cardiovascular abnormalities related to Parkinson's disease in a subset of patients can be identified by heart rate spectral analysis.

The methods may comprise screening the subject for early stage, development, or late-stage Parkinson's disease by screening for primary and secondary symptoms, as described herein immediately following. The subject may be screened for biochemical indications of disease e.g., genetic mutations and/or abnormal protein expression levels of genes and proteins, respectively, associated with a disorder, in some cases prior to any onset of symptoms such as changes in motor behavior.

### Motor (Primary) Symptoms:

There are various factors known in the art which are used to screen and diagnose a subject for various neurological disorders. For example, in one embodiment, a subject is examined to determine if the subject is suffering from Parkinson's disease by assessing presence of primary symptoms which include but are not limited to: bradykinesia, tremors, rigidity, impaired balance, or a change in gait.

Bradykinesia is slowness in voluntary movement. It produces difficulty initiating movement as well as difficulty completing movement once it is in progress. The delayed transmission of signals from the brain to the skeletal muscles, due to diminished dopamine, produces bradykinesia.

Tremors in the hands, fingers, forearm, or foot tend to occur when the limb is at rest but not when performing tasks. Tremor may occur in the mouth and chin as well.

Rigidity, or stiff muscles, may produce muscle pain and an expressionless, mask-like face. Rigidity tends to increase during movement.

Poor and impaired balance is due to the impairment or loss of the reflexes that adjust posture in order to maintain balance. Falls are common in people with Parkinson's.

Parkinsonian gait is the distinctive unsteady walk associated with Parkinson's disease. There is a tendency to lean unnaturally backward or forward, and to develop a stooped, head-down, shoulders-drooped stance. Arm swing is diminished or absent and people with Parkinson's tend to take small shuffling steps (called festination). Someone with Parkinson's may have trouble starting to walk, appear to be falling forward as they walk, freeze in mid-stride, and have difficulty making a turn.

### Non-Motor (Secondary) Symptoms:

In some embodiments, progressive loss of voluntary and involuntary muscle control produces a number of secondary symptoms associated with Parkinson's disease. In some embodiments these symptoms are indicative of onset of primary symptoms. In other embodiments secondary symptoms can be in the absence of diagnosable motor symptoms, or present with primary symptoms. These symptoms can develop well before, shortly before, during, or after the onset and development of primary symptoms. In some cases, a subject can experience and display these symptoms about 50, 40, 30, 20, 15, 10, 5, 2 years, 1 year or 6 months before or 6 months, 1, 2, 5, 10, 15, 20, 30, 40, or more years after onset and display of primary symptoms. Some patients develop these secondary symptoms well before, years before the patients develop primary symptoms characteristic with a disorder.

Some secondary symptoms of Parkinson's disease include but are not limited to the following: Constipation occurring in a subject's 20's, 30's 40's or 50's; difficulty swallowing (dysphagia), saliva and food that collects in the mouth or back of the throat may cause choking, coughing, or drooling; excessive salivation (hypersalivation), excessive sweating (hyperhidrosis), loss of bladder and/or bowel control (incontinence); loss of sense of smell, olfactory dysfunction (anosmia); rapid eye movement (REM) sleep behavior disorder and other sleep disorders; changes in the cardiac sympathetic denervation, changes in the sympathetic innervation of the heart; loss of intellectual capacity (dementia), psychosocial: anxiety, depression, isolation; scaling, dry skin on the face and scalp (seborrhea); slow response to questions (bradyphrenia); small, cramped handwriting (micrographia); soft, whispery voice (hypophonia), and fatigue.

Therefore, in certain embodiments, diagnosis is based on symptoms and ruling out other disorders that produce similar symptoms. However to make a diagnosis of typical Parkinson's disease, a subject must have two or more of the diagnosable motor symptoms, one of which is a resting tremor or bradykinesia. In many cases, this diagnosis is made after observing that symptoms have developed and become established over a period of time. Such diagnostic techniques described above are known in the art.

After a thorough neurological exam and medical history, the neurologist may order computerized tomography (CT scan) or magnetic resonance imaging (MRI scan) to meet the other criterion for a diagnosis of Parkinson's disease: ruling out disorders (e.g., brain tumor, stroke) that produce Parkinson's-like symptoms. Some examples follow: medications—antipsychotics (e.g., Haldol) and anti-emetics (e.g., Compazine); multiple strokes; hydrocephalus; progressive supranuclear palsy—degeneration of midbrain structures; Shy-Drager syndrome—atrophy of central and sympathetic nervous systems; Wilson's disease—copper excretion causes degeneration of the liver and basal ganglia; Blood and/or cerebrospinal fluid (CSF) analysis may be ordered to look for specific abnormalities associated with other disorders.

In some embodiments, diagnosis is based on secondary non-motor symptoms even when the subject show no or very few of the primary motor symptoms associated with the neurological disease.

Primary and secondary symptoms may be insufficient to indicate disease risk or onset, and/or therapeutic efficacy. Genetic, biochemical and other types of screens presented hereforth can be conducted to determine if the subject is at risk for developing a neurological disorder (e.g., Parkinson's disease or Alzheimer's disease).

### Clinical Syndromes and Clinical Pathological Syndromes

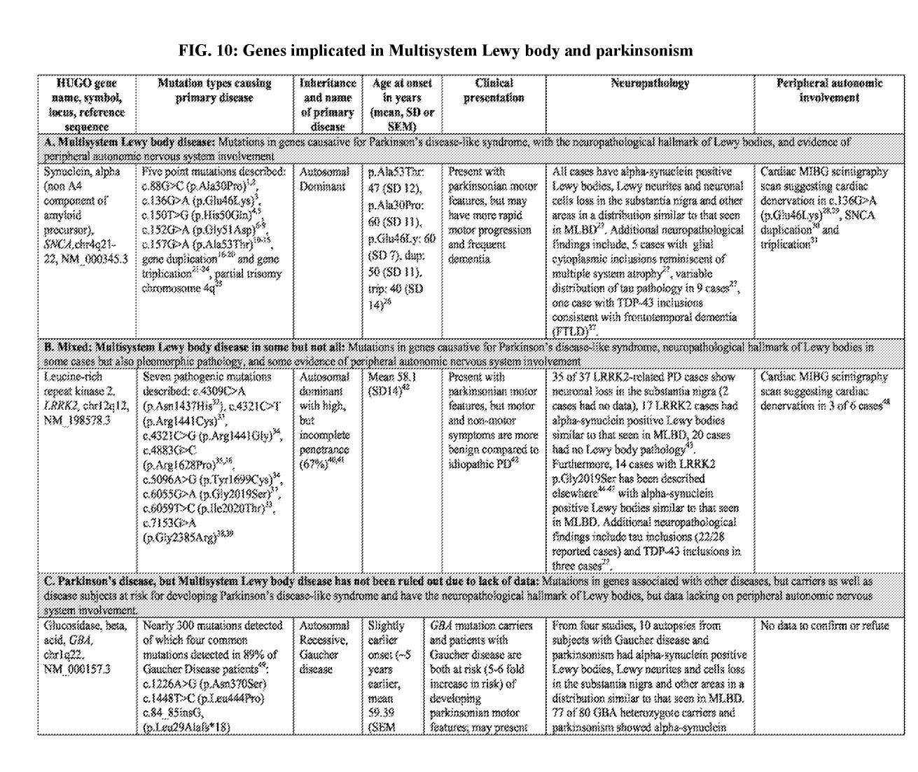

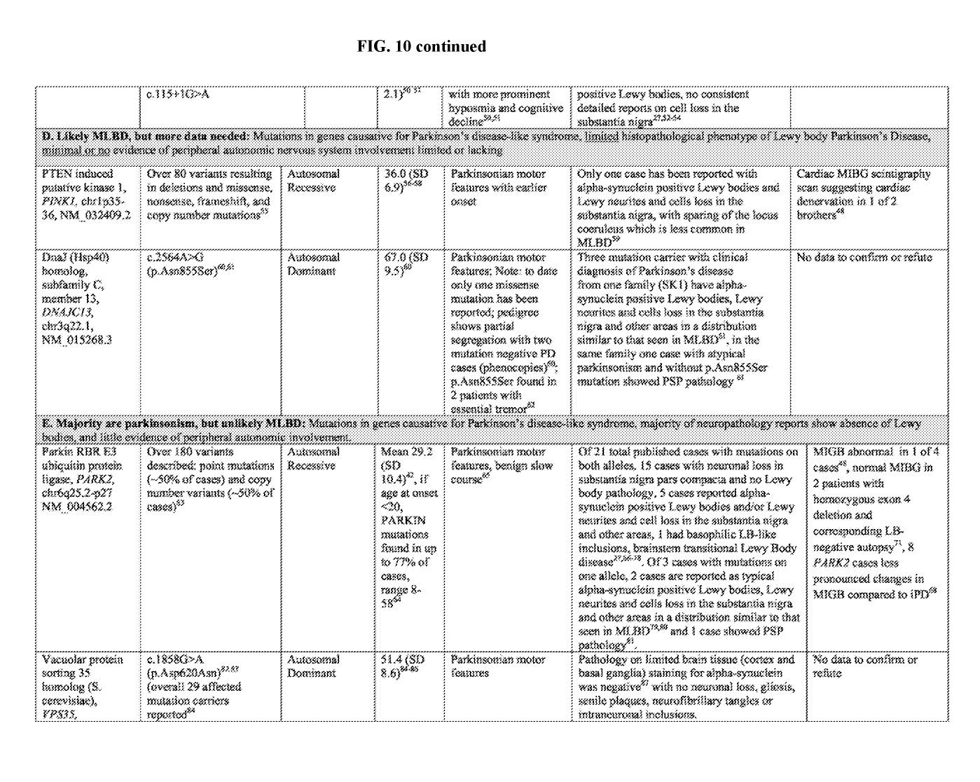

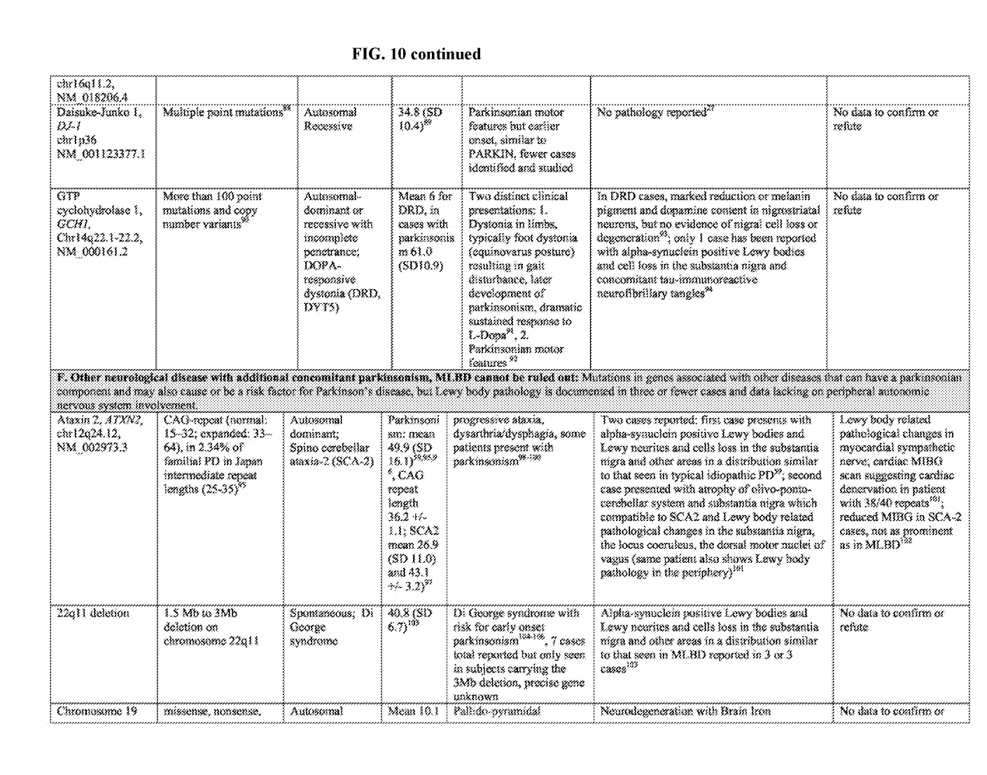

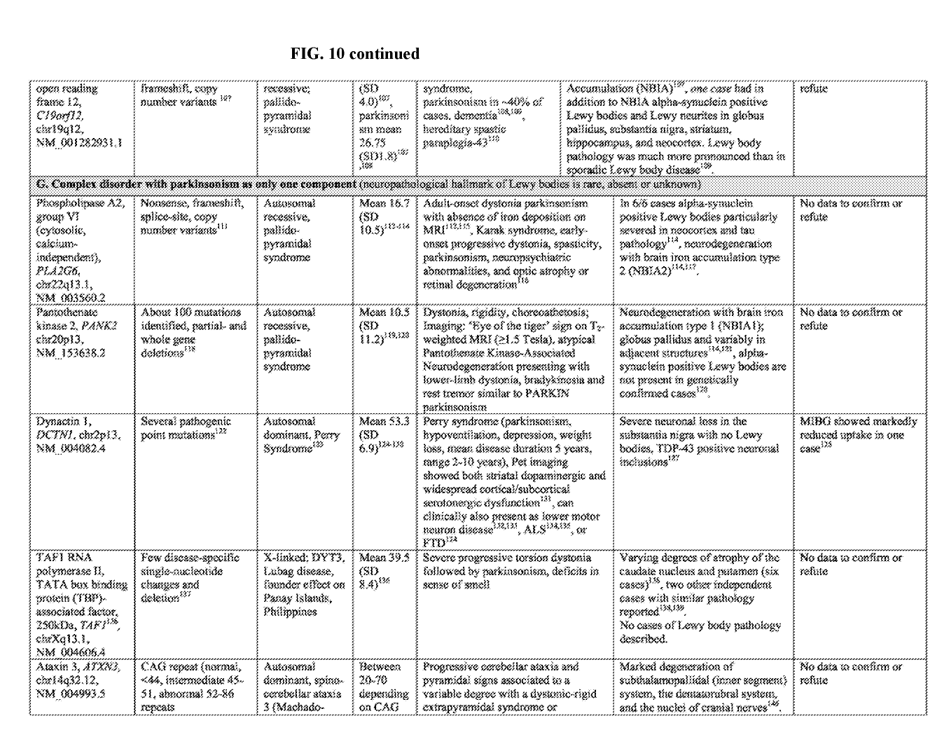

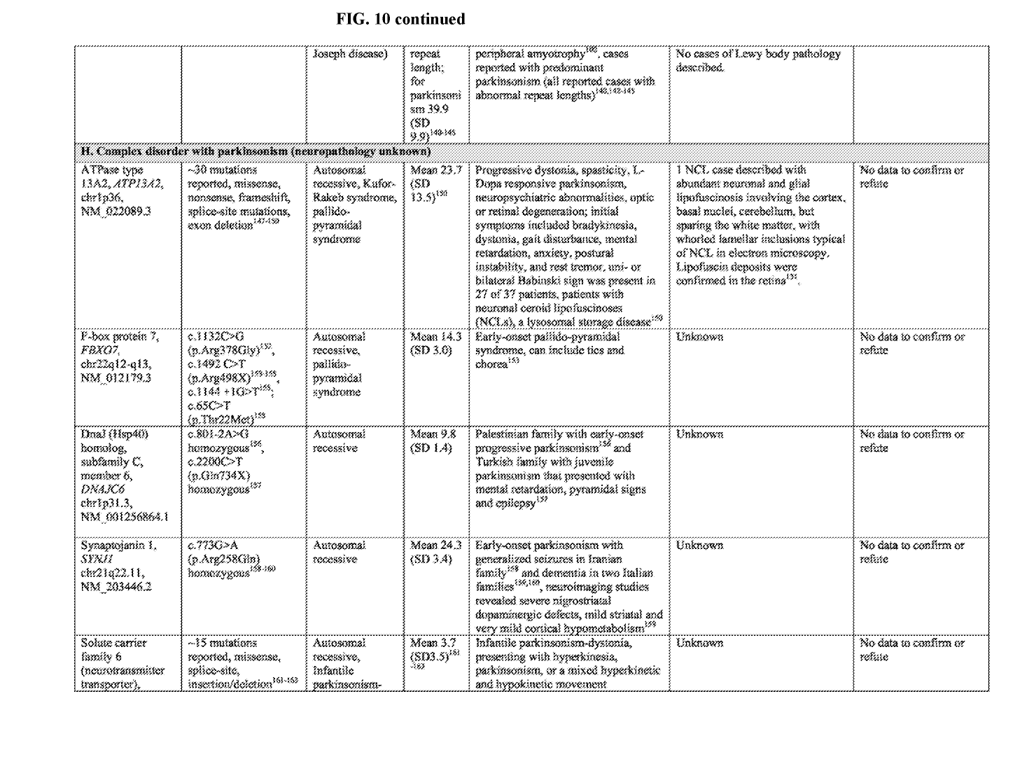

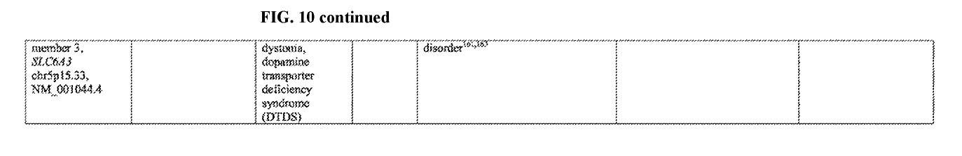

The five clinical and histopathological features of Parkinson's disease describe only a subset of what now appears to be a broader unitary disease process (6), while a set of related parkinsonian disorders that may have entirely different pathophysiological mechanisms are swept relatively unexamined into the Parkinson's disease classification (see TABLE 1 or FIG. 10). Because some genetic causes and some of the molecular entities responsible for this disease mechanism are now known, many have called for reclassification of parkinsonian nosology (7-11).

There is evidence that Parkinson's disease is part of a much more extensive process (6) that involves more than just the substantia nigra. Alpha-synuclein-positive Lewy neurites and Lewy bodies have repeatedly been reported in multiple areas of the brain and spinal cord and in the peripheral autonomic nervous system (6, 7, 12). Friedrich H. Lewy himself first identified the intracellular inclusions named after him not in the substantia nigra but the locus coeruleus, dorsomotor nucleus of the vagus and nucleus basalis of Meynert (8), and E. Herzog reported them in the peripheral autonomic nervous system as early as 1928 (9). Furthermore, it is now known that in the brain, Lewy pathology is typically first seen in the olfactory bulb and the dorsomo-tor nucleus of the vagus (10, 11, 13). Lewy pathology then progresses in a fairly typical pattern, from brain stem through a transitional phase to a diffuse disease. (11, 14) Braak has divided this ascending pathology into six stages (11), with the substantia nigra not affected until stage 3. Clinical penetrance of affected anatomical areas varies widely, and patients with Lewy pathology can present symptoms and signs ranging from constipation to dementia.

Two other classic Lewy body disorders, dementia with Lewy bodies (DLB) and pure autonomic failure (PAF), have been shown to have histopathological features virtually identical to those of Parkinson's disease and Parkinson's disease dementia (PDD) (15), suggesting that they are all parts of the same disease. The peripheral autonomic nervous system is also very important. a-synuclein-positive Lewy bodies and Lewy neurites have been identified postmortem in a wide variety of areas of the body, ranging from the myenteric plexus of the gut to the salivary gland, in patients diagnosed with Parkinson's disease (16). Over 40 imaging studies have shown sympathetic denervation of the heart in virtually all patients clinically diagnosed with Parkinson's disease, and one recent study showed Lewy neurites in the heart in 100% of the autopsy cases (17). Given the pathological distribution (brain, spinal cord and peripheral autonomic nervous) and natural history of its caudal-to-rostral development pathologically, or “multisystem Lewy body disease” (MLBD) in one embodiment of the invention.

The other key point is that the significance of the term ‘MLBD’ contrasts with what is denoted by ‘parkinsonism’, a clinical term referring to the syndrome of resting tremor, bradykinesia and rigidity. Thus ‘parkinsonism’ refers to a symptom complex, not a disease. Indeed, there are a huge number of causes of parkinsonism beyond neurodegenerative disease, ranging from toxins to pharmacological agents and even neoplastic lesions. This distinction is very important, particularly when categorizing patients on the basis of phenotype.

### Gene Names are Labels, not Surrogate Mechanisms

With associations having been demonstrated between neurodegenerative disease that have any parkinsonian features and over 35 reported genes and other risk factors (5, 18, 19), it is becoming harder to infer the mechanisms that connect genetic forms with sporadic Lewy body disease. In some embodiments, claiming of newly associated genes with the PARK label should cease. This should not create mass confusion because all of the genes, except PARK2, that have been associated with parkinsonism (alone or with other features) can be cited using their existing HUGO Gene Nomenclature Committee—approved or other approved gene names (such as SNCA, LRRK2, GBA and DJ-1). Because different alleles can lead to different pathology and symptoms, before any newly discovered gene variants can be confirmed as causative for MLBD, it would be important to define the clinical phenotype and the neuropathology, as well as evidence using replicated association, transmission or recurrent de novo mutation criteria recommended by the American College of Medical Genetics (20), before codifying it as a gene with parkinsonism-causative mutations.

In some embodiments, TABLE 1 (see also FIG. 10) provides a structure exemplifying how the claimed invention could work. Each of the gene variants is categorized as to whether it causes an MLBD-like disorder or simply parkinsonism (either as a primary manifestation or as part of a more complex disease). If the disorder is a Lewy body disease, it is important to include any available data on peripheral autonomic involvement (such as cardiac imaging as shown in TABLE 1 and FIG. 10).

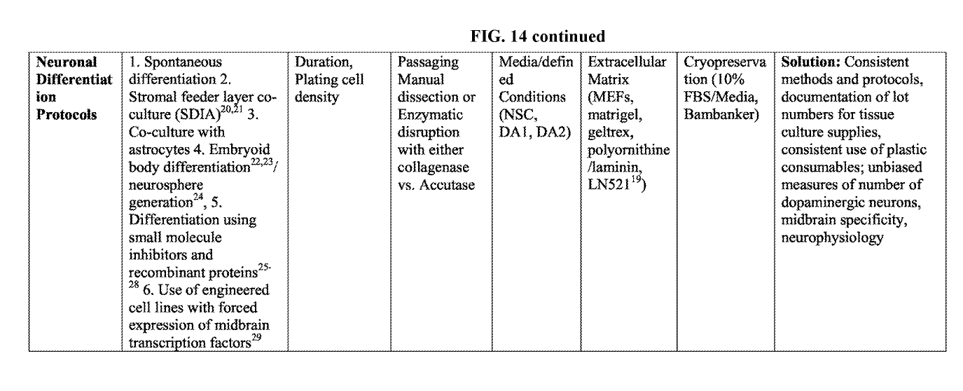

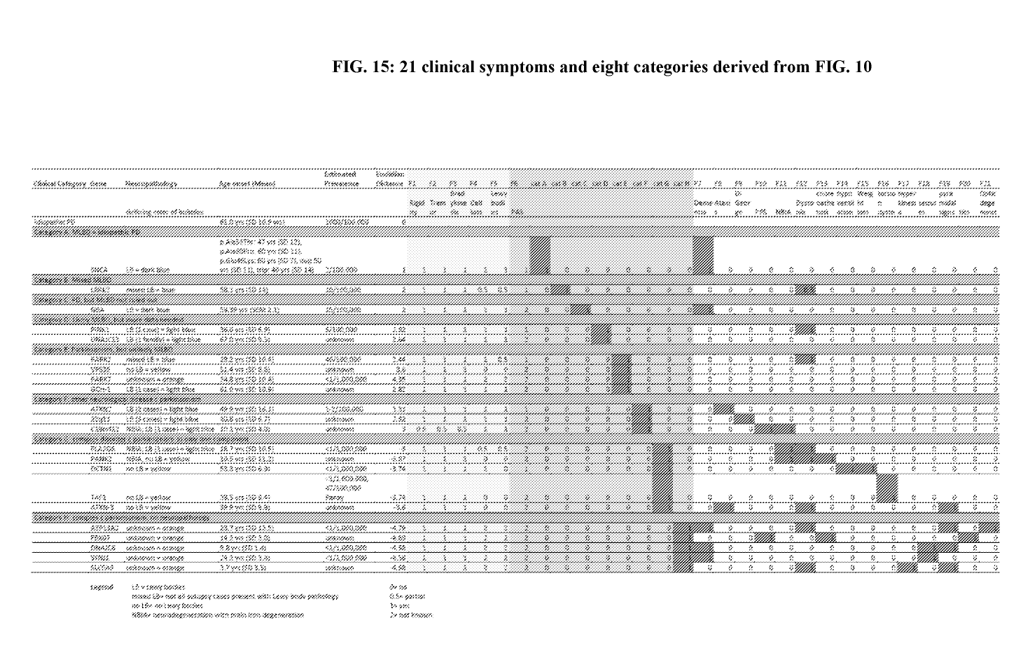

TABLE 1 (see also FIG. 10) below provides exemplary genes implicated in Multisystem Lewy body disease and parkinsonism. Euclidean distances from idiopathic Parkinson's disease were calculated based on 29 factors for the 22 genetic forms (see TABLE 6 for data on the distance metrics; see also FIG. 15). Information analyzed included gene, mutation types causing primary disease, inheritance and name of primary disease, age at onset, clinical presentation, neuropathology, and peripheral autonomic involvement

TABLE 1
Genes implicated in Multisystem Lewy body disease and parkinsonism

| | | | | | | |
|---|---|---|---|---|---|---|
| | | Inheritance of | | | | |
| | | primary | | | | |
| HUGO gene name, | Mutation types | disease, name | Age at onset in | | | Peripheral |
| symbol, locus, | causing primary | of primary | years (mean, SD or | Clinical | | autonomic |
| reference sequence | disease | disease | SEM) | presentation | Neuropathology | involvement |

A. Multisystem Lewy body disease: Mutations in genes causative for Parkinson's disease-like syndrome, with the neuropalhological hallmark of
Lewy bodies, and evidence of peripheral autonomic nervous system involvement

| | | | | | | |
|---|---|---|---|---|---|---|
| Synuclein, alpha | Five point mutations | Autosomal | p.Ala53Thr: 47 yrs | Present with | All cases have alpha-synuclein | Cardiac MIBG |
| (non A4 component | described: | Dominant | (SD 12), | parkinsonian | positive Lewy bodies, Lewy | scintigraphy scan |
| of amyloid | c.88G > C | | pAla30Pro: 60 yrs | motor features, | neurites and neuronal cells loss | suggesting cardiac |
| precursor), | (p.Ala30Pro)^{1,2}, | | (SD 11), | but may have | in the substantia nigra and | denervation in |
| SNCA, chr4q21-22, | c.136G > A | | p.Glu46Lys: 60 yrs | more rapid | other areas in a distribution | c.136G > A |
| NM_000345.3 | (p.Glu46Lys)³, | | (SD 7), dup 50 yrs | motor | similar to that seen in MLBD²¹. | (p.Glu46Lys)^{22,23} |
| | c. 150T > G | | (SD 11), trip: 40 yrs | progression and | Additional neuropathological | SNCA duplication²⁴ |
| | (p.His50Gln)^{4,5}, | | (SD 14)²⁰ | frequent | findings include, 5 cases with | and triplication²⁵ |
| | c. 152G > A | | | dementia | glial cytoplasmic inclusions | |
| | (p.Gly51 Asp)⁶, | | | | reminiscent of multiple system | |
| | c. 157G > A | | | | atrophy²¹, variable distribution | |
| | (p.Ala53Thr)⁷⁻¹² | | | | of tau pathology in 9 cases²¹, | |
| | gene duplication^{13,14} | | | | one case with TDP-43 | |
| | and gene | | | | inclusions consistent with | |
| | triplication¹⁵⁻¹⁸ | | | | frontotemporal dementia | |
| | partial trisomy | | | | (FTLD)²¹. | |
| | chromosome 4q¹⁹ | | | | | |

B. Mixed: Multisystem Lewy body disease in some but not all: Mutations in genes causative for Parkinson's disease-like syndrome,
neuropathological hallmark of Lewy bodies in some cases but also pleomorphic pathology, and some evidence of peripheral autonomic nervous
system involvement

| | | | | | | |
|---|---|---|---|---|---|---|
| Leucine-rich repeat | Seven pathogenic | Autosomal | Mean 58.1 yrs | Present with | 35 of 37 LRRK2-related PD | Cardiac MIBG |
| kinase 2, LRRK2, | mutations described: | dominant with | (SD14)³⁶ | parkinsonian | cases show neuronal loss in the | scintigraphy scan |
| chr12q12, | c.4309C > A | high, but | | motor features, | substantia nigra (2 cases had no | suggesting cardiac |
| NM_198578.3 | (p.Asn1437His²⁶), | incomplete | | but motor and | data), 17 LRRK2 cases had | denervation in 3 of 6 |
| | c.4321C > T | penetrance | | non-motor | alpha-synuclein positive Lewy | cases⁴² |
| | p.Arg1441Cys)²⁷, | (67%)^{34,35} | | symptoms are | bodies similar to that seen in | |
| | c.4321C > G | | | more benign | MLBD, 20 cases had no Lewy | |
| | (p.Arg1441Gly)²⁸, | | | compared to | body pathology³⁷. Furthermore, | |
| | c.4883G > C | | | idiopathic PD³⁶ | 14 cases with LRRK2 | |
| | (p.Arg1628Pro)^{29,30}, | | | | p.Gly2019Ser has been | |
| | c.5096A > G | | | | described elsewhere³⁸⁻⁴¹ with | |
| | (p.Tyr1699Cys)²⁸, | | | | alpha-synuclein positive Lewy | |
| | c.6055G > A | | | | bodies similar to that seen in | |
| | (p.Gly2019Ser)³¹, | | | | MLBD. Additional | |
| | c.6059T > C | | | | neuropathological findings | |
| | (p.Ile2020Thr)²⁷, | | | | include tau inclusions (22/28 | |
| | c.7153G > A | | | | reported cases) and TDP-43 | |
| | (p.Gly2385Arg)^{32,33} | | | | inclusions in three cases²¹. | |

C. Parkinson's disease, but Multisystem Lewy body disease has not been ruled out due to lack of data: Mutations in genes associated with other
diseases, but carriers as well as disease subjects at risk for developing Parkinson's disease-like syndrome and have the neuropathological hallmark of
Lewy bodies, but data lacking on peripheral autonomic nervous system involvement.

| | | | | | | |
|---|---|---|---|---|---|---|
| Glucosidase, beta, | Nearly 300 | Autosomal | Slightly earlier onset | GBA mutation | From four studies, 10 autopsies | No data to confirm |
| acid, GBA, chr1q22, | mutations detected | Recessive, | (~5 years earlier, | carriers and | from subjects with Gaucher | or refute |
| NM_000157.3 | of which four | Gaucher | mean 59.39 yrs | patients with | disease and parkinsonism had | |
| | common mutations | disease | (SEM 2.1)^{44 45} | Gaucher | alpha-synuclein positive Lewy | |
| | detected in 89% of | | | disease are both | bodies, Lewy neurites and cells | |
| | Gaucher Disease | | | at risk (5-6 fold | loss in the substantia nigra and | |
| | patients⁴³: | | | increase in risk) | other areas in a distribution | |
| | c.1226A > G | | | of developing | similar to that seen in MLBD. | |
| | (p.Asn370Ser) | | | parkinsonian | 77 of 80 GBA heterozygote | |
| | c.1448T > C | | | motor features; | carriers and parkinsonism | |
| | (p.Leu444Pro) | | | may present | showed alpha-synuclein | |
| | c.84_85insG, | | | with more | positive Lewy bodies, no | |
| | (p.Leu29Alafs*18) | | | prominent | consistent detailed reports on | |
| | c.115 + 1G > A | | | hyposmia and | cell loss in the substantia nigra²¹ | |
| | | | | cognitive | | |
| | | | | decline^{44,45}_ENREF_37 | | |

D. Likely MLBD, but more data needed: Mutations in genes causative for Parkinson's disease-like syndrome, limited histopathological phenotype
of Lewy body Parkinson's Disease, minimal or no evidence of peripheral autonomic nervous system involvement limited or lacking

| | | | | | | |
|---|---|---|---|---|---|---|
| PTEN induced | Over 80 variants | Autosomal | 36.0 yrs (SD 6.9)⁴⁷⁻⁴⁹ | Parkinsonian | Only one case has been | Cardiac MIBG |
| putative kinase 1, | resulting in deletions | Recessive | | motor features | reported with alpha-synuclein | scintigraphy scan |
| PINK1, chr1p35-36, | and missense, | | | with earlier | positive Lewy bodies and | suggesting cardiac |
| NM_032409.2 | nonsense, | | | onset | Lewy neurites and cells loss in | denervation in 1 of 2 |
| | frameshift, and copy | | | | the substantia nigra, with | brothers⁴² |
| | number mutations⁴⁶ | | | | sparing of the locus coeruleus | |
| | | | | | which is less common in | |
| | | | | | MLBD⁵⁰ | |
| DnaJ (Hsp40) | c.2564A > G | Autosomal | 67.0 yrs (SD 9.5)⁵¹ | Parkinsonian | Three mutation carrier with | No data to confirm |
| homolog, subfamily | (p.Asn855Ser)^{51,52} | Dominant | | motor features; | clinical diagnosis of | or refute |
| C, member 13, | | | | Note: to date | Parkinson's disease | |
| DNAJC13, | | | | only one | from one family (SK1) have | |
| chr3q22.1, | | | | missense | alpha-synuclein positive Lewy | |
| NM_015268.3 | | | | mutation has | bodies, Lewy neurites and cells | |
| | | | | been reported; | loss in the substantia nigra and | |
| | | | | pedigree shows | other areas in a distribution | |
| | | | | partial | similar to that seen in MLBD⁵², | |
| | | | | segregation | in the same family one case | |
| | | | | with two | with atypical parkinsonism and | |
| | | | | mutation | without p.Asn855Ser mutation | |
| | | | | negative PD | showed PSP pathology⁵² | |
| | | | | cases | | |
| | | | | (phenocopies)⁵¹; | | |
| | | | | p.Asn855Ser | | |
| | | | | found in 2 | | |
| | | | | patients with | | |
| | | | | essential | | |
| | | | | tremor⁵³ | | |

E. Majority are parkinsonism, but unlikely MLBD: Mutations in genes causative for Parkinson's disease-like syndrome, majority of
neuropathology reports show absence of Lewy bodies, and little evidence of peripheral autonomic involvement.

| | | | | | | |
|---|---|---|---|---|---|---|
| Parkin RBR E3 | Over 180 variants | Autosomal | Mean 29.2 yrs (SD | Parkinsonian | Of 21 total published cases | MIGB abnormal in |
| ubiquitin protein | described: point | Recessive | 10.4)³⁶, if age at | motor features, | with mutations on both alleles, | 1 of 4 cases⁴², |
| ligase, PARK2, | mutations (~50% of | | onset < 20 years, | benign slow | 15 cases with neuronal loss in | normal MIBG in 2 |
| chr6q25.2-p27 | cases) and copy | | PARKIN mutations | course⁵⁶ | substantia nigra pars compacta | patients with |
| NM_004562.2 | number variants | | found in up to 77% | | and no Lewy body pathology, 5 | homozygous exon 4 |
| | (~50% of cases)⁵⁴ | | of cases, range 8-58 | | cases reported alpha-synuclein | deletion and |
| | | | years⁵⁵ | | positive Lewy bodies and/or | corresponding LB- |
| | | | | | Lewy neurites and cell loss in | negative autopsy⁶², 8 |
| | | | | | the substantia nigra and other | PARK2 cases less |
| | | | | | areas, 1 had basophilic LB-like | pronounced changes |
| | | | | | inclusions, brainstem | in MIGB compared |
| | | | | | transitional Lewy Body | to iPD⁵⁹ |
| | | | | | disease^{21,57,69}. Of 3 cases with | |
| | | | | | mutations on one allele, 2 cases | |
| | | | | | are reported as typical alpha- | |
| | | | | | synuclein positive Lewy | |
| | | | | | bodies, Lewy neurites and cells | |
| | | | | | loss in the substantia nigra and | |
| | | | | | other areas in a distribution | |
| | | | | | similar to that seen in | |
| | | | | | MLBD^{70,71} and 1 case showed | |
| | | | | | PSP pathology⁷². | |
| Vacuolar protein | c. 1858G > A | Autosomal | 51.4 yrs (SD 8.6)⁷⁵⁻⁷⁷ | Parkinsonian | Pathology on limited brain | No data to confirm |
| sorting 35 homolog | (p.Asp620Asn)^{73,74} | Dominant | | motor features | tissue (cortex and basal | or refute |
| (S. cerevisiae), | (overall 29 affected | | | | ganglia) staining for alpha- | |
| VPS35, chr16q11.2, | mutation carriers | | | | synuclein was negative⁷⁸ with | |
| NM_018206.4 | reported⁷⁵ | | | | no neuronal loss, gliosis, senile | |
| | | | | | plaques, neurofibrillary tangles | |
| | | | | | or intraneuronal inclusions. | |
| Daisuke-Junko 1, | Multiple point | Autosomal | 34.8 yrs (SD 10.4)⁸⁰ | Parkinsonian | No pathology reported²¹ | No data to confirm |
| DJ-1 | mutations⁷⁹ | Recessive | | motor features | | or refute |
| chr1p36 | | | | but earlier | | |
| NM_001123377.1 | | | | onset, similar to | | |
| | | | | PARKIN, fewer | | |
| | | | | cases identified | | |
| | | | | and studied | | |
| GTP cyclohydrolase | More than 100 point | Autosomal- | Mean 6 yrs for DRD, | Two distinct | In DRD cases, marked | No data to confirm 2 |
| 1, GCH1, | mutations and copy | dominant or | in cases with | clinical | reduction or melanin pigment | or refute |
| Chr14q22.1-22.2, | number variants⁸¹ | recessive with | parkinsonism 61.0 | presentations: | and dopamine content in | |
| NM_000161.2 | | incomplete | yrs (SD10.9) | 1. Dystonia in | nigrostriatal neurons, but no | |
| | | penetrance; | | limbs, typically | evidence of nigral cell loss or | |
| | | DOPA- | | foot dystonia | degeneration⁸⁴; only 1 case has | |
| | | responsive | | (equinovarus | been reported with alpha- | |
| | | dystonia | | posture) | synuclein positive Lewy bodies | |
| | | (DRD, DYT5) | | resulting in gait | and cell loss in the substantia | |
| | | | | disturbance, | nigra and concomitant tau- | |
| | | | | later | immunoreactive neurofibrillary | |
| | | | | development of | tangles⁸⁵ | |
| | | | | parkinsonism, | | |
| | | | | dramatic | | |
| | | | | sustained | | |
| | | | | response to L- | | |
| | | | | Dopa⁸², 2. | | |
| | | | | Parkinsonian | | |
| | | | | motor features⁸³ | | |

F. Other neurological disease with additional concomitant parkinsonism, MLBD cannot be ruled out: Mutations in genes associated with other
diseases that can have a parkinsonian component and may also cause or be a risk factor for Parkinson's disease, but Lewy body pathology is
documented in three or fewer cases and data lacking on peripheral autonomic nervous system involvement.

| | | | | | | |
|---|---|---|---|---|---|---|
| Ataxin 2, ATXN2, | CAG-repeat | Autosomal | Parkinsonism: mean | progressive | Two cases reported: first case | Lewy body related |
| chr12q24.12, | (normal: 15-32; | dominant; | 49.9 yrs (SD | ataxia, | presents with alpha-synuclein | pathological changes |
| NM_002973.3 | expanded: 33-64), | Spino | 16.1)^{50,86,87}, CAG | dysarthria/dysp | positive Lewy bodies and | in myocardial |
| | in 2.34% of familial | cerebellar | length 36.2 +/− 1.1 | hagia, some | Lewy neurites and cells loss in | sympathetic nerve; |
| | PD in Japan | ataxia-2 (SCA- | repeat; SCA2 mean | patients present | the substantia nigra and other | cardiac MIBG scan |
| | intermediate repeat | 2) | 26.9 yrs (SD 11.0) | with | areas in a distribution similar to | suggesting cardiac |
| | lengths (25-35)⁸⁶ | | and 43.1 +/− 3.2 | parkinsonism⁸⁹⁻⁹¹ | that seen in typical idiopathic | denervation in |
| | | | repeats)⁸⁸ | | PD⁵⁰; second case presented | patient with 38/40 |
| | | | | | with atrophy of olivo-ponto- | repeats⁹²; reduced |
| | | | | | cerebellar system and | MIBG in SCA-2 |
| | | | | | substantia nigra which | cases, not as |
| | | | | | compatible to SCA2 and Lewy | prominent as in |
| | | | | | body related pathological | MLBD⁹³ |
| | | | | | changes in the substantia nigra, | |
| | | | | | the locus coeruleus, the dorsal | |
| | | | | | motor nuclei of vagus (same | |
| | | | | | patient also shows Lewy body | |
| | | | | | pathology in the periphery)⁹² | |
| 22q11 deletion | 1.5 Mb to 3 Mb | Spontaneous; | 40.8 yrs (SD 6.7)⁹⁴ | Di George | Alpha-synuclein positive Lewy | No data to confirm |
| | deletion on | Di George | | syndrome with | bodies and Lewy neurites and | or refute |
| | chromosome 22q11 | syndrome | | risk for early | cells loss in the substantia nigra | |
| | | | | onset | and other areas in a distribution | |
| | | | | parkinsonism⁹⁵⁻⁹⁷, | similar to that seen in MLBD | |
| | | | | 7 cases total | reported in 3 or 3 cases⁹⁴ | |
| | | | | reported but | | |
| | | | | only seen in | | |
| | | | | subjects | | |
| | | | | carrying the | | |
| | | | | 3 Mb deletion, | | |
| | | | | precise gene | | |
| | | | | unknown | | |
| Chromosome 19 | missense, nonsense, | Autosomal | Mean 10.1 years | Pallido- | Neurodegeneration with Brain | No data to confirm |
| open reading frame | frameshift, copy | recessive; | (SD 4.0)⁹⁸, | pyramidal | Iron Accumulation (NBIA)⁹⁸, | or refute |
| 12, C19orf12, | number variants⁹⁸ | pallido- | parkinsonism mean | syndrome, | one case had in addition to | |
| chr19q12, | | pyramidal | 26.75 yrs | parkinsonism in | NBIA alpha-synuclein positive | |
| NM_001282931.1 | | syndrome | (SD1.8)^{98,99} | ~40% of cases, | Lewy bodies and Lewy | |
| | | | | dementia^{99,100}, | neurites in globus pallidus, | |
| | | | | hereditary | substantia nigra, striatum, | |
| | | | | spastic | hippocampus, and neocortex. | |
| | | | | parapiegia-43¹⁰¹ | Lewy body pathology was | |
| | | | | | much more pronounced than in | |
| | | | | | sporadic Lewy body disease¹⁰⁰. | |

G. Complex disorder with parkinsonism as only one component (neuropathological hallmark of Lewy bodies is rare, absent or unknown)

| | | | | | | |
|---|---|---|---|---|---|---|
| Phospholipase A2, | Nonsense, | Autosomal | Mean 16.7 yrs (SD | Adult-onset | In 6/6 cases alpha-synuclein | No data to confirm |
| group VI (cytosolic, | frameshift, splice- | recessive, | 10.5)¹⁰³⁻¹⁰⁵ | dystonia | positive Lewy bodies | or refute |
| calcium- | site, copy number | pallido- | | parkinsonism | particularly severed in | |
| independent), | variants¹⁰² | pyramidal | | with absence of | neocortex and tau pathology¹⁰⁵, | |
| PLA2G6, | | syndrome | | iron deposition | neurodegeneration with brain | |
| chr22q13.1, | | | | on MRI^{103,106}, | iron accumulation type 2 | |
| NM_003560.2 | | | | Karak | (NBIA2)^{105,108}. | |
| | | | | syndrome, | | |
| | | | | early-onset | | |
| | | | | progressive | | |
| | | | | dystonia, | | |
| | | | | spasticity, | | |
| | | | | parkinsonism, | | |
| | | | | neuropsychiatric | | |
| | | | | abnormalities, | | |
| | | | | and optic | | |
| | | | | atrophy or | | |
| | | | | retinal | | |
| | | | | degeneration¹⁰⁷ | | |
| Pantothenate kinase | About 100 mutations | Autosomal | Mean 10.5 yrs (SD | Dystonia, | Neurodegeneration with brain | No data to confirm |
| 2, PANK2 | identified, partial- | recessive, | 11.2)^{110,111} | rigidity, | iron accumulation type 1 | or refute |
| chr20p13, | and whole gene | pallido- | | choreoathetosis; | (NBIA1); globus pallidus and | |
| NM_153638.2 | deletions¹⁰⁹ | pyramidal | | Imaging: ‘Eye | variably in adjacent | |
| | | syndrome | | of the tiger’ | structures^{105,112}, alpha- | |
| | | | | sign on T₂- | synuclein positive Lewy bodies | |
| | | | | weighted MPI | are not present in genetically | |
| | | | | (≥1.5 Tesla), | confirmed cases¹¹¹. | |
| | | | | atypical | | |
| | | | | Pantothenate | | |
| | | | | Kinase- | | |
| | | | | Associated | | |
| | | | | Neurodegeneration | | |
| | | | | presenting | | |
| | | | | with lower-limb | | |
| | | | | dystonia, | | |
| | | | | bradykinesia | | |
| | | | | and rest tremor | | |
| | | | | similar to | | |
| | | | | PARKIN | | |
| | | | | parkinsonism | | |
| Dynactin 1, DCTN1, | Several pathogenic | Autosomal | Mean 53.3 yrs (SD | Perry syndrome | Severe neuronal loss in the | MIBG showed |
| chr2p13, | point mutations¹¹³ | dominant, | 6.9)¹¹⁵⁻¹²¹ | (parkinsonism, | substantia nigra with no Lewy | markedly reduced |
| NM_004082.4 | | Perry | | hypoventilation, | bodies, TDP-43 positive | uptake in one case¹¹⁶ |
| | | Syndrome¹¹⁴ | | depression, | neuronal inclusions¹¹⁸ | |
| | | | | weight loss, | | |
| | | | | mean disease | | |
| | | | | duration 5 | | |
| | | | | years, range 2-10 | | |
| | | | | years), Pet | | |
| | | | | imaging | | |
| | | | | showed both | | |
| | | | | striatal | | |
| | | | | dopaminergic | | |
| | | | | and widespread | | |
| | | | | cortical/subcortical | | |
| | | | | serotonergic | | |
| | | | | dysfunction¹²², | | |
| | | | | can clinically | | |
| | | | | also present as | | |
| | | | | lower motor | | |
| | | | | neuron | | |
| | | | | disease^{123,124}, | | |
| | | | | ALS^{125,126}, or | | |
| | | | | FTD¹¹⁵ | | |
| TAF1 RNA | Few disease-specific | X-linked; | Mean 39.5 years | Severe | Varying degrees of atrophy of | No data to confirm |
| polymerase II, | single-nucleotide | DYT3, Lubag | (SD 8.4)¹²⁷ | progressive | the caudate nucleus and | or refute |
| TATA box binding | changes and | disease, | | torsion dystonia | putamen (six cases)¹²⁷, two | |
| protein (TBP)- | deletion¹²⁸ | founder effect | | followed by | other independent cases with | |
| associated factor, | | on Panay | | parkinsonism, | similar pathology | |
| 250 kDa, TAF1¹²⁷, | | Islands, | | deficits in sense | reported^{129,130}. | |
| chrXq13.1, | | Philippines | | of smell | No cases of Lewy body | |
| NM_004606.4 | | | | | pathology described. | |
| Ataxin 3, ATXN3, | CAG repeat | Autosomal | Between 20-70 yrs | Progressive | Marked degeneration of | No data to confirm |
| chr14q32.12, | (normal, > 44, | dominant, | depending on CAG | cerebellar | subthalamopallidal (inner | or refute |
| NM_004993.5 | intermediate 45-51, | spino- | repeat length; for | ataxia and | segment) system, the | |
| | abnormal 52-86 | cerebellar | parkinsonism 39.9 yrs | pyramidal signs | dentatorubral system, and the | |
| | repeats | ataxia 3 | (SD 9.9)¹³¹⁻¹³⁶_ENREF_127_ENREF_127 | associated to a | nuclei of cranial nerves¹³⁷. | |
| | | (Machado- | | variable degree | No cases of Lewy body | |
| | | Joseph | | with a dystonic- | pathology described. | |
| | | disease) | | rigid | | |
| | | | | extrapyramidal | | |
| | | | | syndrome or | | |
| | | | | peripheral | | |
| | | | | amyotrophy⁹¹, | | |
| | | | | cases reported | | |
| | | | | with | | |
| | | | | predominant | | |
| | | | | parkinsonism | | |
| | | | | (all reported | | |
| | | | | cases with | | |
| | | | | abnormal repeat | | |
| | | | | lengths)^{131,133-136} | | |

H. Complex disorder with parkinsonism (neuropathology unknown)

| | | | | | | |
|---|---|---|---|---|---|---|
| ATPase type 13A2, | ~30 mutations | Autosomal | Mean 23.7 yrs (SD | Progressive | 1 NCL case described with | No data to confirm |
| ATP13A2, | reported, missense, | recessive, | 13.5)¹⁴¹ | dystonia, | abundant neuronal and glial | or refute |
| chr1p36, | nonsense, | Kufor-Rakeb | | spasticity, L- | lipofuscinosis involving the | |
| NM_022089.3 | frameshift, splice- | syndrome, | | Dopa | cortex, basal nuclei, | |
| | site mutations, exon | pallido- | | responsive | cerebellum, but sparing the | |
| | deletion¹³⁸⁻¹⁴¹_ENREF_119_ENREF_119 | pyramidal | | parkinsonism, | white matter, with whorled | |
| | | syndrome | | neuropsychiatric | lamellar inclusions typical of | |
| | | | | abnormalities, | NCL in electron microscopy. | |
| | | | | optic or retinal | Lipofuscin deposits were | |
| | | | | degeneration; | confirmed in the retina¹⁴². | |
| | | | | initial | | |
| | | | | symptoms | | |
| | | | | included | | |
| | | | | bradykinesia, | | |
| | | | | dystonia, gait | | |
| | | | | disturbance, | | |
| | | | | mental | | |
| | | | | retardation, | | |
| | | | | anxiety, | | |
| | | | | postural | | |
| | | | | instability, and | | |
| | | | | rest tremor, uni- | | |
| | | | | or bilateral | | |
| | | | | Babinski sign | | |
| | | | | was present in | | |
| | | | | 27 of 37 | | |
| | | | | patients, | | |
| | | | | patients with | | |
| | | | | neuronal ceroid | | |
| | | | | lipofuscinoses | | |
| | | | | (NCLs), a | | |
| | | | | lysosomal | | |
| | | | | storage | | |
| | | | | disease¹⁴¹ | | |
| F-box protein 7, | c.1132 > G | Autosomal | Mean 14.3 yrs (SD | Early-onset | Unknown | No data to confirm |
| FBXO7, | (p.Arg378Gly)¹⁴³, | recessive, | 3.0) | pallido- | | or refute |
| chr22q12-q13, | c.1492 C > T | pallido- | | pyramidal | | |
| NM_012179.3 | (p.Arg498X)¹⁴⁴⁻¹⁴⁶, | pyramidal | | syndrome, can | | |
| | c.1144 + 1G > T¹⁴⁶; | syndrome | | include tics and | | |
| | c.65C > T | | | chorea¹⁴⁴ | | |
| | (p.Thr22Met)¹⁴⁶ | | | | | |
| DnaJ (Hsp40) | c.801-2A > G | Autosomal | Mean 9.8 yrs (SD | Palestinian | Unknown | No data to confirm |
| homolog, subfamily | homozygous¹⁴⁷, | recessive | 1.4) | family with | | or refute |
| C, member 6, | c.2200C > T | | | early-onset | | |
| DNAJC6 | (p.Gln734X) | | | progressive | | |
| chr1p31.3, | homozygous¹⁴⁸ | | | parkinsonism¹⁴⁷ | | |
| NM_001256864.1 | | | | and Turkish | | |
| | | | | family with | | |
| | | | | juvenile | | |
| | | | | parkinsonism | | |
| | | | | that presented | | |
| | | | | with mental | | |
| | | | | retardation, | | |
| | | | | pyramidal signs | | |
| | | | | and epilepsy¹⁴⁸ | | |
| Synaptojanin 1, | c.773G > A | Autosomal | Mean 24.3 yrs (SD | Early-onset | Unknown | No data to confirm |
| SYNJ1 | (p.Arg258Gln) | recessive | 3.4) | parkinsonism | | or refute |
| chr21q22.11, | homozygous¹⁴⁹⁻¹⁵¹ | | | with | | |
| NM_203446.2 | | | | generalized | | |
| | | | | seizures in | | |
| | | | | Iranian | | |
| | | | | family¹⁴⁹ and | | |
| | | | | dementia in two | | |
| | | | | Italian | | |
| | | | | families^{150,151}, | | |
| | | | | neuroimaging | | |
| | | | | studies revealed | | |
| | | | | severe | | |
| | | | | nigrostriatal | | |
| | | | | dopaminergic | | |
| | | | | defects, mild | | |
| | | | | striatal and very | | |
| | | | | mild cortical | | |
| | | | | hypometabolism¹⁵⁰ | | |
| Solute carrier family | ~15 mutations | Autosomal | Mean 3.7 yrs | Infantile | Unknown | No data to confirm |
| 6 (neurotransmitter | reported, missense, | recessive, | (SD3.5)¹⁵²⁻¹⁵⁴ | parkinsonism- | | or refute |
| transporter), member | splice-site, | Infantile | | dystonia, | | |
| 3, SLC6A3 | insertion/deletion¹⁵²⁻¹⁵⁴ | parkinsonism- | | presenting with | | |
| chr5p15.33, | | dystonia, | | hyperkinesia, | | |
| NM_001044.4 | | dopamine | | parkinsonism, | | |
| | | transporter | | or a mixed | | |
| | | deficiency | | hyperkinetic | | |
| | | syndrome | | and hypokinetic | | |
| | | (DTDS) | | movement | | |
| | | | | disorder^{152,154} | | |

### Allelic Variation in Genetic Subtypes

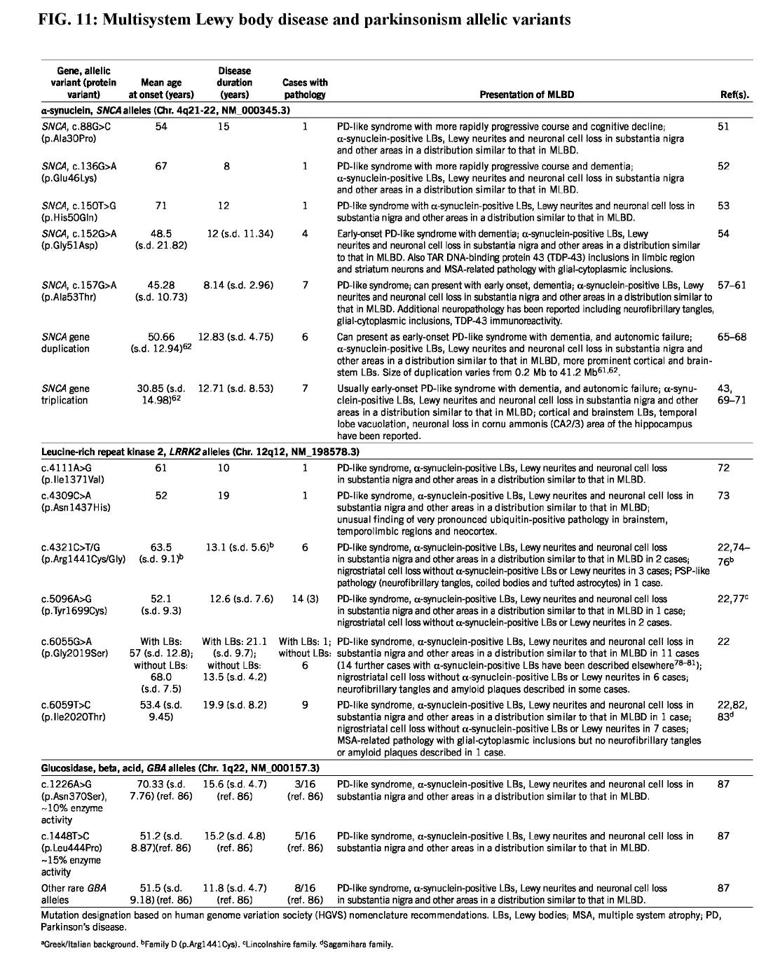

So far, all genetic forms of MLBD have shown parkinsonian features, whereas sporadic MLBD can present with a wide variety of pre- and nonmotor symptoms (21). Also, the same mutation (for example, NM_198578.3 (LRRK2): c.6055G>A (p.Gly2019Ser)) can cause MLBD in some instances, while in others resulting in degeneration limited to the substantia nigra and locus coeruleus with no Lewy body pathology (22). For three of the genes with well-established associations with MLBD, there are allelic differences in phenotype as well as differences that cannot be attributed to allelic variation (TABLE 2 and FIG. 11). Sorting these out clinically could be difficult, but, in some embodiments, assessment of the peripheral autonomic system can differentiate between MLBD and non-Lewy body parkinsonism, including the atypical parkinsonism (23). It is critical to understand this clinically, as peripheral nervous system involvement can be determined in the clinic, and affected individuals who lack peripheral nervous system involvement may have a different disease course and require different treatments from those who do not.

TABLE 2 (see also FIG. 11) shows Multisystem Lewy body disease and parkinsonism allelic variants for three of the genes with well-established associations with MLBD, i.e., SNCA, LRRK2, and GBA. There are allelic differences in phenotype as well as differences that cannot be attributed to allelic variation. Information analyzed included gene, allelic variant, mean age of onset, disease duration, cases with pathology, and presentation of MLBD.

TABLE 2
Multisystem Lewy body disease and parkinsonism allelic variants
Alpha-synuclein, SNC4 alleles (chr4q21-22, NM_000345.3)

| Gene, allelic | Age at onset | Disease | Number of | | |
|---|---|---|---|---|---|
| variant | (AAO) | duration | cases | Presentation of MLBD | Reference |
| 1. SNCA, c.88G > C | 60 yrs (SD 11) | 4 yrs | n = 5 | PD-like syndrome with more rapidly progressive | 51 |
| (p.Ala30Pro) | | | pathology | course and cognitive decline; alpha-synuclein positive | |
| | | | n = 1 | Lewy bodies, Lewy neurites and neuronal cells loss in | |
| | | | | the substantia nigra and other areas in a distribution | |
| | | | | similar to that seen in MLBD. | |
| 2. SNCA, c.136G > A | 60 yrs (SD 7) | 6 yrs, 15 yrs | n = 4, | PD-like syndrome with more rapidly progressive | 52 |
| (p.Glu46Lys) | | | pathology | course and cognitive decline; alpha-synuclein positive | |
| | | | n = 1 | Lewy bodies, Lewy neurites and neuronal cells loss in | |
| | | | | the substantia nigra and other areas in a distribution | |
| | | | | similar to that seen in MLBD. | |
| 3. SNCA, c.150T > G | 73 | 9 yrs | n = 2 | PD-like syndrome with alpha-synuclein positive Lewy | 53 |
| (p.His50Gln) | | | pathology | bodies, Lewy neurites and neuronal cells loss in the | |
| | | | n = 1 | substantia nigra and other areas in a distribution | |
| | | | | similar to that seen in MLBD. | |
| 4. SNCA, c,152G > A | 19 | 29 yrs | pathology | Early onset PD-like syndrome with dementia; alpha- | 54 |
| (p.GlySlAsp) | | | n = 1 | synuclein positive Lewy bodies, Lewy neurites and | |
| | | | | neuronal cells loss in the substantia nigra and other | |
| | | | | areas in a distribution similar to that seen in MLBD. | |
| | | | | Also TAR DNA-binding protein 43 (TDP-43) | |
| | | | | inclusions in limbic region and striatum neurons and | |
| | | | | multiple system atrophy-related pathology with glial- | |
| | | | | cytoplasmic inclusions. | |
| 5. SNCA, c.157G > A | 47 yrs (SD 12) | 8 yrs (SD 4) | n = 70 | PD-like syndrome, can present with early onset, | 55-59 |
| (p.Ala53Thr) | | | (Greek/Italian | dementia; alpha-synuclein positive Lewy bodies, | |
| | | | background), | Lewy neurites and neuronal cells loss in the substantia | |
| | | | pathology | nigra and other areas in a distribution similar to that | |
| | | | n = 8 | seen in MLBD. Additional neuropathology has been | |
| | | | | reported including neurofibrillary tangles, glial- | |
| | | | | cytoplasmic inclusions, TDP-43 immunoreactivity. | |
| 6. SNCA gene | 50 yrs (SD 11)⁶⁰ | 15 yrs (SD 6) | pathology | Can present as early onset PD-like syndrome with | 63-66 |
| duplication | | | n = 5 | dementia, and autonomic failure; alpha-synuclein | |
| | | | | positive Lewy bodies, Lewy neurites and neuronal | |
| | | | | cells loss in the substantia nigra and other areas in a | |
| | | | | distribution similar to that seen in MLBD, more | |
| | | | | prominent cortical and brainstem Lewy bodies. Size | |
| | | | | of duplication varies from 0.2 Mb to 41.2 Mb^{61, 62}. | |
| 7. SNCA gene | 40 yrs (SD 14)⁶⁰ | 7 yrs (SD 2) | pathology | Usually early onset PD-like syndrome with dementia, | 43, |
| triplication | | | n = 4 | and autonomic failure; alpha-synuclein positive Lewy | 67-69 |
| | | | | bodies, Lewy neurites and neuronal cells loss in the | |
| | | | | substantia nigra and other areas in a distribution | |
| | | | | similar to that seen in MLBD; cortical and brainstem | |
| | | | | Lewy bodies, temporal lobe vacuolation, neuronal | |
| | | | | loss in the cornu ammonis (CA2/3) area of the | |
| | | | | hippocampus has been reported. | |

| | | | | | |
|---|---|---|---|---|---|
| Gene, allelic | | Disease | Number | | |
| variant | Age at onset | duration | of cases | Presentation of MLBD | Reference |

Leucine-rich repeat kinase 2, LRRK2 alleles (chr12q12, NM_198578.3)

| | | | | | |
|---|---|---|---|---|---|
| c.4111A > G | 61 yrs | 10 yrs | pathology | PD-like syndrome, alpha-synuclein positive Lewy | 70 |
| (p.Ile1371Va1) | | | n = 1 | bodies, Lewy neurites and neuronal cells loss in the | |
| | | | | substantia nigra and other areas in a distribution | |
| | | | | similar to that seen in MLBD | |
| c.4309C > A | 52 yrs | 19 yrs | pathology | PD-like syndrome, alpha-synuclein positive Lewy | 71 |
| (p.Asn1437His) | | | n = 1 | bodies, Lewy neurites and neuronal cells loss in the | |
| | | | | substantia nigra and other areas in a distribution | |
| | | | | similar to that seen in MLBD, unusual finding of | |
| | | | | very pronounced ubiquitin-positive pathology in the | |
| | | | | brainstem, temporolimbic regions and neocortex. | |
| c.4321C > T/G | 63.5 yrs (SD 9.1) | 13.1 yrs (SD 5.6) | pathology | PD-like syndrome, alpha-synuclein positive Lewy | 22, 72 |
| (p.Arg1441Cys/Gly) | in family D | in family D | n = 6 | bodies, Lewy neurites and neuronal cells loss in the | Family D |
| | | | | substantia nigra and other areas in a distribution | (p.Arg1441Cys)^{73, 74} |
| | | | | similar to that seen in MLBD in 2 cases, | |
| | | | | nigrostriatal cell loss without alpha-synuclein | |
| | | | | positive Lewy bodies, Lewy neurites in 3 cases, 1 | |
| | | | | case with PSP-like pathology (neurofibrillary | |
| | | | | tangles, coiled bodies and tufted astrocytes) | |
| c.5096A > G | Mean 52.1 yrs | Mean 12.6 yrs, | total | PD-like syndrome, alpha-synuclein positive Lewy | 22, 75 |
| (p.Tyr1699Cys) | (SD 9.3) | SD 7.6 yrs | n = 14 | bodies, Lewy neurites and neuronal cells loss in the | Lincolnshire |
| | | | pathology | substantia nigra and other areas in a distribution | family |
| | | | n = 3 | similar to that seen in MLBD in 1 case, nigrostriatal | |
| | | | | cell loss without alpha-synuclein positive Lewy | |
| | | | | bodies, Lewy neurites in 2 cases. | |
| c.6055G > A | Mean 57 yrs | Mean 21.1 yrs | pathology | PD-like syndrome, alpha-synuclein positive Lewy | 22 |
| (p.Gly2019Ser) | (SD 12.8) with | (SD 9.7) with | n = 11 | bodies, Lewy neurites and neuronal cells loss in the | |
| | LBs, mean 68.0 | LB; 13.5 yrs | (with LB), | substantia nigra and other areas in a distribution | |
| | (SD7.5) w/o LBs | (SD 4.2) w/o LB | n = 6 | similar to that seen in MLBD in 11 cases and | |
| | | | (w/o LB) | furthermore, 14 cases with alpha-synuclein positive | |
| | | | | Lewy bodies pathology have been described | |
| | | | | elsewhere⁷⁶⁻⁷⁶, nigrostriatal cell loss without alpha- | |
| | | | | synuclein positive Lewy bodies, Lewy neurites in 6 | |
| | | | | cases, neurofibrillary tangles and amyloid plaques | |
| | | | | described in some cases. | |
| c.6059T > C | Mean 53.4 yrs | Mean 19.9 yrs, | pathology | PD-like syndrome, alpha-synuclein positive Lewy | 22, 80, 81 |
| (p.Ile2020Thr) | (SD 9.45) | (SD 8.2) | n = 9 | bodies, Lewy neurites and neuronal cells loss in the | Sagamihara |
| | | | | substantia nigra and other areas in a distribution | family |
| | | | | similar to that seen in MLBD in 1 case, nigrostriatal | |
| | | | | cell loss without alpha-synuclein positive Lewy | |
| | | | | bodies, Lewy neurites in 7 cases, no neurofibrillary | |
| | | | | tangles and amyloid plaques described, multiple | |
| | | | | system atrophy-related pathology with glial- | |
| | | | | cytoplasmic inclusions in 1 case. | |

Glucosidase, beta, acid, GBA, chr1q22, NM_000157.3

| | | | | | |
|---|---|---|---|---|---|
| c.1226A > G | Mean 59.39 | Mean 13.6 yrs | n = 16⁸⁴ | PD-like syndrome, alpha-synuclein positive Lewy | 85 |
| (p.Asn370Ser), ~10% | (SEM 2.1)^{82 83} | (SD 4.8)⁸⁴ | | bodies, Lewy neurites and neuronal cells loss in the | |
| enzyme activity | | | | substantia nigra and other areas in a distribution | |
| | | | | similar to that seen in MLBD. | |
| c.1448T > C | Mean 59.39 | Mean 13.6 yrs | n = 16⁸⁴ | PD-like syndrome, alpha-synuclein positive Lewy | 85 |
| (p.Leu444Pro) ~15% | (SEM 2.1)^{82 83} | (SD 4.8)⁸⁴ | | bodies, Lewy neurites and neuronal cells loss in the | |
| enzyme activity | | | | substantia nigra and other areas in a distribution | |
| | | | | similar to that seen in MLBD. | |
| Other rare GBA | Mean 59.39 | Mean 13.6 yrs | n = 16⁸⁴ | PD-like syndrome, alpha-synuclein positive Lewy | 85 |
| alleles | (SEM 2.1)^{82 83} | (SD 4.8)⁸⁴ | | bodies, Lewy neurites and neuronal cells loss in the | |
| | | | | substantia nigra and other areas in a distribution | |
| | | | | similar to that seen in MLBD. | |

Footnote:
Mutation designation based on human genome variation society (HGVS) nomenclature recommendations

### Analysis of Gene-Environment Interactions

Over the last quarter century, with a renaissance of research on the environmental determinants of typical Lewy body parkinsonism—that is, MLBD—and the explosion of genetics in the study of MLBD and other genes that cause other diseases that include parkinsonian symptoms, one might have assumed that these two disciplines would have worked closely together to unravel these disorders. However, epidemiologic studies are typically long and very expensive, and the causal possibilities are nearly infinite. On the other hand, the huge technical advances in genetics have accelerated genetic research in the Parkinson's field over the last 15 years. There are challenges that make it difficult for the two disciplines to integrate in a meaningful way. For example, a PubMed search for the terms “gene-environment” and “parkinsonism” netted only 64 references, whereas “parkinsonism” and “environment” netted slightly over 1,000 references, and “genetic” and “parkinsonism” over 8,000 references. This is particularly surprising given that most researchers believe that gene-environment (G×E) interaction will be a key to solving Parkinson's disease—yet there is a poverty of research in this area as compared to genetics.

The solution is for epidemiology and genetics to collaborate from the design of experiments onward (24, 25). For example, examining an epidemiologically characterized cohort on a genetic basis showed that working with the herbicide paraquat doubled the risk of Parkinson's disease, but the risk was increased 11-fold in subjects who also had a common genetic variant (a defective GSTT1 gene), representing one of the largest increases in risk for Parkinson's disease reported to date (26). Obstacles to be anticipated in this field relate to the number of subjects required for the studies, given that G×E effects for common variants are anticipated to be small and cohorts of hereditary mutation carriers will be limited by their rarity.

### Limits and Prospects of Stem Cell Genetic Models

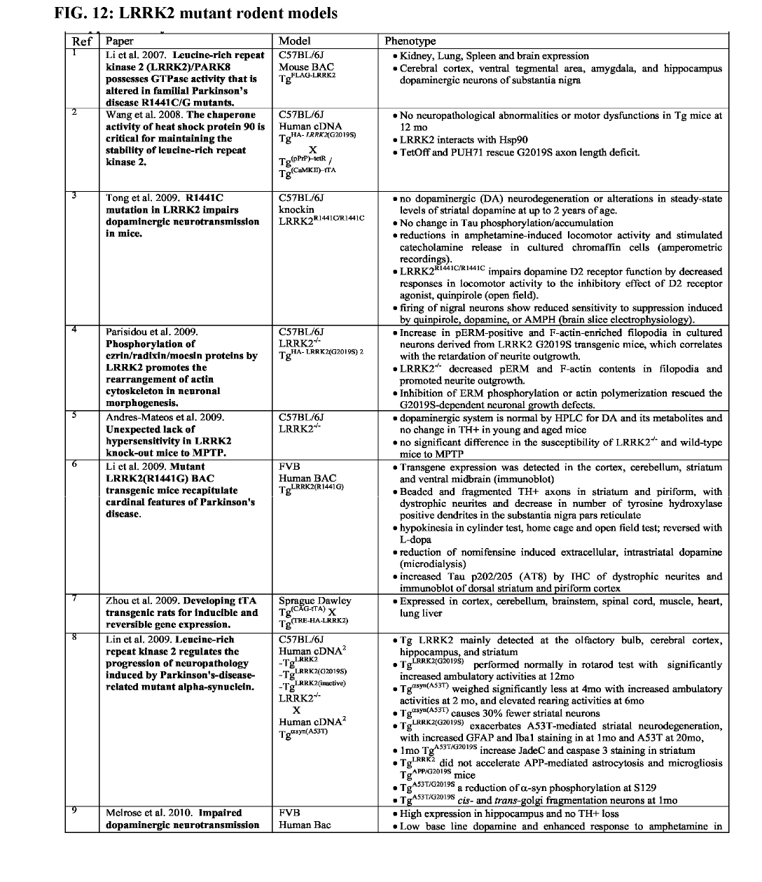

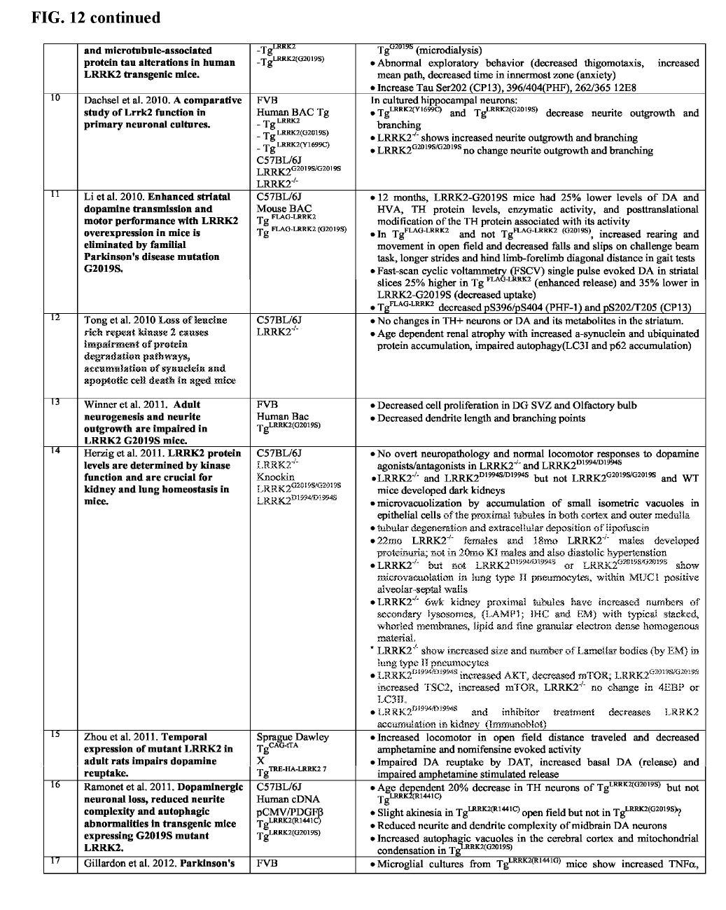

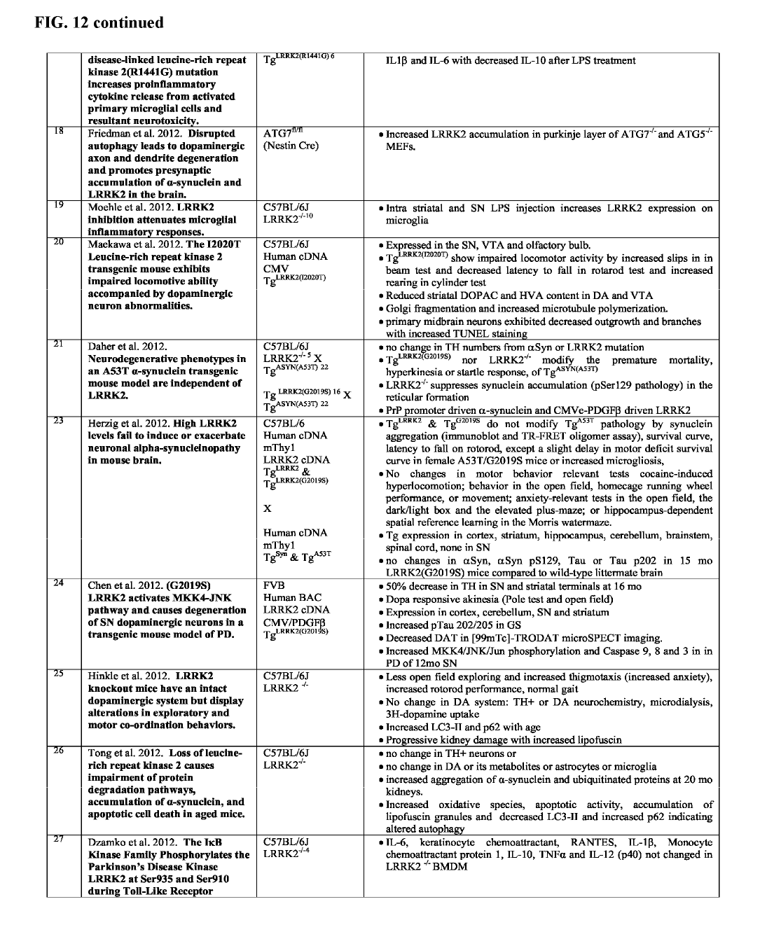

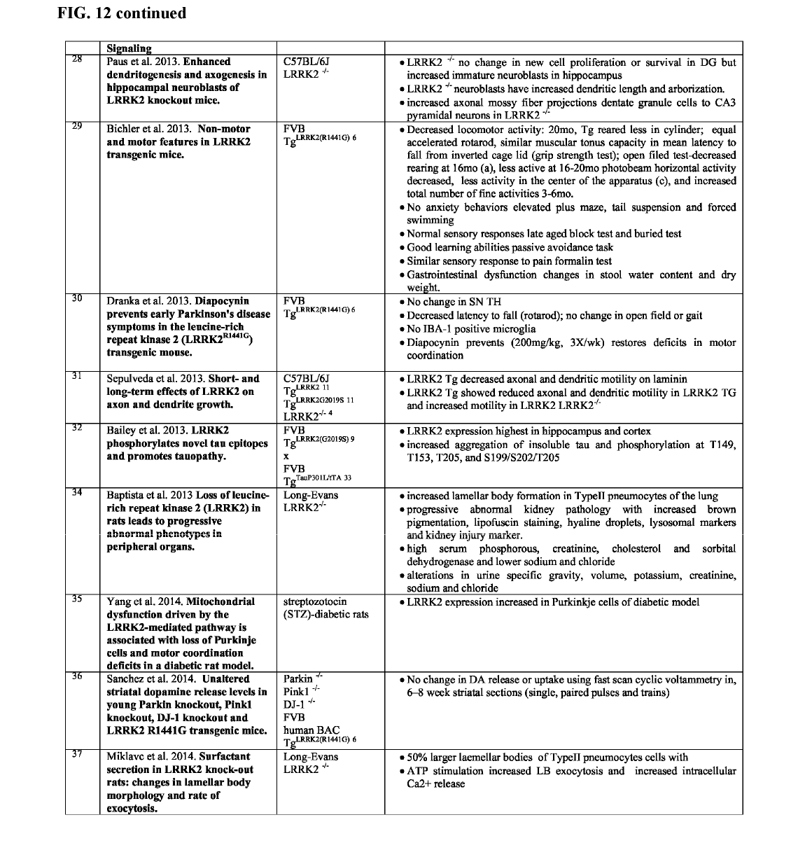

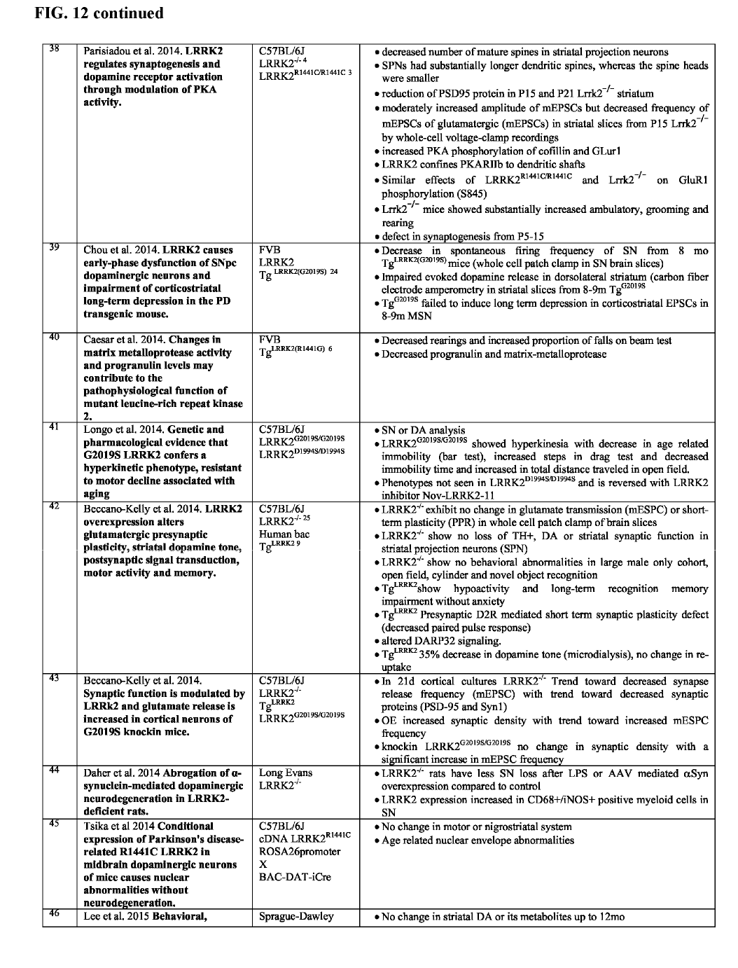

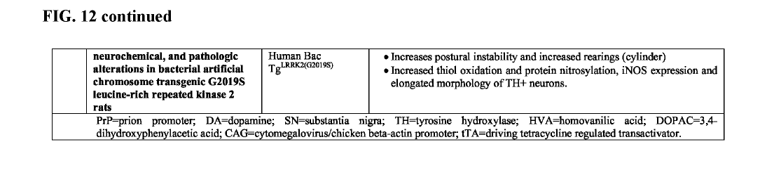

Although the MPTP model has proved highly useful in regard to testing new drugs for symptomatic therapy for parkinsonian signs and symptoms and agents that block the side-effects of L-dopa (27, 28), it is not a model of MLBD, and it has not proved useful for the discovery of drugs aimed at modifying disease progress. Of course the genetic discoveries have given birth to a myriad of transgenic models, but although these have been helpful, none appear to replicate the features of MLBD, and so their usefulness remains unclear when it comes to identifying disease-modifying agents that would be effective in the human sporadic genetic forms of the disease. This is the case even when transgenic and knockout technologies have been used to generate rodent versions carrying mutations that are identical to the human mutation (TABLE 3 and FIG. 12).

TABLE 3 (see also FIG. 12) provides exemplary LRRK2 mutant rodent models and their, phenotypes. In some embodiments, the LRRK2 mutant rodent model FVB Tg^{LRRK2(R1441G)}, as disclosed by Bichler et al. in 2013, presents with GI dysfunction with changes in stool water content and dry weight.

| Ref | Paper | Model | Phenotype |
|---|---|---|---|
| 1 | Li et al. 2007. Leucine- | C57BL/6J | Kidney, Lung, Spleen and brain expression |
| | rich repeat kinase 2 | Mouse BAC | Cerebral cortex, ventral tegmental area, |
| | (LRRK2)/PARK8 | Tg^{FLAG-LRRK2} | amygdala, and hippocampus dopaminergic neurons of |
| | possesses GTPase activity | | substantia nigra |
| | that is altered in familial | | |
| | Parkinson's disease | | |
| | R1441C/G mutants. | | |
| 2 | Wang et al. 2008. The | C57BL/6J | No neuropathological abnormalities or motor |
| | chaperone activity of | Human cDNA | dysfunctions in Tg mice at 12 mo |
| | heat shock protein 90 is | Tg^{HA-LRRK2(G2019S)} | LRRK2 interacts with Hsp90 |
| | critical for maintaining | X | TetOff and PUH71 rescue G2019S axon length |
| | the stability of leucine- | Tg^{(pPrP)-tetR}/ | deficit. |
| | rich repeat kinase 2. | Tg^{(CaMKII)-tTA} | |
| 3 | Tong et al. 2009. R1441C | C57BL/6J | no dopaminergic (DA) neurodegeneration or |
| | mutation in LRRK2 | knockin | alterations in steady-state levels of striatal dopamine at |
| | impairs dopaminergic | LRRK2^{R1441C/R1441C} | up to 2 years of age. |
| | neurotransmission in | | No change in Tau phosphorylation/accumulation |
| | mice. | | reductions in amphetamine-induced locomotor |
| | | | activity and stimulated catecholamine release in |
| | | | cultured chromaffin cells (amperometric recordings). |
| | | | LRRK2^{R1441C/R1441C} impairs dopamine D2 |
| | | | receptor function by decreased responses in locomotor |
| | | | activity to the inhibitory effect of D2 receptor agonist, |
| | | | quinpirole (open field). |
| | | | firing of nigral neurons show reduced sensitivity |
| | | | to suppression induced by quinpirole, dopamine, or |
| | | | AMPH (brain slice electrophysiology). |
| 4 | Parisidou et al. 2009. | C57BL/6J | Increase in pERM-positive and F-actin-enriched |
| | Phosphorylation of | LRRK2^{-/-} | filopodia in cultured neurons derived from LRRK2 |
| | ezrin/radixin/moesin | Tg^{HA-LRRK2(G2019S) 2} | G2019S transgenic mice, which correlates with the |
| | proteins by LRRK2 | | retardation of neurite outgrowth. |
| | promotes the | | LRRK2^{-/-} decreased pERM and F-actin contents |
| | rearrangement of actin | | in filopodia and promoted neurite outgrowth. |
| | cytoskeleton in neuronal | | Inhibition of ERM phosphorylation or actin |
| | morphogenesis. | | polymerization rescued the G2019S-dependent |
| | | | neuronal growth defects. |
| 5 | Andres-Mateos et al. 2009. | C57BL/6J | dopaminergic system is normal by HPLC for DA |
| | Unexpected lack of | LRRK2^{-/-} | and its metabolites and no change in TH+ in young |
| | hypersensitivity in | | and aged mice |
| | LRRK2 knock-out mice | | no significant difference in the susceptibility of |
| | to MPTP. | | LRRK2^{-/-} and wild-type mice to MPTP |
| 6 | Li et al. 2009. Mutant | FVB | Transgene expression was detected in the cortex, |
| | LRRK2(R1441G) BAC | Human BAC | cerebellum, striatum and ventral midbrain |
| | transgenic mice | Tg^{LRRK2(R1441G)} | (immunoblot) |
| | recapitulate cardinal | | Beaded and fragmented TH+ axons in striatum |
| | features of Parkinson's | | and piriform, with dystrophic neurites and decrease in |
| | disease. | | number of tyrosine hydroxylase positive dendrites in |
| | | | the substantia nigra pars reticulate |
| | | | hypokinesia in cylinder test, home cage and open |
| | | | field test; reversed with L-dopa |
| | | | reduction of nomifensine induced extracellular, |
| | | | intrastriatal dopamine (microdialysis) |
| | | | increased Tau p202/205 (AT8) by IHC of |
| | | | dystrophic neurites and immunoblot of dorsal striatum |
| | | | and piriform cortex |
| 7 | Zhou et al. 2009. | Sprague | Expressed in cortex, cerebellum, brainstem, spinal |
| | Developing tTA | Dawley | cord, muscle, heart, lung liver |
| | transgenic rats for | Tg^{(CAG-tTA)} | |
| | inducible and reversible | X | |
| | gene expression. | Tg^{(TRE-HA-LRRK2)} | |
| 8 | Lin et al. 2009. Leucine- | C57BL/6J | Tg LRRK2 mainly detected at the olfactory bulb, |
| | rich repeat kinase 2 | Human | cerebral cortex, hippocampus, and striatum |
| | regulates the progression | cDNA² | Tg^{LRRK2(G5019S)} performed normally in rotarod |
| | of neuropathology | Tg^{LRRK2} | test with significantly increased ambulatory activities |
| | induced by Parkinson's- | Tg^{LRRK2(G2019S)} | at 12 mo |
| | disease-related mutant | Tg^{LRRK2(inactive)} | Tg^{αsyn(A53T)} weighed significantly less at 4 mo with |
| | alpha-synuclein. | TRRK2^{-/-} | increased ambulatory activities at 2 mo, and elevated |
| | | X | rearing activities at 6 mo |
| | | Human cDNA² | Tg^{αsyn(A53T)} causes 30% fewer striatal neurons |
| | | Tg^{αsyn(A53T)} | Tg^{LRRK2(G2019S)} exacerbates A53T-mediated |
| | | | striatal neurodegeneration, with increased GFAP and |
| | | | Iba1 staining in at 1 mo and A53T at 20 mo, |
| | | | 1 mo Tg^{A53T/G2019S} increase JadeC and caspase 3 |
| | | | staining in striatum |
| | | | Tg^{LRRK2} did not accelerate APP-mediated |
| | | | astrocytosis and microghosis Tg^{APP/G2019S} mice |
| | | | Tg^{A53T/G2019S} a reduction of α-syn |
| | | | phosphorylation at S129 |
| | | | Tg^{A53T/G2019S} cis- and trans-golgi fragmentation |
| | | | neurons at 1 mo |
| 9 | Melrose et al. 2010. | FVB | High expression in hippocampus and no TH+ loss |
| | Impaired dopaminergic | Human Bac | Low base line dopamine and enhanced response |
| | neurotransmission and | Tg^{LRRK2} | to amphetamine in Tg^{G2019S} (microdialysis) |
| | microtubule-associated | Tg^{LRRK2(G2019S)} | Abnormal exploratory behavior (decreased |
| | protein tau alterations in | | thigomotaxis, increased mean path, decreased time in |
| | human LRRK2 | | innermost zone (anxiety) |
| | transgenic mice. | | Increase Tau Ser202 (CP13), 396/404(PHF), |
| | | | 262/365 12E8 |
| 10 | Dachsel et al. 2010. A | FVB | In cultured hippocampal neurons: |
| | comparative study of | Human BAC | Tg^{LRRK2(Y1699C)} and Tg^{LRRK2(G2019S)} decrease |
| | Lrrk2 function in | Tg | neurite outgrowth and branching |
| | primary neuronal | Tg^{LRRK2} | LRRK2^{-/-} shows increased neurite outgrowth and |
| | cultures. | Tg^{LRRK2(G2019S)} | branching |
| | | Tg^{LRRK2(Y1699C)} | LRRK2^{G2019S/G2019S} no change neurite outgrowth |
| | | C57BL/6J | and branching |
| | | LRRK2^{G2019S/G2019S} | |
| | | LRRK2^{-/-} | |
| 11 | Li et al. 2010. Enhanced | C57BL/6J | 12 months, LRRK2-G2019S mice had 25% lower |
| | striatal dopamine | Mouse BAC | levels of DA and HVA, TH protein levels, enzymatic |
| | transmission and motor | Tg^{FLAG-LRRK2} | activity, and posttranslational modification of the TH |
| | performance with | Tg^{FLAG-LRRK2 (G2019S)} | protein associated with its activity |
| | LRRK2 overexpression | | In Tg^{FLAG-LRRK2} and not Tg^{FLAG-LRRK2 (G2019S)}, |
| | in mice is eliminated by | | increased rearing and movement in open field and |
| | familial Parkinson's | | decreased falls and slips on challenge beam task, |
| | disease mutation G2019S. | | longer strides and hind limb-forelimb diagonal |
| | | | distance in gait tests |
| | | | Fast-scan cyclic voltammetry (FSCV) single |
| | | | pulse evoked DA in striatal slices 25% higher in |
| | | | Tg^{FLAG-LRRK2} (enhanced release) and 35% lower in |
| | | | LRRK2-G2019S (decreased uptake) |
| | | | Tg^{FLAG-LRRK2} decreased pS396/pS404 (PHF-1) |
| | | | and pS202/T205 (CP13) |
| 12 | Tong et al. 2010 Loss of | C57BL/6J | No changes in TH+ neurons or DA and its |
| | leucine rich repeat kinase | LRRK2^{-/-} | metabolites in the striatum. |
| | 2 causes impairment of | | Age dependent renal atrophy with increased a- |
| | protein degradation | | synuclein and ubiquinated protein accumulation, |
| | pathways, accumulation | | impaired autophagy(LC3I and p62 accumulation) |
| | of synuclein and | | |
| | apoptotic cell death in | | |
| | aged mice | | |
| 13 | Winner et al. 2011. Adult | FVB | Decreased cell proliferation in DG SVZ and |
| | neurogenesis and neurite | Human Bac | Olfactory bulb |
| | outgrowth are impaired | Tg^{LRRK2(G2019S}) | Decreased dendrite length and branching points |
| | in LRRK2 G2019S mice. | | |
| 14 | Herzig et al. 2011. | C57BL/6J | No overt neuropathology and normal locomotor |
| | LRRK2 protein levels are | LRRK2^{-/-} | responses to dopamine agonists/antagonists in LRRK2^{-/-} |
| | determined by kinase | Knockin | and LRRK2^{D1994/D1994S} |
| | function and are crucial | LRRK2^{G2019S/G2019S} | LRRK2^{-/-} and LRRK2^{D1994S/D1994S} but not |
| | for kidney and lung | LRRK2^{D1994/D1994S} | LRRK2^{G2019S/G2019S} and WT mice developed dark |
| | homeostasis in mice. | | kidneys |
| | | | microvacuolization by accumulation of small |
| | | | isometric vacuoles in epithelial cells of the proximal |
| | | | tubules in both cortex and outer medulla |
| | | | tubular degeneration and extracellular deposition |
| | | | of lipofuscin |
| | | | 22 mo LRRK2^{-/-} females and 18 mo LRRK2^{-/-} |
| | | | males developed proteinuria; not in 20 mo KI males |
| | | | and also diastolic hypertenstion |
| | | | LRRK2^{-/-} but not LRRK2^{D1994/D1994S} or |
| | | | LRRK2^{G2019S/G2019S} show microvacuolation in lung |
| | | | type II pneumocytes, within MUC1 positive alveolar- |
| | | | septal walls |
| | | | LRRK2^{-/-} 6 wk kidney proximal tubules have |
| | | | increased numbers of secondary lysosomes, (LAMP1; |
| | | | IHC and EM) with typical stacked, whorled |
| | | | membranes, lipid and fine granular electron dense |
| | | | homogenous material. |
| | | | LRRK2^{-/-} show increased size and number of Lamellar |
| | | | bodies (by EM) in lung type II pneumocytes |
| | | | LRRK2^{D1994/D1994S} increased AKT, decreased |
| | | | mTOR; LRRK2^{G2019S/G2019S} increased TSC2, increased |
| | | | mTOR, LRRK2^{-/-} no change in 4EBP or LC3II. |
| | | | LRRK2^{D1994/D1994S} and inhibitor treatment |
| | | | decreases LRRK2 accumulation in kidney |
| | | | (Immunoblot) |
| 15 | Zhou et al. 2011. | Sprague | Increased locomotor in open field distance |
| | Temporal expression of | Dawley | traveled and decreased amphetamine and nomifensine |
| | mutant LRRK2 in adult | Tg^{CAG-tTA} | evoked activity |
| | rats impairs dopamine | X | Impaired DA reuptake by DAT, increased basal |
| | reuptake. | Tg^{TRE-HA-LRRK2 7} | DA (release) and impaired amphetamine stimulated |
| | | | release |
| 16 | Ramonet et al. 2011. | C57BL/6J | Age dependent 20% decrease in TH neurons of |
| | Dopaminergic neuronal | Human cDNA | Tg^{LRRK2(G2019S)} but not Tg^{LRRK2(R1441C)} |
| | loss, reduced neurite | pCMV/PDGFβ | Slight akinesia in Tg^{LRRK2(R1441C)} open field but |
| | complexity and | Tg^{LRRK2(R1441C)} | not in Tg^{LRRK2(G2019S)}? |
| | autophagic abnormalities | Tg^{LRRK2(G2019S)} | Reduced neurite and dendrite complexity of |
| | in transgenic mice | | midbrain DA neurons |
| | expressing G2019S | | Increased autophagic vacuoles in the cerebral |
| | mutant LRRK2. | | cortex and mitochondrial condensation in |
| | | | Tg^{LRRK2(G2019S)} |
| 17 | Gillardon et al. 2012. | FVB | Microglial cultures from Tg^{LRRK2(R1441G)} mice |
| | Parkinson's disease- | Tg^{LRRK2(R1441G) 6} | show increased TNFα, IL1β and IL-6 with decreased |
| | linked leucine-rich repeat | | IL-10 after LPS treatment |
| | kinase 2(R1441G) | | |
| | mutation increases | | |
| | proinflammatory | | |
| | cytokine release from | | |
| | activated primary | | |
| | microglial cells and | | |
| | resultant neurotoxicity. | | |
| 18 | Friedman et al. 2012. | ATG7^{fl/fl} | Increased LRRK2 accumulation in purkinje layer |
| | Disrupted autophagy | (Nestin Cre) | of ATG7^{-/-} and ATG5^{-/-} MEFs. |
| | leads to dopaminergic | | |
| | axon and dendrite | | |
| | degeneration and | | |
| | promotes presynaptic | | |
| | accumulation of α- | | |
| | synuclein and LRRK2 in | | |
| | the brain. | | |
| 19 | Moehle et al. 2012. | C57BL/6J | Intra striatal and SN LPS injection increases |
| | LRRK2 inhibition | LRRK2^{-/-10} | LRRK2 expression on microglia |
| | attenuates microglial | | |
| | inflammatory responses. | | |
| 20 | Maekawa et al. 2012. The | C57BL/6J | Expressed in the SN, VTA and olfactory bulb. |
| | I2020T Leucine-rich | Human cDNA | Tg^{LRRK2(I2020T)} show impaired locomotor activity |
| | repeat kinase 2 | CMV | by increased slips in in beam test and decreased |
| | transgenic mouse | Tg^{LRRK2(I2020T)} | latency to fall in rotarod test and increased rearing in |
| | exhibits impaired | | cylinder test |
| | locomotive ability | | Reduced striatal DOPAC and HVA content in DA |
| | accompanied by | | and VTA |
| | dopaminergic neuron | | Golgi fragmentation and increased microtubule |
| | abnormalities. | | polymerization. |
| | | | primary midbrain neurons exhibited decreased |
| | | | outgrowth and branches |
| | | | with increased TUNEL staining |
| 21 | Daher et al. 2012. | C57BL/6J | no change in TH numbers from αSyn or LRRK2 |
| | Neurodegenerative | LRRK2^{-/-5} | mutation |
| | phenotypes in an A53T | X | Tg^{LRRK2(G2019S)} nor LRRK2^{-/-} modify the |
| | α-synuclein transgenic | Tg^{ASYN(A53T) 22} | premature mortality, hyperkinesia or startle response, |
| | mouse model are | Tg^{LRRK2(G2019S) 16} | ofTg^{ASYN(A53T)} |
| | independent of LRRK2. | X | LRRK2^{-/-} suppresses synuclein accumulation |
| | | Tg^{ASYN(A53T) 22} | (pSer129 pathology) in the reticular formation |
| | | | PrP promoter driven α-synuclein and CMVe- |
| | | | PDGFβ driven LRRK2 |
| 23 | Herzig et al. 2012. High | C57BL/6 | Tg^{LRRK2} & Tg^{G2019S} do modify Tg^{A53T} |
| | LRRK2 levels fail to | Human cDNA | pathology by synuclein aggregation (immunoblot and |
| | induce or exacerbate | mThy1 | TR-FRET oligomer assay), survival curve, latency to |
| | neuronal alpha- | LRRK2 cDNA | fall on rotorod, except a slight delay in motor deficit |
| | synucleinopathy in | Tg^{LRRK2} & | survival curve in female A53T/G2019S mice or |
| | mouse brain. | Tg^{LRRK2(G2019S)} | increased microgliosis, |
| | | X | No changes in motor behavior relevant tests |
| | | Human cDNA | cocaine-induced hyperlocomotion; behavior in the |
| | | mThy1 | open field, homecage running wheel performance, or |
| | | Tg^{Syn} & Tg^{A53T} | movement; anxiety-relevant tests in the open field, the |
| | | | dark/light box and the elevated plus-maze; or |
| | | | hippocampus-dependent spatial reference learning in |
| | | | the Morris watermaze. |
| | | | Tg expression in cortex, striatum, hippocampus, |
| | | | cerebellum, brainstem, spinal cord, none in SN |
| | | | no changes in αSyn, αSyn pS129, Tau or Tau |
| | | | p202 in 15 mo LRRK2(G2019S) mice compared to |
| | | | wild-type littermate brain |
| 24 | Chen et al. 2012. | FVB | 50% decrease in TH in SN and striatal terminals |
| | (G2019S) LRRK2 | Human BAC | at 16 mo |
| | activates MKK4-JNK | LRRK2 cDNA | Dopa responsive akinesia (Pole test and open |
| | pathway and causes | CMV/PDGFβ | field) |
| | degeneration of SN | Tg^{LRRK2(G2019S)} | Expression in cortex, cerebellum, SN and striatum |
| | dopaminergic neurons in | | Increased pTau 202/205 in GS |
| | a transgenic mouse | | Decreased DAT in [99mTc]-TRODAT |
| | model of PD. | | microSPECT imaging. |
| | | | Increased MKK4/JNK/Jun phosphorylation and |
| | | | Caspase 9, 8 and 3 in in PD of 12 mo SN |
| 25 | Hinkle et al. 2012. | C57BL/6J | Less open field exploring and increased |
| | LRRK2 knockout mice | LRRK2^{-/-} | thigmotaxis (increased anxiety), increased rotorod |
| | have an intact | | performance, normal gait |
| | dopaminergic system but | | No change in DA system: TH+ or DA |
| | display alterations in | | neurochemistry, micro dialysis, 3H-dopamine uptake |
| | exploratory and motor | | Increased LC3-II and p62 with age |
| | co-ordination behaviors. | | Progressive kidney damage with increased |
| | | | lipofuscin |
| 26 | Tong et al. 2012. Loss of | C57BL/6J | no change in TH+ neurons or |
| | leucine-rich repeat | LRRK2^{-/-} | no change in DA or its metabolites or astrocytes |
| | kinase 2 causes | | or microglia |
| | impairment of protein | | increased aggregation of α-synuclein and |
| | degradation pathways, | | ubiquitinated proteins at 20 mo kidneys. |
| | accumulation of α- | | Increased oxidative species, apoptotic activity, |
| | synuclein, and apoptotic | | accumulation of lipofuscin granules and decreased |
| | cell death in aged mice. | | LC3-II and increased p62 indicating altered autophagy |
| 27 | Dzamko et al. 2012. The | C57BL/6J | IL-6, keratinocyte chemoattractant, RANTES, IL- |
| | IκB Kinase Family | LRRK2^{-/-4} | 1β, Monocyte chemoattractant protein 1, IL-10, TNFα |
| | Phosphorylates the | | and IL-12 (p40) not changed in LRRK2^{-/-} BMDM |
| | Parkinson's Disease | | |
| | Kinase LRRK2 at Ser935 | | |
| | and Ser910 during Toll- | | |
| | Like Receptor Signaling | | |
| 28 | Paus et al. 2013. | C57BL/6J | LRRK2^{-/-} no change in new cell proliferation or |
| | Enhanced | LRRK2^{-/-} | survival in DG but increased immature neuroblasts in |
| | dendritogenesis and | | hippocampus |
| | axogenesis in | | LRRK2^{-/-} neuroblasts have increased dendritic |
| | hippocampal neuroblasts | | length and arborization. |
| | of LRRK2 knockout | | increased axonal mossy fiber projections dentate |
| | mice. | | granule cells to CA3 pyramidal neurons in LRRK2^{-/-} |
| 29 | Bichler et al. 2013. Non- | FVB | Decreased locomotor activity: 20 mo, Tg reared |
| | motor and motor | Tg^{KRRK2(R1441G) 6} | less in cylinder; equal accelerated rotarod, similar |
| | features in LRRK2 | | muscular tonus capacity in mean latency to fall from |
| | transgenic mice. | | inverted cage lid (grip strength test); open filed test- |
| | | | decreased rearing at 16 mo (a), less active at 16-20 mo |
| | | | photobeam horizontal activity decreased, less activity |
| | | | in the center of the apparatus (c), and increased total |
| | | | number of fine activities 3-6 mo. |
| | | | No anxiety behaviors elevated plus maze, tail |
| | | | suspension and forced swimming |
| | | | Normal sensory responses late aged block test and |
| | | | buried test |
| | | | Good learning abilities passive avoidance task |
| | | | Similar sensory response to pain formalin test |
| | | | Gastrointestinal dysfunction changes in stool |
| | | | water content and dry weight. |
| 30 | Dranka et al. 2013. | FVB | No change in SN TH |
| | Diapocynin prevents | Tg^{LRRK2(R1441G) 6} | Decreased latency to fall (rotarod); no change in |
| | early Parkinson's disease | | open field or gait |
| | symptoms in the leucine- | | No IBA-1 positive microglia |
| | rich repeat kinase 2 | | Diapocynin prevents (200 mg/kg, 3X/wk) restores |
| | (LRRK2R^{1441G}) | | deficits in motor coordination |
| | transgenic mouse. | | |
| 31 | Sepulveda et al. 2013. | C57BL/6J | LRRK2 Tg decreased axonal and dendritic |
| | Short- and long-term | Tg^{LRRK2 11} | motility on laminin |
| | effects of LRRK2 on | Tg^{LRRK2G2019S 11} | LRRK2 Tg showed reduced axonal and dendritic |
| | axon and dendrite | LRRK2^{-/-4} | motility in LRRK2 TG and increased motility in |
| | growth. | | LRRK2 LRRK2^{-/-} |
| 32 | Bailey et al. 2013. LRRK2 | FVB | LRRK2 expression highest in hippocampus and |
| | phosphorylates novel tau | Tg^{LRRK2(G2019S) 9} | cortex |
| | epitopes and promotes | x | increased aggregation of insoluble tau and |
| | tauopathy. | FVB | phosphorylation at T149, T153, T205, and |
| | | Tg^{TauP301L/tTA 33} | S199/S202/T205 |
| 34 | Baptista et al. 2013 Loss | Long-Evans | increased lamellar body formation in TypeII |
| | of leucine-rich repeat | LRRK2^{-/-} | pneumocytes of the lung |
| | kinase 2 (LRRK2) in rats | | progressive abnormal kidney pathology with |
| | leads to progressive | | increased brown pigmentation, lipofuscin staining, |
| | abnormal phenotypes in | | hyaline droplets, lysosomal markers and kidney injury |
| | peripheral organs. | | marker. |
| | | | high serum phosphorous, creatinine, cholesterol |
| | | | and sorbital dehydrogenase and lower sodium and |
| | | | chloride |
| | | | alterations in urine specific gravity, volume, |
| | | | potassium, creatinine, sodium and chloride |
| 35 | Yang et al. 2014. | streptozotocin | LRRK2 expression increased in Purkinkje cells of |
| | Mitochondrial | (STZ)-diabetic | diabetic model |
| | dysfunction driven by the | rats | |
| | LRRK2-mediated | | |
| | pathway is associated | | |
| | with loss of Purkinje cells | | |
| | and motor coordination | | |
| | deficits in a diabetic rat | | |
| | model. | | |
| 36 | Sanchez et al. 2014. | Parkin^{-/-} | No change in DA release or uptake using fast |
| | Unaltered striatal | Pink1^{-/-} | scan cyclic voltammetry in, 6-8 week striatal sections |
| | dopamine release levels | DJ-1^{-/-} | (single, paired pulses and trains) |
| | in young Parkin | FVB | |
| | knockout, Pink1 | human BAC | |
| | knockout, DJ-1 knockout | Tg^{LRRK2(R1441G) 6} | |
| | and LRRK2 R1441G | | |
| | transgenic mice. | | |
| 37 | Miklavc et al. 2014. | Long-Evans | 50% larger laemellar bodies of TypeII |
| | Surfactant secretion in | LRRK2^{-/-} | pneumocytes cells with |
| | LRRK2 knock-out rats: | | ATP stimulation increased LB exocytosis and |
| | changes in lamellar body | | increased intracellular Ca2+ release |
| | morphology and rate of | | |
| | exocytosis. | | |
| 38 | Parisiadou et al. 2014. | C57BL/6J | decreased number of mature spines in striatal |
| | LRRK2 regulates | LRRK2^{-/-4} | projection neurons |
| | synaptogenesis and | LRRK2^{R1441C/R1441C 3} | SPNs had substantially longer dendritic spines, |
| | dopamine receptor | | whereas the spine heads were smaller |
| | activation through | | reduction of PSD95 protein in P15 and P21 Lrrk2^{-/-} |
| | modulation of PKA | | striatum |
| | activity. | | moderately increased amplitude of mEPSCs but |
| | | | decreased frequency of mEPSCs of glutamatergic |
| | | | (mEPSCs) in striatal slices from P15 Lrrk2^{-/-} by |
| | | | whole-cell voltage-clamp recordings |
| | | | increased PKA phosphorylation of cofillin and GLur1 |
| | | | LRRK2 confines PKARIIb to dendritic shafts |
| | | | Similar effects of LRRK2^{R1441C/R1441C} and Lrrk2^{-/-} on |
| | | | GluR1 phosphorylation (S845) |
| | | | Lrrk2^{-/-} mice showed substantially increased |
| | | | ambulatory, grooming and rearing |
| | | | defect in synaptogenesis from P5-15 |
| 39 | Chou et al. 2014. LRRK2 | FVB | Decrease in spontaneous firing frequency of SN |
| | causes early-phase | LRRK2 | from 8 mo Tg^{LRRK2(G2019S)} (whole cell patch |
| | dysfunction of SNpc | Tg^{LRRK2(G2019S) 24} | clamp in SN brain slices) |
| | dopaminergic neurons | | Impaired evoked dopamine release in dorsolateral |
| | and | | striatum (carbon fiber electrode amperometry in |
| | impairment of | | striatal slices from 8-9 m Tg^{G2019S} |
| | corticostriatal long-term | | Tg^{G2019S} failed to induce long term depression in |
| | depression in the PD | | corticostriatal EPSCs in 8-9 m MSN |
| | transgenic mouse. | | |
| 40 | Caesar et al. 2014. | FVB | Decreased rearings and increased proportion of |
| | Changes in matrix | Tg^{LRRK2(R1441G) 6} | falls on beam test |
| | metalloprotease activity | | Decreased progranulin and matrix- |
| | and progranulin levels | | metalloprotease |
| | may contribute to the | | |
| | pathophysiological | | |
| | function of mutant | | |
| | leucine-rich repeat | | |
| | kinase 2. | | |
| 41 | Longo et al. 2014. Genetic | C57BL/6J | SN or DA analysis |
| | and pharmacological | LRRK2^{G2019S/G2019S} | LRRK2^{G2019S/G2019S} showed hyperkinesia with |
| | evidence that G2019S | LRRK2^{D1994S/D1994S} | decrease in age related immobility (bar test), increased |
| | LRRK2 confers a | | steps in drag test and decreased immobility time and |
| | hyperkinetic phenotype, | | increased in total distance traveled in open field. |
| | resistant to motor decline | | Phenotypes not seen in LRRK2^{D1994S/D1994S} and is |
| | associated with aging | | reversed with LRRK2 inhibitor Nov-LRRK2-11 |
| 42 | Beccano-Kelly et al. 2014. | C57BL/6J | LRRK2^{-/-} exhibit no change in glutamate |
| | LRRK2 overexpression | LRRK2^{-/-25} | transmission (mESPC) or short-term plasticity (PPR) |
| | alters glutamatergic | Human bac | in whole cell patch clamp of brain slices |
| | presynaptic plasticity, | Tg^{LRRK2 9} | LRRK2^{-/-} show no loss of TH+, DA or striatal |
| | striatal dopamine tone, | | synaptic function in striatal projection neurons (SPN) |
| | postsynaptic signal | | LRRK2^{-/-} show no behavioral abnormalities in |
| | transduction, motor | | large male only cohort, open field, cylinder and novel |
| | activity and memory. | | object recognition |
| | | | Tg^{LRRK2}show hypoactivity and long-term |
| | | | recognition memory impairment without anxiety |
| | | | Tg^{LRRK2} Presynaptic D2R mediated short term |
| | | | synaptic plasticity defect (decreased paired pulse |
| | | | response) |
| | | | altered DARP32 signaling. |
| | | | Tg^{LRRK2} 35% decrease in dopamine tone |
| | | | (microdialysis), no change in re-uptake |
| 43 | Beccano-Kelly et al. 2014. | C57BL/6J | In 21 d cortical cultures LRRK2^{-/-} Trend toward |
| | Synaptic function is | LRRK2^{-/-} | decreased synapse release frequency (mEPSC) with |
| | modulated by LRRk2 | Tg^{LRRK2} | trend toward decreased synaptic proteins (PSD-95 and |
| | and glutamate release is | LRRK2^{G2019S/G2019S} | Syn1) |
| | increased in cortical | | OE increased synaptic density with trend toward |
| | neurons of G2019S | | increased mESPC frequency |
| | knockin mice. | | knockin LRRK2^{G2019S/G2019S} no change in synaptic |
| | | | density with a significant increase in mEPSC |
| | | | frequency |
| 44 | Daher et al. 2014 | Long Evans | LRRK2^{-/-} rats have less SN loss after LPS or |
| | Abrogation of α- | LRRK2^{-/-} | AAV mediated αSyn overexpression compared to |
| | synuclein-mediated | | control |
| | dopaminergic | | LRRK2 expression increased in CD68+/iNOS+ |
| | neurodegeneration in | | positive myeloid cells in SN |
| | LRRK2-deficient rats. | | |
| 45 | Tsika et al 2014 | C57BL/6J | No change in motor or nigro striatal system |
| | Conditional expression of | cDNA | Age related nuclear envelope abnormalities |
| | Parkinson's disease- | LRRK2^{R1441C} | |
| | related R1441C LRRK2 | ROSA26promoter | |
| | in midbrain | X | |
| | dopaminergic neurons of | BAC-DAT- | |
| | mice causes nuclear | iCre | |
| | abnormalities without | | |
| | neurodegeneration. | | |
| 46 | Lee et al. 2015 | Sprague- | No change in striatal DA or its metabolites up to |
| | Behavioral, | Dawley | 12 mo |
| | neurochemical, and | Human Bac | Increases postural instability and increased |
| | pathologic alterations in | Tg^{LRRK2(G2019S)} | rearings (cylinder) |
| | bacterial artificial | | Increased thiol oxidation and protein |
| | chromosome transgenic | | nitrosylation, iNOS expression and elongated |
| | G2019S leucine-rich | | morphology of TH+ neurons. |
| | repeated kinase 2 rats | | |

1) Table as of February 2015.
2) pPrP = prion promoter; DA = dopamine; SN = substantia nigra; TH = tyrosine hydroxylase; HVA = homovanilic acid; DOPAC = 3,4-dihydroxyphenylacetic acid; CAG = cytomegalovirus/chicken beta-actin promoter; tTA = driving tetracycline regulated transactivator.

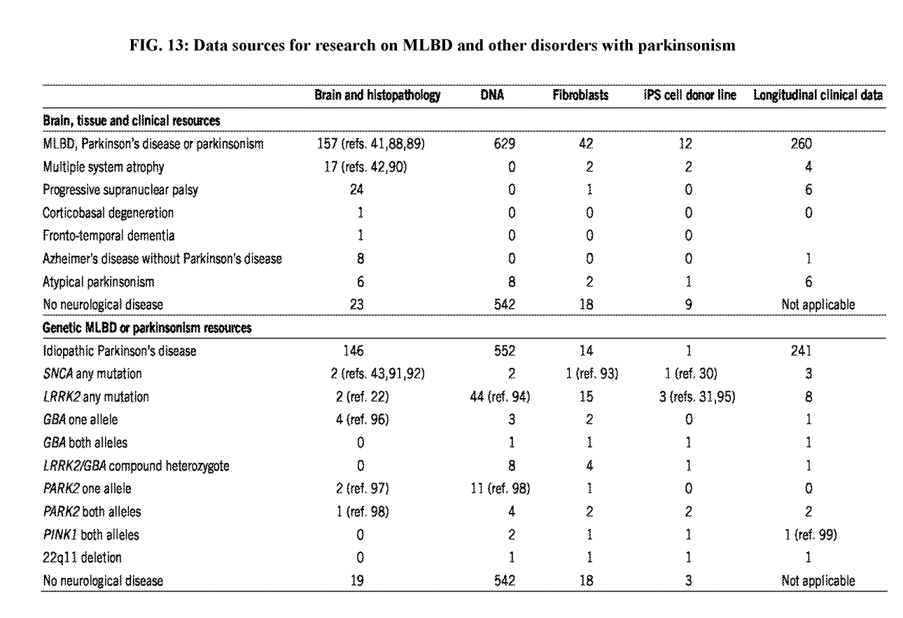

One option is to turn to human cell models that are based on genetic variations that cause disease in humans. This is feasible as many clinical centers and organizations have been collecting various biospecimens for use in both discovery and drug development along with the phenotypic information that can be obtained about the affected individual donating the tissue either from medical records or as part of a clinical study. In some embodiments, a research model that includes medical history, clinical evaluations, environmental risk exposure and biospecimens may be used, integrating and searching across multiple years of data, including collected tissues from subjects with clinical and neuropathological diagnoses of MLBD and parkinsonism due to a variety of causes (such as multiple system atrophy) and additional diseases that show a-synuclein- and/or tau-related neuropathology (TABLE 4, upper part; see also FIG. 13). Within these categories, samples from subjects with genetically causal forms of these disorders were collected in some embodiments of the invention (TABLE 4, lower part; see also FIG. 13).

TABLE 4 (see also FIG. 13) shows exemplary data resources for research on MLBD and other disorders with parkinsonism. Data sources are generally categorized as two types: 1) brain, tissue and clinical resources; and 2) genetic MLBD or parkinsonism resources. In some embodiments, tissues were collected from subjects with clinical and neuropathological diagnoses of MLBD and parkinsonism due to a variety of causes (such as multiple system atrophy) and additional diseases that show a-synuclein- and/or tau-related neuropathology (upper part of TABLE 4; see also FIG. 13). Within these categories, samples from subjects with genetically causal forms of these disorders were also collected (lower part of TABLE 4; see also FIG. 13).

TABLE 4
Data sources for research on MLBD and other disorders with parkinsonism

| | Brain and | | | IPS cell | Longitudinal |
|---|---|---|---|---|---|
| | histopathology | DNA | Fibroblasts | donor line | clinical data |
| Brain, tissue and | | | | | |
| clinical resources | | | | | |
| MLBD, Parkinson's | 157 (refs. | 629 | 42 | 12 | 260 |
| disease or parkinsonism | 41, 88, 89) | | | | |
| Multiple system atrophy | 17 (refs. | 0 | 2 | 2 | 4 |
| | 42, 90) | | | | |
| Progressive | 24 | 0 | 1 | 0 | 6 |
| supranuclear palsy | | | | | |
| Corticobasal | 1 | 0 | 0 | 0 | 0 |
| degeneration | | | | | |
| Fronto-temporal | 1 | 0 | 0 | 0 | |
| dementia | | | | | |
| Azheimer's disease | 8 | 0 | 0 | 0 | 1 |
| without Parkinson's | | | | | |
| disease | | | | | |
| Atypical parkinsonism | 6 | 8 | 2 | 1 | 6 |
| No neurological disease | 23 | 542 | 18 | 9 | Not |
| | | | | | applicable |
| Genetic MLBD or | | | | | |
| parkinsonism | | | | | |
| resources | | | | | |
| Idopathic Parkinson's | 146 | 552 | 14 | 1 | 241 |
| disease | | | | | |
| SNCA any mutation | 2 (refs. | 2 | 1 (ref. | 1 (ref. | 3 |
| | 43, 91, 92) | | 93) | 30) | |
| LRRK2 any mutation | 2 (ref. | 44 (ref. | 15 | 3 (refs. | 8 |
| | 22) | 94) | | 31, 95) | |
| GBA one allele | 4 (ref. | 3 | 2 | 0 | 1 |
| | 96) | | | | |
| GBA both alleles | 0 | 1 | 1 | 1 | 1 |
| LRRK2/GBA compound | 0 | 8 | 4 | 1 | 1 |
| heterozygote | | | | | |
| PARK2 one allele | 2 (ref. | 11 (ref. | 1 | 0 | 0 |
| | 97) | 98) | | | |
| PARK2 both alleles | 1 (ref. | 4 | 2 | 2 | 2 |
| | 98) | | | | |
| PINK1 both alleles | 0 | 2 | 1 | 1 | 1 (ref. |
| | | | | | 99) |
| 22q11 deletion | 0 | 1 | 1 | 1 | 1 |
| No neurological disease | 19 | 542 | 18 | 3 | Not |
| | | | | | applicable |

In additional embodiments, nuclear reprogramming and induced pluripotent stem (iPS) cell technology starting from individual donors with clear clinical diagnoses may be used. This approach could be used to create a disease model from patient-specific human cells (29). In regard to addressing some of the challenges specific to MLBD and parkinsonism, patient-specific iPS cells, in some embodiments, allow one to more clearly understand the differences and/or potential similarities between genetic forms of disease, including allelic variants, and to compare these to idiopathic disease. Establishing a well-defined collection of cell lines taken from patients with thoroughly characterized high-quality clinical data, including data on the peripheral manifestations of the disease (if any) and ideally with confirmative neuropathology at autopsy, is the key to the successful interpretation of findings in some embodiments.

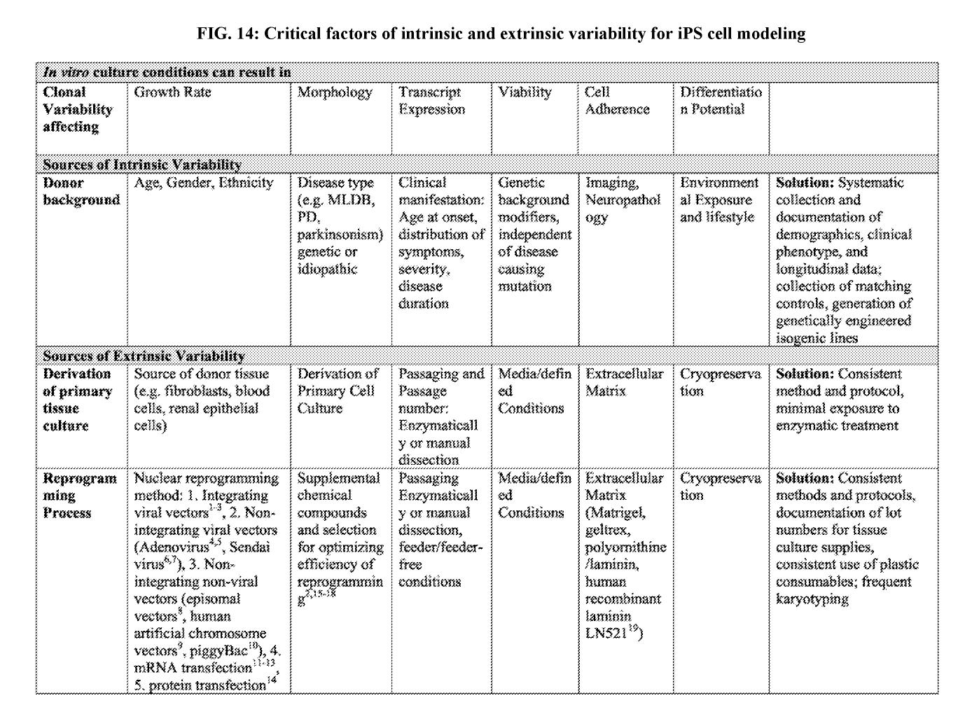

In addition to the need for clinically fully characterized patient donors, several technical challenges must be addressed to reduce variability (TABLE 5 and FIG. 14). Another critical area for improvement is the use of consistent and well-documented methods (see TABLE 5 and FIG. 14).

TABLE 5
Exemplary critical factors of intrinsic and extrinsic variability for iPS cell
modeling.
In vitro culture conditions can result in

| | | | | |
|---|---|---|---|---|
| Clonal | Growth Rate | Morphology | Transcript | Viability |
| Variability | | | Expression | |
| affecting | | | | |

Sources of Intrinsic Variability

| | | | | |
|---|---|---|---|---|
| Donor | Age, Gender, Ethnicity | Disease type | Clinical | Genetic |
| background | | (e.g. MLDB, | manifestation: | background |
| | | PD, | Age at onset, | modifiers, |
| | | parkinsonism) | distribution of | independent |
| | | genetic or | symptoms, | of disease |
| | | idiopathic | severity, | causing |
| | | | disease | mutation |
| | | | duration | |

Sources of Extrinsic Variability

| | | | | |
|---|---|---|---|---|
| Derivation | Source of donor tissue | Derivation of | Passaging and | Media/defined |
| of primary | (e.g. fibroblasts, blood | Primary Cell | Passage | Conditions |
| tissue | cells, renal epithelial | Culture | number: | |
| culture | cells) | | Enzymatically | |
| | | | or manual | |
| | | | dissection | |
| Reprogramming | Nuclear reprogramming | Supplemental | Passaging | Media/defined |
| Process | method: 1. Integrating | chemical | Enzymatically | Conditions |
| | viral vectors¹⁻³, 2. Non- | compounds | or manual | |
| | integrating viral vectors | and selection | dissection, | |
| | (Adenovirus^{4,5}, Sendai | for optimizing | feeder/feeder- | |
| | virus^{6,7}), 3. Non- | efficiency of | free | |
| | integrating non-viral | reprogramming^{2,15-18} | conditions | |
| | vectors (episomal | | | |
| | vectors⁸, human | | | |
| | artificial chromosome | | | |
| | vectors⁹, piggyBac¹⁰), 4. | | | |
| | mRNA transfection¹¹⁻¹³, | | | |
| | 5. protein transfection¹⁴ | | | |
| Neuronal | 1. Spontaneous | Duration, | Passaging | Media/defined |
| Differentiation | differentiation 2. | Plating cell | Manual | Conditions |
| Protocols | Stromal feeder layer co- | density | dissection or | (NSC, |
| | culture (SDIA)^{20,21} 3. | | Enzymatic | DA1, DA2) |
| | Co-culture with | | disruption | |
| | astrocytes 4. Embryoid | | with either | |
| | body differentiation^{22,23}/ | | collagenase | |
| | neurosphere | | vs. Accutase | |
| | generation²⁴, 5. | | | |
| | Differentiation using | | | |
| | small molecule | | | |
| | inhibitors and | | | |
| | recombinant proteins²⁵⁻²⁸ | | | |
| | 6. Use of engineered cell | | | |
| | lines with forced | | | |
| | expression of midbrain | | | |
| | transcription factors²⁹ | | | |

| In vitro culture conditions can result in | | | |
|---|---|---|---|
| Clonal | Cell | Differentiation | |
| Variability | Adherence | Potential | |
| affecting | | | |
| Sources of Intrinsic Variability | | | |
| Donor | Imaging, | Environmental | Solution: Systematic |
| background | Neuropathology | Exposure | collection and |
| | | and lifestyle | documentation of |
| | | | demographics, clinical |
| | | | phenotype, and |
| | | | longitudinal data; |
| | | | collection of matching |
| | | | controls, generation of |
| | | | genetically engineered |
| | | | isogenic lines |
| Sources of Extrinsic Variability | | | |
| Derivation | Extracellular | Cryopreservation | Solution: Consistent |
| of primary | Matrix | | method and protocol, |
| tissue | | | minimal exposure to |
| culture | | | enzymatic treatment |
| Reprogramming | Extracellular | Cryopreservation | Solution: Consistent |
| Process | Matrix | | methods and protocols, |
| | (Matrigel, | | documentation of lot |
| | geltrex, | | numbers for tissue |
| | polyornithine/ | | culture supplies, |
| | laminin, | | consistent use of plastic |
| | human | | consumables; frequent |
| | recombinant | | karyotyping |
| | laminin | | |
| | LN521¹⁹) | | |
| Neuronal | Extracellular | Cryopreservation | Solution: Consistent |
| Differentiation | Matrix | (10% | methods and protocols, |
| Protocols | (MEFs, | FBS/Media, | documentation of lot |
| | matrigel, | Bambanker) | numbers for tissue |
| | geltrex, | | culture supplies, |
| | polyornithine/ | | consistent use of plastic |
| | laminin, | | consumables; unbiased |
| | LN521¹⁹) | | measures of number of |
| | | | dopaminergic neurons, |
| | | | midbrain specificity, |
| | | | neurophysiology |

Multiple different neuronal differentiation protocols have been developed over the last 15 years to differentiate human embryonic stem cells, and now iPS cells, into various types of neurons. It can be challenging to compare neuronal differentiation protocols because of their variable methods and techniques. As a result, studies often do not replicate, and data are difficult to interpret across laboratories. The field is in need of standardized and validated iPS cell laboratory practices that can be used to generate and characterize cells and phenotypes of interest (see TABLE 5 and FIG. 14).

Taking these precautions, and using well-characterized patient material, comprehensive collection of patient-specific skin cell lines and iPS cell clones may be established in some embodiments (see TABLE 4, lower part; see also FIG. 13). There is also evidence that disease is caused by mutations in the SNCA and LRRK2 genes. In additional embodiments, differences between patient-derived cells and control cells derived from unaffected individuals are observed, leading to mitochondrial dysfunction, increased susceptibility to oxidative stress and reduced survival after differentiation (30-40). In some embodiments, by integrating longitudinal clinical assessment and brain neuropathology to establish the diagnoses of MLBD and parkinsonism, this research model reinforces its criteria and securely identifies more of the subtypes.

### Mechanism and Outcomes on Pathology Via Data Resources

In further embodiments, ensuring that information can flow quickly from the laboratory to the clinic and vice versa is critical to collecting the highest-quality diagnostic and clinical data, gathered by trained movement-disorder specialists caring for their patients. In additional embodiments, another component of this model is building a data and tissue bank derived from as many affected individuals as possible and including blood, saliva, DNA, immortalized lymphocytes, skin fibroblasts and libraries of iPS cell lines, as well as ancillary clinical data (for example, imaging studies and data on environmental exposures or nonmotor symptoms of Parkinson's disease). In some embodiments, patients may be asked to sign up for brain donation program (see TABLE 4, upper part; see also FIG. 13). In some embodiments, availability of a repository of well-characterized clinical data and tissue samples in additional embodiments, one could rapidly confirm most individuals with pathologically sporadic Lewy body Parkinson's disease (MLBD) who do not carry the first identified autosomal dominant SNCA variant. (41, 42) One could also confirm the existence of cases caused by SNCA triplication as well as to run the first clinical trial aimed at modifying the course of Parkinson's disease. (41-44) By integrating clinical care and patient participation with over 70 data sources and by anchoring records to neuropathology, one could, in some embodiments, model the diseases and test hypotheses with maximal relevance to clinical delivery and improvement of care. (45) National registries such as the Danish National Patient Register and Swedish patient registries are excellent data sources for calculating the prevalence and incidence of Parkinson's disease. Data such as International Classification of Diseases (ICD) codes for diagnoses are typically collected, along with medication history and hospital treatment codes; however, these registries usually lack more detailed data types such as phenotypic, genetic, environmental and pathological data, and in most cases autopsy data are limited or unavailable. (46, 47)

### Ordination of Disease Subtypes and Associated Genes

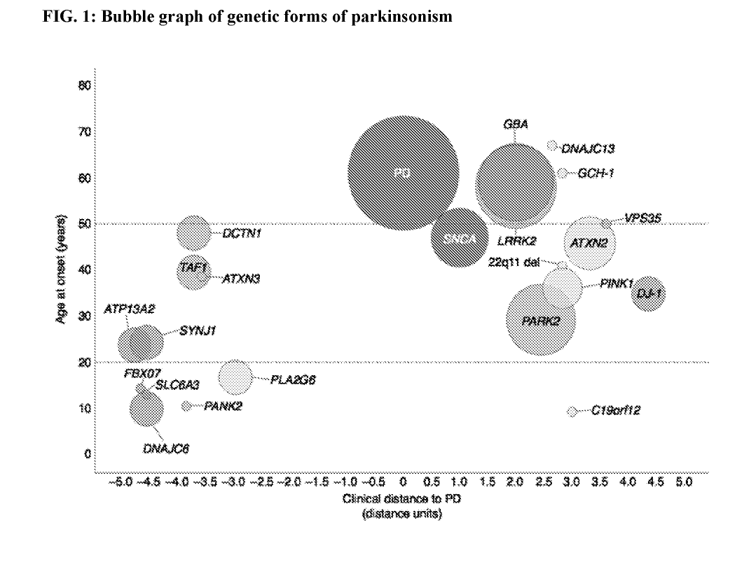

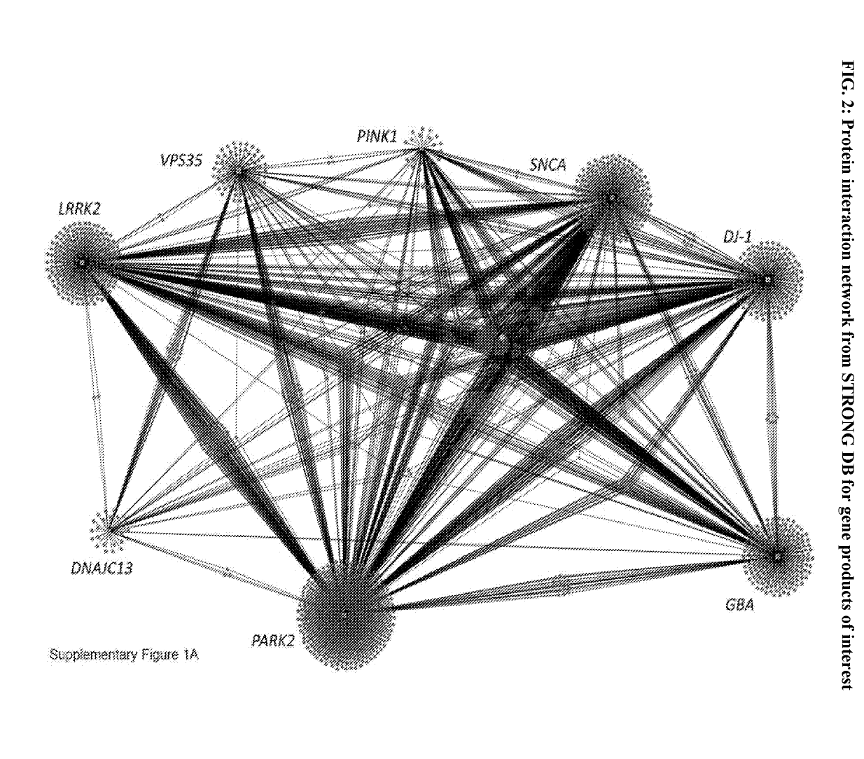

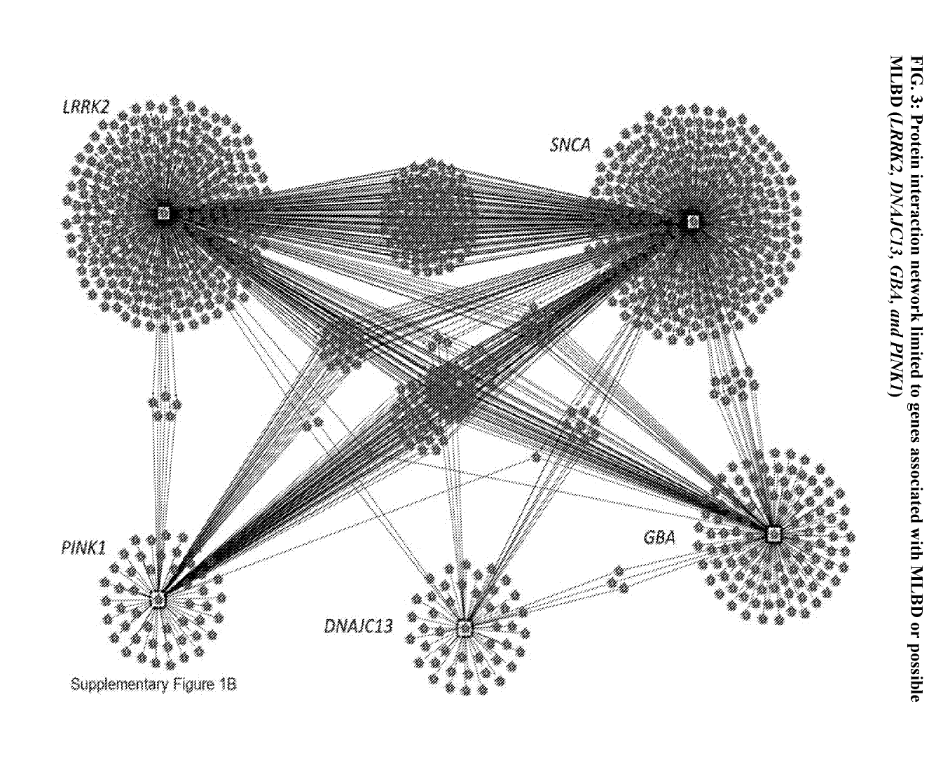

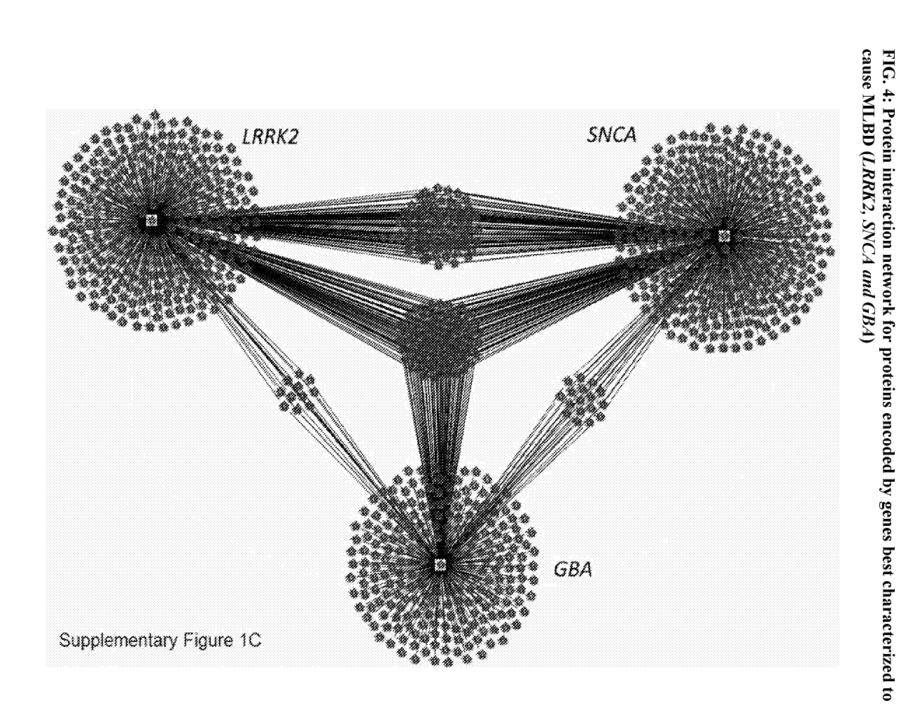

An analytical approach to understanding that there are several mechanistic processes at work in MLBD and other parkinsonian disorders is to examine their clustering in multidimensional space by their quantitative clinical measures. A visual representation of the data from TABLE 1 (see also FIG. 10), which is organized based on consideration of genetics, clinical assessments and neuropathology, is shown in FIG. 1. Using the 21 clinical symptoms and 8 neuropathologic categories to calculate the Euclidean distance from Parkinson's disease (see TABLE 6 and FIG. 15). With the visual representation of clinical features by Euclidean distance, age distribution and robustness of evidence for pure Lewy body pathology, it becomes clear that mutations in these genes can cause very different clinical and neuropathological phenotypes in some embodiments.

Another way to consider using a mechanistic approach is to focus searches for interacting proteins by pathological classification (see FIG. 2). In some embodiments, querying the protein interaction networks of gene-related protein products associated with parkinsonism, using their pathological substrate as a guide, could yield important physiological interactions or sets of related genes based on common pathways whose disruption could result in Parkinson's disease. Although the majority of data queried are based on cell type—specific interactions of wild-type proteins, these might be relevant to disease processes in cells expressing the products of mutated genes through disruption of these normal physiologic interactions.

In additional embodiments, MLBD forms a core of sporadic cases of Parkinson's disease and includes at least three genetic subtypes based upon summaries of allelic heterogeneity, clustering of multiple quantitative clinical features and the protein-protein interactions attributable to the products of genes associated with MLBD and other parkinsonian disorders. First, detailed examination of 25 years of patient data and samples, in combination with a comprehensive literature review, suggests a unified entity of primary Lewy body diseases, which is preferably referred to as ‘multisystem Lewy body disease’ in some embodiments, including Parkinson's disease, DLB and PAF. Second, in additional embodiments, on the basis of the clinical, neuropathological and peripheral autonomic features of all forms of parkinsonism associated with genetic causes, only three genes fall into the category of MLBD-associated genes: SNCA, LRRK2 and GBA (TABLE 1 and FIG. 10 and FIG. 1). Third, in some embodiments, the proteins encoded by these three genes showed the highest number of first-degree interactors in protein-protein network (FIG. 4). Fourth, in additional embodiments, these overlapping genes are the ones most consistently reported in GWAS studies associated with clinically characterized Parkinson's disease (5, 19) (a pattern that is not consistently found in other neurodegenerative disease, such as Alzheimer's disease (50)). These approaches are largely independent in Nature and exploratory (clinical and pathological observation, protein-protein interaction and genome-wide association), yet they yield the similar finding, adding support to an unified hypothesis of multisystem Lewy body disease in some embodiments. Some embodiments of the claimed invention can stimulate data-driven discussion of similarities and differences in the several mechanisms operating in parkinsonian movement disorders that will change the emphasis of the field to encourage the use a common language and allow this research move forward with greater clarity and speed.

The present disclosure relates to a novel approach for classifying or parsing out complex diseases or conditions with a wide range of etiologies into subclasses or subtypes based on a common biological pathway or mechanism of associated genes. One embodiment of such approach involves collecting and analyzing a combination of factors (e.g., two or more, three of more, four or more, or five factors) from patients, including clinical symptoms, motor or non-motor symptoms, neuropathology, formation of Lewy bodies, gene, genetic mutation, family history, age at onset, and symptoms involving the peripheral autonomic system, such as the cardiovascular and the enteric nervous system. Motor symptoms include one or more of muscle rigidity, tremor, gait and postural abnormalities, a slowing of physical movement (bradykinesia) and, in extreme cases, a loss of physical movement (akinesia). Non-motor symptoms include, for example, cardiac scan or measurements of the gastrointestinal (GI) motility. Neuropathology includes formation of Lewy bodies in nerve cells or a sample from a patient. Each of the factors can be assigned a quantitative score, such as 1-5, indicative of the frequency observed among patients in a given population analyzed. The prevalence of the factors and the frequency of genetic forms of all patients analyzed can be plotted to visualize how the factors are distributed across a given patient population. In some embodiments, a distance matrix or other means of capturing all the information from the measured factors or variants in a given patient population can be used to assess how the factors are distributed across the patient population and to determine if genetic forms cluster together. For genetic forms that cluster together, protein interaction networks can be generated for each of the genes in the cluster to determine overlap in their function and/or protein-protein interactions, wherein significant overlap in protein-protein interactions or protein interaction networks is indicative of a common biological pathway or mechanism. Genes that cluster can then be used to redefine a subclass of a disease or condition. Clustering of any of the other factors can also be used to redefine subclasses of a diseases or condition.

In one aspect, disclosed herein is a method of distinguishing a disease from multiple diseases associated with similar symptoms comprising (a) building a tissue bank derived from samples of a plurality of subjects displaying at least one symptom of the similar symptoms; (b) characterizing each of the samples by performing at least one of sequencing a nucleic acid, quantifying a nucleic acid or a protein, detecting a histopathological abnormality, and detecting a protein-protein interaction; (c) building a data bank derived from assessing the subjects, wherein the data comprises information selected from at least one of: the at least one symptom, age of disease onset, and environmental circumstances of the subjects; (d) identifying a sub-group in the plurality of subjects, wherein the sub-group possesses at least one similar tissue characteristic and at least one data characteristic; and (e) determining whether the sub-group has the disease. The disease may be a neurological disease or condition, a neurodegenerative disease or condition, a neuromuscular disease or condition, a liver disease or condition, a gastrointestinal disease or condition, a metabolic disease or condition, or an autoimmune disease or condition. The plurality of subjects may comprise at least ten subjects, at least fifty subjects, or at least a hundred subjects. The sequencing may comprise sequencing at least a portion of a gene or gene transcript known to harbor a genetic mutation. The genetic mutation may be associated with at least one disease of the multiple diseases. The portion of the gene may be at least about ten nucleotides. The building the tissue bank may comprise freezing the samples, and the samples may comprise a fluid sample selected from a blood sample, a saliva sample, a urine sample, a spinal fluid sample, a plasma sample, or a lymphatic fluid sample. In addition, the samples may comprise tissue samples, biopsy samples, cadaver samples, or while cells. The quantifying the nucleic acid may comprise quantitative PCR. The detecting the histopathological abnormality may comprise contacting the sample with a stain or a detectable tag-conjugated antibody. The building the data bank may comprise administering a questionnaire to the subjects. In some embodiments of the method, at least one of the proteins involved in the protein-protein interaction are known to be involved in a biological pathway implicated in any one of the multiple diseases.

Also disclosed herein is a method of distinguishing a first disease from a second disease, wherein the first disease and the second disease are associated with similar symptoms comprising (a) collecting biological samples from a plurality of subjects displaying at least one symptom of the similar symptoms; (b) sequencing a nucleic acid in the biological samples to identify a subgroup of the plurality of subjects expressing a genetic mutation; (c) recording at least one symptom experienced by the plurality of subjects; (d) identifying a sub-group in the plurality of subjects, wherein the sub-group possesses the genetic mutation and displays the at least one symptom; and (e) determining the sub-group has the disease. The method may further comprise assessing a test subject for having the disease comprising (a) collecting a biological sample from the test subject; (b) sequencing or quantifying a nucleic acid or a peptide in the biological sample; (c) observing at least one symptom experienced by the test subject; and (d) the subject as having the disease when the subject possesses the genetic mutation and displays the at least one symptom. In addition, the method may comprise treating the test subject with an agent specific for the disease.

In one embodiment, a method of defining or parsing out complex diseases or conditions with a wide range of etiologies into subclasses or subtypes based on a common biological pathway or mechanism of associated genes, involving the steps of collecting patient data, analyzing two or more factors of the following factors to determine an observed frequency of each factor in a given patient population, including data on family history, genetic mutation, motor symptom, non-motor symptom, neuropathology, age of onset, and symptoms involving peripheral autonomic system; mapping observed frequencies of the various factors to determine a cluster of the analyzed factors, linking the cluster of analyzed factors to one or more genes to determine the genes underlying a subclass corresponding to the observed frequencies of the analyzed factors; analyzing protein-protein interactions of the genes linked to the subclass to validate a common biological pathway or mechanism; and defining the subclass as a distinct disease or condition based on the underlying mechanism identified. In some cases, the complex diseases or conditions comprise Parkinson's disease and parkinsonian diseases or conditions. In other cases, the complex disease or conditions comprise dementia, Alzheimer's disease, or a cancer. Such method can be applied to identify the subclass of multisystem Lewy body disease (MLBD). As disclosed herein, peripheral autonomic system involves assessing the gastrointestinal (GI) system for dysfunction and/or cardiac abnormality. In some embodiment, motor symptoms include one or more of muscle rigidity, tremor, gait and postural abnormalities, a slowing of physical movement (bradykinesia), and a loss of physical movement (akinesia), while non-motor symptoms comprise symptoms measurable by a cardiac scan or symptoms relating to gastrointestinal (GI) motility. In some cases, neuropathology comprises formation of Lewy bodies in a sample of nerve cells extracted from a subject. In some embodiments, observed frequencies or prevalence of analyzed factors in a given patient population can involve mapping frequencies using distance matrices or plotting out Euclidean distances to visualize clustering of certain factors, such as genes. Gene mutations involved in MLBD or parkinsonian diseases can include one or more mutations in LRRK2, GBA, SNCA, VPS35, DJ-1, PINK1, PARK2, DNAJ13C, and any combination thereof. In some embodiments, three genes are predominantly associated with MLBD or Parkinson's disease, such as LRRK2, GBA, SNCA, and any combination thereof.

Also disclosed herein is a method of characterizing a complex disease or condition comprising: identifying one or more allelic variants in one or more genes associated with the disease or condition; determining clinical pathology or symptoms associated with each allelic variant in a patient population; grouping the genes with allelic variants based on the degree of overlap between their clinical pathology or symptoms and a standard set of clinical pathology or symptoms; determining proteins and/or genes that interact with each group of genes with allelic variants to construct protein interaction networks that inform the molecular mechanism or cellular process affected by the allelic variants; and characterizing said disease or condition based on the molecular mechanism or cellular process associated with one or more allelic variants. In such cases, the complex disease or condition can be multisystem Lewy body disease, Parkinson's disease, or Parkinsonism; wherein one or more allelic variants is selected from the group consisting of: LRRK2, GBA, SNCA, VPS35, DJ-1, PINK1, PARK2, DNAJ13C, and any combination thereof; and wherein the standard set of clinical pathology or symptoms refers to Parkinson's disease. In some cases, group of genes used to construct protein interaction networks for understanding the underlying pathway or mechanism include any one of the following groups: LRRK2, GBA, and SNCA; LRRK2 and SNCA; LRRK2 and GBA; or GBA and SNCA.

In some cases, a method of treating a disease or condition involves diagnosing a subject, which can be a human or a mammalian, using any of the methods above. In some instances, the subject is diagnosed with MLBD. In some cases, the method involves administering one or more of the following therapeutic agents to the subject: L-dopa, monoamine oxidase B inhibitor, dopamine agonist, catechol-O-methyltransferase inhibitor, anticholinergic, amantadine, or any combination thereof.

Another method disclosed herein involves treating a disease or condition, which can be MLBD, Parkinson's disease, or parkinsonian, comprising the steps of: obtaining a genetic sample from a patient; sequencing the genetic sample for one or more genes associated with the disease or condition; identifying one or more allelic variants in the genes associated with the disease or condition; identifying proteins and/or genes that interact with the genes associated with the disease or condition to determine the molecular mechanism or cellular process affected by the allelic variants; and administering a therapy or pharmaceutical agent directed to the molecular mechanism or cellular process affected by the allelic variants. In some cases the allelic variant is a gene selected from the group consisting of: LRRK2, GBA, SNCA, and any combination thereof. In other cases, one or more allelic variants are in: LRRK2, GBA, and SNCA; LRRK2 and SNCA; LRRK2 and GBA; or GBA and SNCA. In some embodiments, the therapy or pharmaceutical agent includes L-dopa, monoamine oxidase B inhibitor, dopamine agonist, catechol-O-methyltransferase inhibitor, anticholinergic, amantadine, or any combination thereof.

Also disclosed herein is a method of distinguishing a first disease from a second disease, wherein the first disease and the second disease are associated with similar symptoms comprising (a) collecting biological samples from a plurality of subjects displaying at least one symptom of the similar symptoms; (b) quantifying a nucleic acid in the biological samples to identify a subgroup of the plurality of subjects expressing an abnormal amount of the nucleic acid relative to an average amount of the nucleic acid expressed by a healthy population; (c) recording at least one symptom experienced by the plurality of subjects; (d) identifying a sub-group in the plurality of subjects, wherein the sub-group expresses the abnormal amount and displays the at least one symptom; and (e) determining the sub-group has the disease. The method may comprise assessing a test subject for having the disease comprising (a) collecting a biological sample from the test subject; (b) sequencing or quantifying a nucleic acid or a peptide in the biological sample; (c) observing at least one symptom experienced by the test subject; and (d) identifying the subject as having the disease when the subject expresses the abnormal amount and displays the at least one symptom. Further, the method may comprise treating the test subject with an agent specific for the disease.

Application of this systematic approach as described herein led to the identification of MLBD as a new subclass, which includes Parkinson's disease that is strongly associated with three genes: LRRK2, SNCA, and GBA.

In some embodiments, methods of diagnosis and treatment of MLBD, including Parkinson's disease, involves performing an assessment of two or more the factors described herein, such as motor symptoms, non-motor symptoms, mutation in one or more of LRRK2, SNCA, and GBA, neuropathology, and symptoms of the peripheral autonomic system, including the heart using cardiac MIBG scintigraphy scan and the enteric nervous system using GI motility measurements. Based on observed frequencies or prevalence of each of the assessed factors in the MLBD patient population, a new patient is given a quantitative score for each of the assessed factors to determine where the patient maps relative the analyzed patient population in a distance matrix or a plot. In other words, a patient's data is compared against observed frequencies of the factors in MLBD or Parkinson's patients to determine the likelihood of having or developing the same subclass of the disease or condition relative to observations in the patient population. A quantitative score can be used to indicate a patient's score or map of assessed factors assessed as compared to a given patient population, such as a score of 1-5, wherein a high score refers to a positive diagnosis of the disease or condition, while a low score indicates a low chance of having or developing the disease or condition. Upon diagnosis, a patient can be treated with drugs that target the underlying genes known to associate or cluster with the patient's disease subclass.

For Parkinson's disease, drugs that target deficiencies in LRRK2, SNCA, and/or GBA can be used to treat a patient with a positive diagnosis for PD.

The SNCA gene encodes alpha-synuclein, which is involved in MLBD, including PD. Treatments to control symptoms of PD include, but not limited to, dopamine promoters to stimulate dopamine receptors (e.g. bromocriptine and amantadine) in the brain, antidepressants (selegiline and rasagiline) to prevent/relieve depression, cognition-enhancing medications (e.g. rivastigmine) to improve mental function and lower blood pressure, anti-tremor medication (e.g. benztropine), and physical exercise. For deficiencies in LRRK2, LRRK2 modulators (e.g., LRRK2 inhibitors) and Hsp90 inhibitors can be used in MLBD and PD patients. Mutations in the GBA gene are linked to PD and Gaucher disease. For Gaucher disease, the treatment is glucocerebrosidase enzyme replacement therapy. In some cases, glucocerebrosidase enzyme replacement therapy may also work to treat MLBD or PD or alleviate symptoms of MLBD or PD. In some cases, glucocerebrosidase enzyme replacement therapy comprises one or more chaperones for enhancing glucocerebrosidase enzyme crossing the blood brain barrier.

Treatments for PD can be used to treat patients with MLBD, including, but not limited to, carbidopa-levodopa, wherein levodopa, is converted to dopamine in the brain. Levodopa can be combined with carbidopa (Rytary, Sinemet), which protects levodopa from premature conversion to dopamine before reaching the brain. Other MLBD treatments include dopamine agonists, MAO-B inhibitors, such as selegiline (Eldepryl, Zelapar) and rasagiline (Azilect), which prevent the breakdown of brain dopamine by inhibiting brain enzyme monoamine oxidase B (MAO-B). Catechol-O-methyltransferase (COMT) inhibitors, such as Entacapone (Comtan), prolong the effects of levodopa therapy by blocking an enzyme that breaks down dopamine. Anticholinergics help to control some of the motor symptoms, such as tremor, associated with PD, and include, but not limited to, benztropine (Cogentin) and trihexyphenidyl. Amantadine can also be used to provide short-term relief of symptoms. Other therapies include deep brain stimulation, gene therapy, and administration of antibodies or immunotherapies that help to reduce or degrade alpha-synuclein or aggregates thereof.

Patients with MLBD or PD often suffer GI dysfunction. The signs and symptoms of GI dysfunction observed may include dysphagia, gastroparesis, prolonged GI transit time, constipation, and difficulty with defecation. There are currently no good methods to assess disease burden or to measure the benefit of treating the GI symptoms. Ability to treat GI symptoms properly can significantly impact the efficacy of drugs used to treat or manage Parkinson's disease and symptoms. As such, there is a need for better diagnosis and treatment of GI conditions, especially in patients who are at risk for Parkinson's disease. Additionally, as described herein in various embodiments, the enteric nervous system can serve as a proxy or a model for the central nervous system, which provides an in vivo model for developing and testing therapeutics and diagnostics for MLBD, Parkinson's disease, other neurodegenerative diseases, as well as GI conditions. In some embodiments, GI cells can be biopsied and studied ex vivo to identify or screen neuroprotective agents or therapeutics, or to undergo gene therapy to correct or repair a gene related to a MLBD or PD before returning the modified cells to a subject. The combination of quantitative and qualitative analysis of the enteric nervous system can provide a novel approach for early diagnosis and treatment of Parkinson's disease or other MLBDs before irreparable nerve damage occurs in the central nervous system (CNS).

### MLBD and the Enteric Nervous System

A major challenge in MLBD and PD is identifying patients early in the disease process in order to ensure that patients are seen by qualified professionals as early as possible, and to administer disease-altering interventions. Patients with MLBD or PD may manifest the disease first in the GI tract. GI dysfunction may precede the onset of motor symptoms in MLBD and PD patients by decades. Since such patients may not have the classic motor symptoms, they often go years with poorly treated GI symptoms and other early signs of Parkinson's disease, and are not referred to a specialist until they have progressed substantially.

Early treatment of patients with MLBD and PD with the monoamine oxidase (MAO) inhibitors, selegiline and rasagiline, may slow disease progression. Importantly, an intensive exercise program started early in the disease process can delay the need for treatment with L-dopa. As such, there is a need to identify these patients early and provide every opportunity for optimal therapeutic interventions.

Despite treatment with rasagiline and high intensity exercise, MLBD and PD may continue to progress in some patients. In spite of years of efforts to find drugs and interventions that prevent the progression of the disease, there are no new therapies on the horizon. There is a need for good animal models that are predictive of clinical efficacy of therapies.

The neurodegenerative process occurring in the brains of patients with Parkinson's disease may also occur in the enteric nervous system. Thus, it is also plausible that the neurons of the GI system are some of the first to be affected, even prior to the disease process in the brain. If this is the case, then the GI system and manifestations of GI symptoms may be useful to study the overall disease process. Since the enteric nervous system in humans and rodents are rather similar, rodents may manifest disease in the GI tract in a manner that closely models the human condition.

The peripheral autonomic nervous system plays an important role. Alpha-synuclein-positive Lewy bodies and Lewy neurites have been identified postmortem in a wide variety of areas of the body, ranging from the myenteric plexus of the gut to the salivary gland, in patients diagnosed with Parkinson's disease. In particular, loss of dopaminergic neurons of the substantia nigra pars compacta (SNpc) and their nigrostriatal projections produce parkinsonism, the movement disorder characterized by tremor, bradykinesia, rigidity, and postural instability that are the most obvious clinical signs of PD. Importantly, α-synuclein aggregates are found in the SNpc and are thought to directly correlate or potentially be the cause of dopaminergic neuronal cell loss in the brain. These abnormal accumulations of α-synuclein (aggregates) are referred to as Lewy bodies, the neuropathological hallmark of PD. However, it is well understood that PD affects more than just the central nervous systems (CNS), giving rise to multiple other non-motor symptoms. Importantly, increasing evidence has now linked pathological accumulation of α-synuclein to neuronal loss in the enteric nervous system in PD. These Lewy bodies are assumed to be the cause of the GI tract symptoms.

GI dysfunction in MLBD and/or PD can occur early in disease progression. Causes of GI dysfuncation can include damage to the enteric nervous system (ENS). Studies of the GI tract in PD can offer an opportunity to understand the disease better and to detect it earlier-potentially before the neurons in the brain are attacked. The ability to identify early stage PD would allow clinicians to begin interventions before dopaminergic neurons start to die. Neurodegenerative process occurring in the brain of a patient with MLBD and/or PD can occur in the ENS of the patient. In some cases, neurons of the GI/enteric nervous system are among the first affected by MLBD and/or PD pathology, even prior to the damaging process in the brain (“premotor” stage).

Neurodegenerative process occurring in the brain of a patient with PD can also occur in the ENS. These neurons of the GI system can be some of the first to be affected by PD pathology, even prior to the damaging process in the brain (“premotor”). The GI system and manifestations of GI symptoms can be used to study the overall PD process. Through the study of neuron loss in the ENS, new neuroprotection strategies and objective assessments can be identified. Development of medications that block α-synuclein accumulation in the ENS of the GI tract may also prevent α-synuclein accumulation in the brain, and thus provide treatment options for “premotor” and motor symptoms.

MLBD or PD may result in progressive accumulation of α-synuclein in the neurons of the GI tract, resulting in dysfunction and ultimately degeneration of neurons in the GI tract. This may lead to clinical symptoms that can be quantified and measured over time. Quantitative assessments using esophageal and anorectal manometry, the SmartPill, and G-Tech monitoring device may be more sensitive at quantifying GI symptoms and may be better endpoints for clinical studies designed for drug approvals than the validated GSRS and GCSI scales. A physician and patient designed survey focused on Parkinson's disease-specific GI symptoms, delivered by email between visits, may be a better predictor of clinically meaningful changes in progression or improvement than currently available instruments. Analysis of medication usage and correlation to outcomes may help determine if there is a benefit to Parkinson's specific therapies. Analysis of medication usage and correlation to outcomes may help determine if there is a benefit to GI specific therapies.

Neuron loss in the enteric nervous system prior to the manifestation of motor symptoms in patients with MLBD or PD can be a predictor of disease progression. Novel neuroprotectant strategies and objective assessments can be identified by studying the ability to prevent or impede neuronal loss in the enteric nervous system. For example, the development of medications that block α-synuclein accumulation in the GI tract may also prevent α-synuclein accumulation in the brain, and thus provide treatment options for the motor symptoms of Parkinson's disease.

The present disclosure includes methods for assessing and treating GI symptoms in subjects suffering neurological conditions (e.g. Parkinson's disease) based on a characterization of GI properties using acceptable quantitative metrics and scales, such as the SmartPill, G-Tech device, and manometry, as well as the GI Symptom Relief Scale (GSRS) and Gastroparesis Cardinal Symptom Index (GCSI). In addition expression and accumulation of genes/proteins (e.g. α-synuclein) can be assessed in the GI tract.

In one aspect, the present disclosure provides methods, processes and systems for the diagnosis and/or treatment of MLBD and/or PD using quantitative GI tract measurements as diagnostic tools and/or as biomarkers. In some embodiments, GI measurements can be used to identify the natural history of GI dysfunction in MLBD and/or PD. In some embodiments, GI measurements can be used to identify the key premotor markers of MLBD and/or PD.

In some embodiments, the methods comprise using MLBD and/or PD-specific GI “markers” to diagnose MLBD and/or PD. In some embodiments, the methods comprise using MLBD and/or PD-specific GI “markers” to treat symptoms of MLBD and/or PD. In some embodiments, the methods comprise using MLBD and/or PD-specific GI “markers” to develop treatments that alter the pathogenesis of MLBD and/or PD

In some embodiments, the GI-specific processes, measurements, tools and/or markers include high resolution esophageal manometry (HRM), high resolution anorectal manometry (HRAM), wireless motility capsules, GI symptom questionnaires, or any combination thereof. In some embodiments, the questionnaires are validated, e.g. have been used to achieve regulatory approval for a new therapy.

The human enteric nervous system (ENS) contains approximately 500 million neurons and 4 times as many glia distributed along the entire bowel in two interconnected layers called the submucosal and myenteric plexus. These neurons and glia control bowel motility, respond to sensory stimuli, regulate blood flow, support epithelial function and modulate local immunity. To perform these roles, there are at least 14 enteric neuron subtypes that express every neurotransmitter in the CNS and several types of enteric glia.

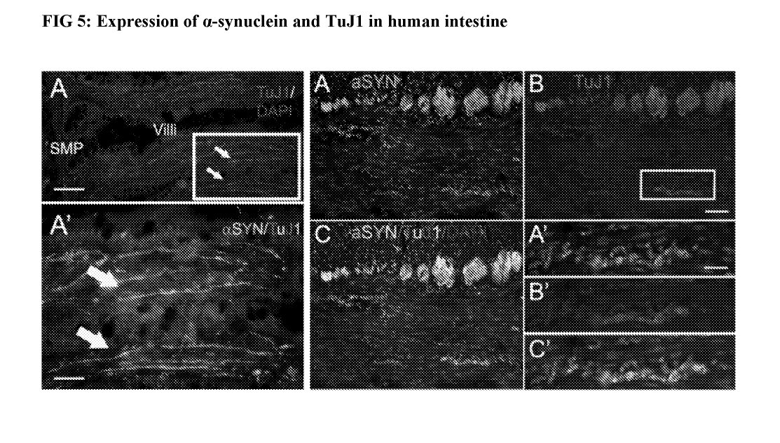

FIG. 5 provides exemplary immunohistochemical images that confirm the presence of α-synuclein throughout the ENS in human tissue. Alpha-synuclein (green), TuJ1 (neurons—red) and nuclei (blue) demonstrating α-synuclein is present in ENS neurons (merge=yellow). Panel A-A′ shows TuJ1 stained long sensory axons in the submucosal plexus (SMP) (Bar=25 μm). Panel A′ is a high magnification of inset that reveals co-label of α-synuclein in axons stained by TuJ1. Bar=10 μm. Panel A-C shows α-synuclein (A) and TuJ1 (B) in the neural network of the outer longitudinal muscle wall and co-label (C) (Bar=100 μm). Panel A′-C′ is a high magnification of inset in B show co-label (C′ yellow) with α-synuclein (A′) and TuJ1 (B′) (Bar=10 μm).

### Methods of Assessing GI Symptoms

Disclosed herein are methods of screening a subject for a neurological condition, comprising: performing an assessment of a GI condition; assigning a quantitative value to the GI condition based on the assessment; comparing said quantitative value to a value range predetermined to be indicative of the neurological condition; and identifying said subject as suffering from or prone to the neurological condition if said quantitative value falls in said value range. A GI condition may be selected from a GI symptom, a GI function, a GI rate, a GI gene/protein expression, a GI measurement, and combinations thereof. The neurological condition may be selected from Parkinson's disease and Parkinson's-like disease.

Further disclosed herein are methods of screening a therapy for therapeutic efficacy towards a neurological condition and/or symptoms thereof, comprising: performing an assessment of a GI condition; assigning a quantitative value to the GI condition based on the assessment; comparing said quantitative value to a value range predetermined to be indicative of the neurological condition; and identifying said subject as suffering from or prone to the neurological condition if said quantitative value falls in said value range. A GI condition may be selected from a GI symptom, a GI function, a GI rate, a GI gene/protein expression, a GI measurement, and combinations thereof. The neurological condition may be selected from Parkinson's disease and Parkinson's-like disease.

The methods may include recording the dates of diagnosis and symptom(s) onset. The date of Parkinson's diagnosis may be recorded, as well as Parkinson's symptoms experienced by the patient since the time of diagnosis. Current Parkinson's symptoms may also be recorded. The onset date of GI symptoms may be recorded, as well as all GI symptoms experienced by the patient since the time of diagnosis of the GI symptoms. The methods may further comprise recording existing medication requirements and family history of Parkinson's disease,

The following scales and assessments may be used to evaluate the progression of GI symptoms in patients with Parkinson's disease or patients at risk for developing the motor symptoms of Parkinson's disease:

### Esophageal Manometry

The methods disclosed herein may comprise performing esophageal manometry. Esophageal manometry may be used to evaluate the functioning of the esophageal sphincters. Esophageal manometry may be used to evaluate the functioning of the upper and lower esophageal sphincters and motility. Esophageal manometry may be used to evaluate the tone and the motility of the sphincters. This procedure may be performed with or without sedation and lasts ˜15 min. The method may comprise inserting a thin catheter through the locally anesthetized nose. The catheter may incorporate an assembly for the measurement of pressure and bidirectional fluid movement (impedance) during several water swallows.

### Anorectal Manometry

The methods disclosed herein may comprise performing anorectal manometry. Anorectal manometry may be used to evaluate anorectal motility. Anorectal manometry may include the following: 1) anal sphincter function, 2) rectoanal reflex activity, 3) rectal sensation, 4) changes in anal and rectal pressures during attempted defecation, 5) rectal compliance, and 6) performance of a balloon expulsion test. This procedure may be performed with or without sedation and may last ˜15 min. The Investigator inserts a narrow, flexible tube into the anus and rectum. Once the tube is in place, a small balloon at the tip of the tube may be expanded and the patient is asked to squeeze and relax the anus.

### SmartPill®

The methods disclosed herein may comprise use of a SmartPill®. ^{The} SmartPill® may be used to measure gastric, colon, and small bowel transit times. The SmartPill® is an ingestible capsule used to evaluate motility disorders throughout the GI tract and may be used to measure pH, pressure, and temperature over time through the GI tract. Data may be transmitted to a receiver worn by the patient until the SmartPill is expelled. Data from the patient's receiver may the be downloaded at the clinic. The SmartPill may measure the movement of material through the GI tract, as well as the acidity, pressure, and temperature of the stomach and small and large intestines. These measurements may be used to determine transit time through the GI tract. The SmartPill may send information by radio wave signals to a receiver, which stores the signals on a computer chip. The SmartPill procedure may be performed in accordance with the procedure outlined in Patient medications may be withheld during SmartPill® use.

### G-Tech Monitoring Device

The methods disclosed herein may comprise use of a G-Tech monitoring device or a similar device. The G-Tech monitoring device may evaluate the GI myoelectric activity from the surface of the abdomen. The G-Tech monitoring device may be considered to be similar to an ECG for the GI system. The device may be worn for about 4 hours on the abdomen. The G-Tech device may produce myoelectric activity data. The data may be downloaded at a clinic. The myoelectric activity data may be examined for peaks corresponding to the stomach, small intestine, and colon. The G-Tech device will be attached to the patient's abdomen.

### GI Symptom Relief Scale (GSRS)

The methods disclosed herein may comprise use of a GI Symptom Relief Scale (GSRS). The GSRS, or modifications thereof, may be used in this study to quantify patients' GI symptoms. The GSRS may consist of about 15 questions, each answered on a 4-point scale, for a total score ranging from 0 (no GI symptoms) to 60 (worst GI symptoms). Questions may be based on the following 5 domains: abdominal pain, reflux, indigestion, diarrhea, and constipation and may be asked with a 2-week recall period. The GSRS may be completed by the Investigator via patient interview.

### Gastroparesis Cardinal Symptom Index (GCSI)

The methods disclosed herein may comprise use of a Gastroparesis Cardinal Symptom Index (GCSI). The GCSI may consists of 3 subscales completed by patients that measure important symptoms related to gastroparesis: nausea/vomiting (3 items), post-prandial fullness/early satiety (4 items), and bloating (2 items). Responses range from 0 (none) to 5 (very severe), with a 2-week recall period. Since patient's with Parkinson's disease often experience delayed gastric emptying or gastroparesis (Kuo, et al., 2010), this scale may be used to determine the symptomatic levels of gastroparesis seen in these patients.

### Hoehn and Yahr Scale

The methods may further comprise use of a Hoehn and Yahr Scale or Modified Hoehn and Yahr Scale. The Hoehn and Yahr Scale defines the staging for broad categories of motor function in patients with Parkinson's disease, start date of GI symptoms, current GI symptoms, physical examination, neurological examination, dietary changes, and vital signs.

The Hoehn and Yahr staging may be used in diagnosis of primary symptoms in a subject. The Hoehn and Yahr scale is a commonly used system for describing how the symptoms of Parkinson's disease progress (Hoehn M, Yahr M (1967). “Parkinsonism: onset, progression and mortality” Neurology 17 (5): 427-42). The scale allocates stages from 0 to 5 to indicate the relative level of disability.

Stage 1: Symptoms on one side of the body only.

Stage 2: Symptoms on both sides of the body. No impairment of balance.

Stage 3: Balance impairment. Mild to moderate disease. Physically independent.

Stage 4: Severe disability, but still able to walk or stand unassisted.

Stage 5: Wheelchair-bound or bedridden unless assisted.

### University of Pennsylvania Smell Identification Test (UPSIT)

The methods disclosed herein may further comprise analyzing non-motor features of Parkinson's disease or Parkinson's-like diseases, including testing of changes in sense of smell and evaluation for other features such as autonomic dysfunction, and changes in mood and cognition.

The methods disclosed herein may comprise use of a University of Pennsylvania Smell Identification Test (UPSIT). The UPSIT is a patient-administered tool used to measure a patient's ability to detect odors. It is known that olfactory bulb volumes decrease in Parkinson's disease and olfactory deficits are seen in many patients. The UPSIT contains 40 odors that the patient will “scratch and sniff” and attempt to identify. Responses are recorded as correct identification of smell or incorrect identification of smell. The total score ranges from 0 (worst) to 40 (best). The UPSIT is a series of four 10 page booklets with the scratch/sniff pad and 4 choices for the scent origin on each page. Patients will be instructed to mark the scent or origin. Study personnel will place to score on the back page of each booklet to indicate the total number of correct responses noted by the patient.

### Electrocardiograms (EKGs)

The methods disclosed herein may comprise use of an electrocardiogram. A 4-lead EKG rhythm strip may be obtained with the patient in a supine position following at least a minute rest. The rhythm strip may record about 1 minute, about 2 minutes, about 3 minutes, about 4 minutes, about 5 minutes, about 6 minutes, about 7 minutes, about 8 minutes, about 9 minutes, about 10 minutes, about 15 minutes, about 20 minutes, about 25 minutes, about 30 minutes, about 45 minutes or about 60 minutes of EKG rhythm.

The methods may comprise a producing an EKG. An EKG is used as a simple, non-invasive, and low-cost screening tool for pre-motor/prodromal Parkinson's disease or Parkinson's-like disease that can be incorporated into routine physical examinations of individuals.

The EKG may display a heart rate variability (HRV) result. The HRV result may comprise a time domain measure. The measure may be selected from the group consisting of standard deviation of R-R intervals (SDNN), the standard deviation of the heart rate (SDHR), the root mean square difference of successive RR intervals (RMSSD), and the percentage number of consecutive RR intervals differing by more than 50 msec (pNN50). The EKG may be obtained for about 1 minute, about 2 minutes, about 3 minutes, about 5 minutes, about 6 minutes, about 7 minutes, about 8 minutes, about 9 minutes, about 10 minutes, about 15 minutes, about 20 minutes, about 3-5 minutes, about 5-10 minutes, or about 15-20 minutes.

The HRV may comprise a geometric/non-linear measure. These include measures derived from the Poincaré plot, which is a graphical representation of the relationship between consecutive RR intervals, where an RR interval is plotted against the preceding RR interval. The short term HRV (beat-to-beat) is calculated perpendicular to the line of identity (SD1) and the long term (overall HRV) is calculated along the line of identity (SD2). Geometric measures include RR triangular index and the triangular interpolation of NN (TINN).

In some embodiments, the HRV result comprises a frequency domain measure, e.g., Very Low Frequency (VLF) (0-0.04 Hz), Low Frequency (LF) (0.04-0.15 Hz), or High Frequency (HF) (0.15-0.4 Hz). The values can be reported in both absolute values and normalized units. In some embodiments, the frequency domain measures include Total Power and the LF/HF ratio.

In another aspect, the present invention provides a method of screening a subject for Parkinson's disease or Parkinson's-like disease by measuring cardiac autonomic denervation as one pathway to achieve large scale screening of the general population for Parkinson's disease or Parkinson's-like disease. Cardiac autonomic denervation (CAD) is a near universal feature in Parkinson's disease or Parkinson's-like disease when the motor signs are fully evident. Additionally, CAD may precede motor dysfunction in Parkinson's disease or Parkinson's-like disease as suggested by the presence of Lewy bodies in the superior sympathetic ganglia many years prior to diagnostic Parkinson's disease and in the cardiac plexus in 100% of Parkinson's and incidental Lewy body disease cases. CAD results in reduced HRV and is documented in patients with clinically diagnosable Parkinson's disease. In one embodiment, an easy, non-invasive method of measuring CAD is by heart rate variability (HRV), which can be assessed using a standard electrocardiogram (EKG). Since patients with pre-motor/prodromal Parkinson's disease and/or Parkinson's-like disease may have CAD, this abnormality can be identified by measuring HRV. In some embodiments, HRV is used as a marker to assess RBD. HRV can be measured during wakefulness or during sleep.

In one embodiment, cardiac sympathetic denervation (CSD), a feature in Parkinson's disease, is observed in presymptomatic Parkinson's disease and/or Parkinson's-like disease. In some embodiments, CSD is observed using imaging agents including but not limited to iodine-123 metaiodobenzylguanidine and fluorodopa positron emission tomography imaging and by cardiac catheterization. CSD reduces heart rate variability (HRV), which can be assessed using a standard electrocardiogram (EKG). Reduced HRV is observed in patients with already diagnosed Parkinson's disease. CSD is documented by assessing changes in HRV in a population that has a high probability of having pre-motor Parkinson's disease or Parkinson's-like disease, i.e., patients with RBD. In some embodiments, the present invention's screening method for pre-motor/prodromal Parkinson's disease or Parkinson's-like disease is incorporated into annual physical examinations.

In some embodiments, the subject of the present invention is in a wakeful state or awake while obtaining the EKG result. In some embodiments, the HRV result comprises a frequency domain measure, e.g., Very Low Frequency (VLF) (0-0.04 Hz), Low Frequency (LF) (0.04-0.15 Hz), or High Frequency (HF) (0.15-0.4 Hz). The values can be reported in both absolute values and normalized units. In some embodiments, the frequency domain measures include Total Power and the LF/HF ratio. In some embodiments, the subject has a lower RMSSD than a subject not having an EKG result falling into an EKG result range predetermined to be indicative of Parkinson's disease or Parkinson's-like disease. In some embodiments, the subject has a lower pNN50 than a subject without having an EKG result falling into an EKG result range predetermined to be indicative of Parkinson's disease or Parkinson's-like disease. In some embodiments, the subject has a lower SDNN than a subject without having an EKG result falling into an EKG result range predetermined to be indicative of Parkinson's disease or Parkinson's-like disease. In some embodiments, the subject has a lower SD1 than a subject without having an EKG result falling into an EKG result range predetermined to be indicative of Parkinson's disease or Parkinson's-like disease. In some embodiments, the subject has a lower SD2 than a subject without having an EKG result falling into an EKG result range predetermined to be indicative of Parkinson's disease or Parkinson's-like disease. In some embodiments, the subject has a lower RR triangular index than a subject without having an EKG result falling into an EKG result range predetermined to be indicative of Parkinson's disease or Parkinson's-like disease. In some embodiments, the subject has a lower TINN number than a subject without having an EKG result falling into an EKG result range predetermined to be indicative of Parkinson's disease or Parkinson's-like disease. In some embodiments, the subject has a lower VLF(ms²) than a subject without having an EKG result falling into an EKG result range predetermined to be indicative of Parkinson's disease or Parkinson's-like disease. In some embodiments, the subject has a lower LF(ms²) than a subject without having an EKG result falling into an EKG result range predetermined to be indicative of Parkinson's disease or Parkinson's-like disease. In some embodiments, the subject has a lower HF(ms²) than a subject without having an EKG result falling into an EKG result range predetermined to be indicative of Parkinson's disease or Parkinson's-like disease. In some embodiments, the subject has a lower Total Power(ms²) than a subject without having an EKG result falling into an EKG result range predetermined to be indicative of Parkinson's disease or Parkinson's-like disease.

The methods may further comprise physical examinations, neurological examinations, vital sign measurements, height and weight measurements. These examinations and/or measurements may be performed according to standard practice at the clinical site. Vital signs may include blood pressure (systolic and diastolic), heart rate, temperature, and respiratory rate. Weight may be measured using a calibrated scale with the patient clothed and shoes on. Height may be measured with shoes off using a calibrated wall mounted stadiometer.

The methods may further comprise additional assessments of non-motor symptom changes in heart rate variability and presence of rapid eye movement behavioral sleep disorder (RBSD) and/or REM sleep behavior disorder (RBD). RBD is a parasomnia with loss of muscle atonia during REM sleep resulting in enactment of dreams (Ferini-Strambi et al and Olson et al.) and is associated with alpha-synucleinopathies (Olson et al., Stiasny-Kolster et al. Boeve et al) such as Parkinson's disease or Parkinson's-like disease, Dementia with Lewy Bodies (DLB) and Multiple System Atrophy (MSA). RBD may precede and predict the clinical symptoms of typical Parkinson's disease or Parkinson's-like disease by years to a decade or more. RBD may precede and predict the clinical symptoms of typical Parkinson's disease or Parkinson's-like disease by years to a decade or more. Subjects with REM sleep behavioral disorder (RBD) can have significant alterations in heart rate variability (HRV) as measured by electrocardiogram tracings compared to a group of age matched controls without RBD. In some embodiments, EKG is used to identify changes in HRV in individuals with RBD with possible “pre-motor” or prodromal Parkinson's disease or Parkinson's-like disease.

In some embodiments, the methods comprise undergoes brain imaging. The brain imaging can be PET or MRI.

### Biopsy and Resection Samples

The methods may further comprise obtaining GI biopsy or resection samples from the subject. Lewy bodies in the myenteric plexus of the esophagus and colon suggests that Parkinson's disease may affect the enteric nervous system and contribute to esophageal dysmotility and constipation (Edwards, et al., 1992). Therefore, in the event a patient has a biopsy or resection of any part of the GI tract during the study, the patient will be requested to sign a consent form authorizing The Parkinson's Institute and Clinical Center to receive a portion of the specimen. These specimens will be examined for Lewy neurites in accordance with Lebouvier et al., Colonic biopsies to assess the neuropathology of Parkinson's disease and its relationship with symptoms. PLoS ONE 2010; 5(9): e12728.

### Blood and Urine Samples

The methods may comprise obtaining blood samples. The methods may comprise obtaining urine samples. The methods may comprise obtaining saliva samples. Examinations and measurements may comprise performing clinical laboratory assessments (e.g. chemistry, hematology, and urinalysis). Methods well known in the art may be used to measure blood and/or urine levels of various ions, proteins and macromolecules, including, but not limited to alkaline phosphatase, aspartate aminotransferase, alanine aminotransferase, total bilirubin, total protein, albumin, glucose, carbon dioxide, blood urea nitrogen, creatinine, sodium, potassium, calcium and chloride.

The methods may further comprise collecting blood samples and performing blood cell counts. The blood count may be a complete blood count. The blood count may be a partial blood count. The blood count may be a complete blood count without differential.

### Nucleic Acid and Protein Analysis

The methods disclosed herein may comprise genetic testing for Parkinson's disease or Parkinson's-like disease. Genetic testing may comprise determining if markers associated with the neurological condition are present (e.g., mutations of genes, expression levels of proteins). The methods may further comprise analysis of nucleic acids obtained from the subject (e.g. genetic analysis, gene expression). The analysis of nucleic acids may be carried out by methods well known in the art. The analysis of nucleic acids may comprise a method selected from nucleic acid sequencing, nucleic acid restriction digest, nucleic acid amplification (e.g. PCR), reverse transcriptase PCT (RT-PCR), microarray, gel electrophoresis, fluorescence in situ hybridization, southern blot and northern blot.

The methods may further comprise analysis of proteins obtained from the subject (e.g. protein expression). The analysis of proteins may be carried out by methods well known in the art. The analysis of proteins may comprise a method selected from sodium dimethyl sulfate polyacrylamide gel electrophoresis (SDS-PAGE), enzyme-linked immunosorbent assay (ELISA), western blot, immunohistochemistry and immunofluorescence.

The disorder may be at the early onset stage or the subject may be entirely asymptomatic. For example, to determine if a subject is at risk for Parkinson's disease, the subject can be screened for mutations of one or more LRRK2, α-synuclein, parkin gene or a combination of two or more markers thereof. Furthermore, the subject can be screened for elevated expression levels of a protein indicative of disease onset or risk for disease. Methods of performing such genetic/biochemical screens are known in the art.

In some embodiments, the subject is screened for a mutation in a gene selected from the group consisting of leucine-rich repeat kinase 2 (LRRK2), α-synuclein (SNCA), parkin (PRKN), ubiquitin C-terminal hydrolase L1 (UCH-L1), oncogene DJ-1 gene, PTEN-induced protein kinase 1 (PINK1), and microtubule-associated protein tau (MAPT). Such mutations include but are not limited to substitution, deletion, insertion, duplication, triplication or a combination thereof.

### Leucine-Rich Repeat Kinase 2 (LRRK2) Gene

In one embodiment, the subject is pre-symptomatic of primary symptoms for Parkinson's disease, but genetic screening yields information on the presence mutations and/or polymorphisms of one or more genes associated with Parkinson's disease. For example, a subject is screened for the prevalence of a leucine-rich repeat kinase 2 (LRRK2) gene mutation. Mutations in LRRK2 may be found in both familial and sporadic cases of Parkinson's Disease. In particular, specific mutations encoded by mutant LRRK2 genes that have been proven shown to be pathogenic in the development of Parkinson's Disease include Y1699C, R1441C, R1441H, R1441H, I1371V, Y1699G, G2019S, I2020T, and G2385R. Mutations within LRRK2 that are potentially pathogenic include E334K, Q1111H, I1192V, I1122V, S1228T, A1442P, L1719F, and T2356I. Those mutations that are associated with an increased risk of developing Parkinson's Disease are R1628P and G2385R (see Kumari, ibid). Patients with LRRK2 mutations have shown typical levodopa responsive Parkinson's disease with tremor being the most common presenting feature. Patients with the G2019S mutation have shown a similar age of onset of symptoms when compared with patients with other LRRK2 mutations or sporadic Parkinson's disease, and can be more likely to have a family history of Parkinson's disease. In addition, a familial A1442P (4,324 G>C) mutation has been observed. Therefore, in one embodiment, a subject is tested to determine the presence of LRRK2 mutations and if positive for such mutations, the subject is administered one or more therapies that inhibit, decrease, reverse, or prevents α-synuclein fibrillation and/or aggregation, inhibits MAO, inhibits kinases, blocks calcium channels, enhances mitochondrial function as a prophylactic to delay, reduce or eliminate Parkinson's disease onset or progression. In some cases, patients with a LRRK2 mutation are treated with a LRRK2 modulator (e.g., a LRRK2 inhibitor). In some cases, a LRRK2 inhibitor is selected from the group consisting of PF-06447475, CZC 25146, CZC 54252, GSK2578215A, LRRK2-IN-1, and MLi-2.

### α-Synuclein Gene

The methods may comprise analyzing a protein, and/or a nucleic acid encoding the protein, wherein the protein is selected from alpha-synuclein. α-synuclein is a major constituent of Lewy Bodies, the pathological hallmark of Parkinson's disease. The methods may comprise correlating the extent of assessed symptoms with the levels of α-synuclein and degree of neuronal loss in GI biopsy and surgical resection samples.

The methods may comprise utilizing a genetic screen to detect the presence of α-synuclein gene mutations or multiplications and/or polymorphisms which are major underlying genetic defects known in familial juvenile onset Parkinson's disease.

Mutations in, or over-expression of, α-synuclein may cause damage by interfering with particular steps of neuronal membrane traffic. Alpha-synuclein selectively blocks endoplamic reticulum (ER)-to-Golgi transport, thus causing ER stress. Alpha-synuclein may serve a chaperone function for the proper folding of soluble NSF attachment receptor (SNAREs) that are important for neurotransmitter release.

Therefore in some embodiments, a subject is diagnosed or pronounced to be at-risk after a genetic screen to determine the presence of α-synuclein mutations and/or polymorphisms and/or detection of elevated expression levels of α-synuclein, wherein mutations and/or polymorphisms and/or elevated expression levels are indicative of risk of Parkinson's disease. Further, the subject may be optionally examined for display of one or more secondary symptoms. Thus, in one such embodiment, the subject is administered one or more therapies that inhibit, decrease, reverse, or prevent α-synuclein aggregation and fibrillation and/or aggregation, or inhibits kinases such as LRRK kinase, or inhibits MAO, or acts as a calcium channel blocker, or a mitochondrial enhancer as a prophylactic to delay, reduce or eliminate Parkinson's disease and/or Parkinson's-like disease onset or progression.

In another embodiment, a subject is screened for LRRK2 mutations described above and α-synuclein mutations and/or polymorphisms and/or overexpression, where positive results (e.g., mutations, overexpression) are indicative of risk of developing Parkinson's Disease, and the subject is treated with one or more therapies that inhibit, decrease, reverse, or prevents α-synuclein fibrillation and/or aggregation or inhibits kinases such as LRRK kinase, or inhibits MAO, or acts as a calcium channel blocker, or a mitochondrial enhancer as a prophylactic to delay, reduce or eliminate Parkinson's disease and/or Parkinson's-like disease onset or progression.

### Parkin Gene

In another embodiment, a subject is genetically screened to determine if one or more parkin gene mutation and/or polymorphism is present to determine risk for Parkinson's Disease. If one or more parkin gene(s) are mutated or have a polymorphism associated with a neurological disease then the subject can be treated with a compound named herein. People with one mutation may develop the disease 12 years earlier than average. Two mutated genes are linked with disease which starts 13 years earlier. The prevalence of Parkinson's increases with age—appearing in 1% of people over 60 and 4-5% of those over 85—but it can develop in much younger patients. Inheriting mutations, deletions, or multiplications of the parkin gene is associated with the development of early-onset Parkinson's—which refers to disease which appears before the age of 50.

Therefore, in prophylactic treatment methods of the invention, a subject undergoes genetic screen to determine a risk for Parkinson's disease (e.g., presence of one or more PRKN mutations) and if found to be at-risk, is administered one or more compounds that inhibit, decrease, reverse, or prevent α-synuclein fibrillation and/or aggregation. In some further embodiments, a subject may be screened for PRKN and LRRK2 mutations and/or polymorphisms to determine if a prophylactic administration of one or more therapies described herein that inhibits, decreases, reverses, or prevents α-synuclein fibrillation and/or aggregation, or inhibits kinases such as LRRK kinase, or inhibits MAO, or acts as a calcium channel blocker, or a mitochondrial enhancer as a prophylactic to delay, reduce or eliminate Parkinson's disease and/or Parkinson's-like disease onset or progression is desirable. In any of the genetic screens described herein, the presence of mutations and/or polymorphisms in one familial gene should not serve as exclusion criteria in a screen for further genetic variation.

In certain embodiments, a subject may be routinely screened for mutations and/or polymorphisms, to determine if at risk and determine if a prophylactic administration of one or more compounds described herein that inhibits, decreases, reverses, or prevents α-synuclein fibrillation and/or aggregation, or inhibits kinases such as LRRK kinase, or inhibits MAO, or acts as a calcium channel blocker, or a mitochondrial enhancer as a prophylactic to delay, reduce or eliminate Parkinson's disease and/or Parkinson's-like disease onset or progression is desirable. In other embodiments, a subject may be first screened and secondary non-motor symptoms identified, determined to be at risk, and further screened for mutations and/or polymorphisms to determine if a prophylactic administration of one or more therapies described herein that inhibits, decreases, reverses, or prevents α-synuclein fibrillation and/or aggregation, or inhibits kinases such as LRRK kinase, or inhibits MAO, or acts as a calcium channel blocker, or a mitochondrial enhancer as a prophylactic to delay, reduce or eliminate Parkinson's disease and/or Parkinson's-like disease onset or progression is desirable.

The methods may comprise obtaining nucleic acids or proteins from the subject. The methods may further comprise obtaining deoxyribonucleic acid (DNA) for genetic analysis. The methods may further comprise obtaining ribonucleic acid (RNA) for gene expression analysis. DNA and/or RNA may be obtained from a sample selected from blood, urine, feces, saliva, skin and hair. DNA and/or RNA may be isolated by methods well known in the art (Miller 1988).

### GBA Gene

The GBA gene encodes the lysosomal enzyme, glucocerebrosidase, which is deficient in Gaucher's disease. Gaucher's disease is an autosomal recessive disorder that affects mononuclear phagocyte system and is characterized by lysosomes engorged with stored lipid. Mutations in GBA are common risk factors for Parkinson's disease and related disorders, such as dementia. GBA mutations are associated with varying types of parkinsonian phenotypes and an earlier age of onset, suggesting that mutations in GBA can promote alpha-synuclein aggregation, processing and clearance.

There are three types of Gaucher's disease—non-neuonopathic, acute neuronopathic, and chronis neuronopathic. Non-neuronopathic Gaucher's disease manifest with hepatosplenomegaly, anaemia, thrombocytopenia, and can be treated with enzyme replacement therapy. Acute neuronopathic Gaucher's disease presents early in life with rapidly progressive neurological deterioration. Enzyme replacement therapy can halt neurological progression. Chronic neuronopathic Gaucher's disease includes several phenotypes, including myoclonic epilepsy, cardiac calcification, and hydrocephalus, and other abnormalities.

In some patients, Gaucher manifests with progressive parkinsonian features. Sequencing of GBA identified N370S, E326K, L444P, and T369M variants in some Gaucher patients. In some embodiments, methods comprise analyzing a sample of a patient for a protein and/or a nucleic acid encoding a protein, such as a mutation in any one of GBA, LRRK2, and SNCA, to determine the risk of MLBD or PD. In some cases, methods described herein are used to test a subject with Gaucher disease for a risk of developing MLBD or PD. In some aspects, a subject diagnosed with MLBD or PD is treated with a neuroprotective agent or in combination with a therapeutic agent for Gaucher's disease, such as an enzyme replacement therapy for glucocerebrosidase. In some instances, a chaperone or a carrier molecule is attached to glucocerebrosidase so that it is targeted to a nerve cell or can cross the blood brain barrier to target cells in the brain. In some cases, a patient with one or more symptoms indicative of involvement of peripheral autonomic system as described herein, such as symptoms associated with GI motility or cardiac abnormality, or at least one symptom associated with MLBD or PD, is tested for Gaucher disease, MLBD, and/or PD. In other cases, a subject diagnosed with Gaucher disease or manifests Gaucher symptoms is tested for MLBD and/or PD using a method described herein to determine a risk of developing MLBD or PD. The subject with a high risk of MLBD or PD according to the method described herein is then treated with a neuroprotective agent to slow the progression of nerve damage to the brain. Levels of α-synuclein in nerve cells, such as cells of the enteric nervous system, and/or degree of neuronal loss in GI biopsy and surgical resection samples may be analyzed in a subject at risk for Gaucher disease, MLBD, or PD.

In one embodiment, a subject is tested to determine the presence of GBA mutations and if positive for such mutations, the subject is administered one or more therapies that inhibit, decrease, reverse, or prevents α-synuclein fibrillation and/or aggregation, inhibits MAO, inhibits kinases, blocks calcium channels, enhances mitochondrial function as a prophylactic to delay, reduce or eliminate Parkinson's disease onset or progression. In some cases, patients with a GBA mutation are treated with a GBA modulator, a gene therapy or a cell therapy that provides normal expression of glucocerebrosidase, or an enzyme replacement therapy for glucocerebrosidase, such as imiglucerase, velaglucerase alfa, and taliglucerase alfa. In some cases, a gucosylceramide synthase inhibitor, such as miglustat and eliglustat, is used to treat a subject with or at risk for Gaucher disease, MLBD, or PD.

### Subjects

A subject may be an animal, including but not limited cows, horses, sheep, cats, dogs, pigs, horses, mice, rats, rabbits, squirrels, non-human primates and humans.

Subjects may be patients with GI symptoms. GI symptoms may include, but are not limited to dysphagia, gastroparesis, prolonged GI transit time, constipation, difficulty with defecation, nausea, stomach fullness, vomiting, retching, diarrhea, constipation, (excessive) belching, (excessive) flatulence, heartburn, acid reflux/regurgitation, sucking sensations in the epigastrium, borborygmus, bloating/abdominal extension, eructation, hard stool, loose stool, loss of appetite and abdominal pain.

Subject may be patients with a suspected risk for developing Parkinson's disease. The suspected risk for developing Parkinson's disease may be based on results of the University of Pennsylvania Smell Identification Test (UPSIT), family history, and/or sleep study results.

Subjects may have reported observed Lewy bodies in their brain and/or GI tract. Lewy bodies may be observed in the superior sympathetic ganglia at least 10 years before the diagnosis of Parkinson's disease or Parkinson's-like disease. Furthermore, cardiac Lewy neuritic pathology has been found in most if not all cases of incidental Lewy body cases (presumable Braak Stage I and II Parkinson's disease or prodromal Parkinson's disease).

In some embodiments, the subject being screened by the method of the present invention has not been previously diagnosed as having Parkinson's disease or Parkinson's-like disease. In some embodiments, the subject does not exhibit any motor symptoms indicative of Parkinson's disease or Parkinson's-like disease. In some embodiments, the subject has been assessed to be 0 on the Hoehn and Yahr scale. In some embodiments, the subject has not been assessed on the Hoehn and Yahr scale. In some embodiments, the subject has been assessed to be 0 on the Unified Parkinson's disease rating scale (UPDRS). In some embodiments, the subject has not been assessed on the UPDRS scale. In some embodiments, the subject further has a symptom including but not limited to constipation, olfactory dysfunctions, autonomic disturbances such as dysautonomia, psychological symptoms such as depression, and sleep disorders such as RBD.

The subject may be treated with one or more neuroprotective agents and/or therapies. Neuroprotective agents and therapies may include, but are not limited to, calcineurin inhibitors, NOS inhibitors, sigma-1 modulators, AMPA antagonists, Ca2+ channel blockers. estrogen agonists, glycoprotein IIb/IIIa antagonists, erythropoietin, astaxanthin, boswellia, caffeine, curcumin, E vitamins as tocotrienols, flavonoids, grapefruit juice (naringenin), huperzine, ubiquinol, MAO inhibitors, kinase inhibitors, mitochondrial modulators/enhancers, alpha synuclein modulators and exercise. Some neuroprotective therapies offer protection against cell degeneration to the neuronal cells. Neuroprotective agents may protect the dopamine neurons. Neuroprotective agents may comprise antioxidants. Neuroprotective agents and/or therapies may inhibit, decrease, reverse, or prevent α-synuclein fibrillation and/or aggregation. Neuroprotective agents and/or therapies may induce kinase inhibition. Neuroprotective agents and/or therapies may induce MAO inhibition. Neuroprotective agents and/or therapies may act as a calcium channel blocker. Neuroprotective agents and/or therapies may act as a mitochondrial enhancer. Neuroprotective agents and/or therapies may delay or reduce progression of a neurological condition.

Neuroprotective agents may be selected from levodopa, carbidopa, benserazide and combinations thereof. Levodopa (L-dopa) is used as a form of symptomatic treatment. L-dopa is transformed into dopamine in the dopaminergic neurons by L-aromatic amino acid decarboxylase. However, only 1-5% of L-dopa enters the dopaminergic neurons. The remaining L-dopa is often metabolized to dopamine elsewhere, causing a wide variety of side effects. Due to feedback inhibition, L-dopa results in a reduction in the endogenous formation of L-dopa, and so eventually becomes counterproductive. Carbidopa and benserazide are dopa decarboxylase inhibitors. They help to prevent the metabolism of L-dopa before it reaches the dopaminergic neurons and are generally given as combination preparations of carbidopa/levodopa (co-careldopa) and benserazide/levodopa (co-beneldopa). Duodopa is a combination of levodopa and carbidopa.

Neuroprotective agents may be dopamine agonists. The dopamine agonists may be selected from bromocriptine, pergolide, pramipexole, ropinirole, cabergoline, apomorphine and lisuride. Dopamine agonists may be useful for patients experiencing on-off fluctuations and dyskinesias as a result of high doses of L-dopa.

Neuroprotective agents may be MAO-B inhibitors (first, second, or later generation MAO-B inhibitors). MAO-B inhibitors may reduce the symptoms associated with Parkinson's disease by inhibiting the breakdown of dopamine secreted by the dopaminergic neurons. An exemplary MAO-B inhibitor is Rasagiline [N-propargyl-1(R)-aminoindan], a second-generation propargylamine pharmacophore that selectively and irreversibly inhibits brain MAO-B.

Neuroprotective agents may be noradrenergic drugs (e.g. norepinephrine). Noradrenergic drugs may be useful in preventing, reversing, or treating early premotor/prodromal Parkinson's disease or Parkinson's-like disease.

Neuroprotective agents may be kinase inhibitors, such as p38 mitogen-activated protein kinase inhibitors, mixed lineage kinase inhibitors, (for example CEP-1347) and Leucine-rich Repeat Kinase 2 (LRRK2) inhibitors. Kinase inhibitors may be useful in preventing, reversing, or treating early premotor/prodromal Parkinson's disease or Parkinson's-like disease.

Neuroprotective agents may be mitochondrial modulators (e.g. Enzyme co-Q10), which may be useful in preventing, reversing, or treating early premotor/prodromal Parkinson's disease or Parkinson's-like disease.

Neuroprotective agents may be calcium channel blockers (e.g. isradipine), which may be useful in preventing, reversing, or treating early premotor/prodromal Parkinson's disease or Parkinson's-like disease.

Increased exercise may be useful in preventing, reversing, or treating early premotor/prodromal Parkinson's disease or Parkinson's-like disease.

Compounds that prevent/reverse/disaggregate, halt aggregation of alpha-synuclein may be useful in preventing, reversing, or treating early premotor/prodromal Parkinson's disease or Parkinson's-like disease. Such compounds are described and listed in WO/2009/003147, the publication is hereby incorporated in its entirety.

In some embodiments, a subject who has been diagnosed to have prodromal or pre-motor Parkinson's disease or Parkinson's-like disease using the method of the present invention can be treated with a prophylactic drug or other therapy such as exercise. A prophylactic drug for Parkinson's disease or Parkinson's-like disease is a drug taken to maintain health and prevent or delay the onset of Parkinson's disease or Parkinson's-like disease. For example, such subject can be administered a compound that inhibits, decreases, reverses, or prevents α-synuclein fibrillation and/or aggregation as a prophylactic measure. In other embodiments, such subject can be given gene therapy. For example, an adeno-associated virus can be used to transport a gene that codes for the enzyme glutamic acid decarboxylase (GAD) into the neurons of the subthalamic nucleus. The gene prompts these subthalamic cells to produce gamma-aminobutyric acid (GABA), the brain's primary inhibitory neurotransmitter, which decreases the activity in the subthalamic nucleus, a brain area that tends to be extremely overactive in Parkinson's patients, thereby restoring the normal motor function. Other experimental techniques for treatment of neurodegenerative disorders include stem cells transplants and upregulation of a molecule that prevents neurodegeneration.

### Kits

In yet another aspect, the present invention provides kits for carrying out the methods of the present invention. The kits may include materials to test for the predisposition of a neurological disorder, e.g. Parkinson's disease or Parkinson's-like disease. In some embodiments, the kits include reagents and instruments for measuring EKG of a subject undergoing the screening for Parkinson's disease or Parkinson's-like disease. The kits may further comprise suitable packaging, and written material that can include instructions for use, discussion of clinical studies, listing of side effects, and the like. The kits may further contain a neuroprotective agent. The kits may further include material for olfactory testing. The reagents, instruments and other agents may be provided as separate or individual compositions and/or devices within the kit. Kits may include any combination of the following: tools for performing an esophageal and/or anorectal manometry, a SmartPill®, a G-Tech monitoring device, a GI Symptom Relief Scale (GSRS), a Gastroparesis Cardinal Symptom Index (GCSI), a UPSIT, a Hoehn Yahr Scale, a UPDRS scale, tools for collecting a tissue/fluid sample, reagents for nucleic acid and/or protein purification and oligonucleotides and/or antibodies for nucleic acid and/or protein detection. Oligonucleotides and/or antibodies may be specific for genetic mutations in a gene of interest (e.g. parkin, alpha-synuclein, LRRK2). In some embodiments, the methods comprise identifying patients early in the disease process/progression. In some embodiments, the methods comprise identifying patients in the “prodromal/premotor” stage(s) of disease progression. In some embodiments, the methods comprise identifying patients in the stage(s) preceding the development of motor symptoms, i.e. premotor MLBD and/or PD. In some embodiments, the methods comprise identifying patients in the early stages of disease progression before substantial cell loss has occurred in the brain

In some embodiments, the methods comprise instituting directing, implementing disease-modifying interventions after identifying the patients in the premotor stages of disease progression. In some embodiments, the methods comprise administering treatment to slow disease progression. In some embodiments, the treatment comprises the monoamine oxidase (MAO) inhibitors, selegiline, rasagiline, or any combination thereof. In some embodiments, the disease-modifying intervention comprises an intensive exercise program. In certain embodiments, the intensive exercise program can delay the need for treatment. In some cases, the intensive exercise program can delay the need for treatment with L-dopa.

In one aspect, the present disclosure presents methods and systems that use parts of the GI system and/or enteric nervous system as model to study disease progression of MLBD and/or PD and to develop treatments.

In some embodiments, the methods and/or systems comprise using parts of the GI system and/or enteric nervous system as model to study the early premotor stage(s) of MLBD and/or PD

In some embodiments, the methods and/or systems comprise developing treatments that can prevent α-synuclein accumulation in the GI tract and/or ENS. In some embodiments, the treatments can prevent α-synuclein accumulation in the brain.

In some embodiments, the methods and/or systems comprise characterizing GI symptoms in MLBD and/or PD. In some embodiments, the methods and/or systems comprise characterizing GI symptoms in “premotor” MLBD and/or PD. In some embodiments, the said characterizing comprises determining GI function. In some embodiments, GI function is determined using qualified, sensitive, and/or quantitative instruments of GI function. In some embodiments, the methods and/or systems further comprise correlating GI symptoms with MLBD and/or PD motor symptoms and other non-motor symptoms.

In some embodiments, determining GI function(s) comprises using GI diagnostics. In some embodiments, the GI diagnostics comprise evaluation of GI abnormalities. In some embodiments, the GI diagnostics comprise High Resolution Esophageal Manometry (HREM), High Resolution Anorectal Manometry (HRAM), Wireless Motility Capsule (WMC) [also known as SmartPill], or any combination thereof.

In some embodiments, the GI diagnostics comprises symptom-based assessment(s). In some embodiments, the symptom-based assessments have been validated by a regulatory agency. In some embodiments, the symptom-based assessment(s) comprise assessment(s) used for GI therapeutic regulatory approval and/or early disease diagnosis. In some embodiments, the symptom-based assessment(s) comprises the GI Symptom Relief Scale (GSRS), Gastroparesis Cardinal Symptom Index (GCSI), or any combination thereof.

### Methods of Diagnosis and Treatment

Also provided herein are methods of determining a risk of developing a neurological disease or disorder in an individual, the method comprising: a) determining a sequence in a nucleic acid sample obtained from the individual; b) assessing a peripheral autonomic nervous system response in the individual; and c) determining that the individual has a high risk of developing the neurological disease or disorder if the sequence has a mutation and the individual has an impaired autonomic nervous system response or d) determining that the individual has a low risk of developing the neurological disease or disorder if either the sequence does not have a mutation or the individual has a normal autonomic nervous system response. In some embodiments, the neurological disease or disorder is selected from the group consisting of multisystem Lewy body disease, Parkinson's disease, and Parkinsonism. In some embodiments, the neurological disease or disorder is multisystem Lewy body disease. In some embodiments, the mutation is in a gene selected from one or more of the group consisting of LRRK2, GBA, SNCA, VPS35, DJ-1, PINK1, PARK2, and DNAJ13C. In some embodiments, the mutation is in a gene selected from one or more of the group consisting of LRRK2, GBA, and SNCA. In some embodiments, the peripheral autonomic nervous system response is selected from one or more of the group consisting of GI function, olfactory function, sleep disorder, and cardiac function. In some embodiments, GI function is measured by one or more of the group consisting of esophageal manometry, anorectal manometry, wireless motility capsule, GI symptom questionnaires. In some embodiments, olfactory function is measured by University of Pennsylvania Smell Identification Test (UPSIT). In some embodiments, the sleep disorder is rapid eye movement behavioral sleep disorder (RBSD). In some embodiments, the cardiac function is measured by a method selected from one or more of the group consisting of cardiac MIBG scintigraphy scan, EKG scan, iodine-123 metaiodobenzylguanidine and fluorodopa positron emission tomography imaging, and cardiac catheterization. In some embodiments, the method further comprises administering a modulator of a gene selected from one or more the group consisting of LRRK2, GBA, SNCA, VPS35, DJ-1, PINK1, PARK2, and DNAJ13C. In some embodiments, the modulator is an inhibitor. In some embodiments, the inhibitor selected from one or more of the group consisting of an antibody, an antisense nucleic acid, and a small molecule inhibitor. In some embodiments, the modulator is an agonist. In some embodiments, the agonist is selected from the group consisting of an enzyme replacement therapy, a peptide, and a small molecule agonist. In some embodiments, the mutation is in LRRK2 and the modulator is a LRRK2 inhibitor. In some embodiments, the mutation is in GBA and the modulator is glucocerebrosidase replacement therapy or gucosylceramide synthase inhibitor. In some embodiments, the glucocerebrosidase replacement therapy is administered with a chaperone that facilitates crossing the blood brain barrier. In some embodiments, the mutation is in SNCA, and the modulator is an inhibitor of SNCA expression. In some embodiments, the inhibitor of SNCA expression is selected from the group consisting of an SNCA antisense nucleic acid, an SNCA siRNA, an SNCA shRNA, and an SNCA antibody. In some embodiments, the method further comprises administering one or more of the group consisting of L-dopa, monoamine oxidase B inhibitor, dopamine agonist, catechol-O-methyltransferase inhibitor, anticholinergic, and amantadine.

Also provided herein are methods of determining a risk of developing a multisystem Lewy body disease in an individual, the method comprising: a) determining a sequence in a nucleic acid sample obtained from the individual; b) assessing a peripheral autonomic nervous system response in the individual; and c) determining that the individual has a high risk of developing the neurological disease or disorder if the sequence has a mutation and the individual has an impaired autonomic nervous system response or d) determining that the individual has a low risk of developing the neurological disease or disorder if either the sequence does not have a mutation or the individual has a normal autonomic nervous system response. In some embodiments, the mutation is in a gene selected from one or more of the group consisting of LRRK2, GBA, SNCA, VPS35, DJ-1, PINK1, PARK2, and DNAJ13C. In some embodiments, the mutation is in a gene selected from one or more of the group consisting of LRRK2, GBA, and SNCA. In some embodiments, the peripheral autonomic nervous system response is selected from one or more of the group consisting of GI function, olfactory function, sleep disorder, and cardiac function. In some embodiments, GI function is measured by one or more of the group consisting of esophageal manometry, anorectal manometry, wireless motility capsule, GI symptom questionnaires. In some embodiments, olfactory function is measured by University of Pennsylvania Smell Identification Test (UPSIT). In some embodiments, the sleep disorder is rapid eye movement behavioral sleep disorder (RBSD). In some embodiments, the cardiac function is measured by a method selected from one or more of the group consisting of cardiac MIBG scintigraphy scan, EKG scan, iodine-123 metaiodobenzylguanidine and fluorodopa positron emission tomography imaging, and cardiac catheterization. In some embodiments, the method further comprises administering a modulator of a gene selected from one or more the group consisting of LRRK2, GBA, SNCA, VPS35, DJ-1, PINK1, PARK2, and DNAJ13C. In some embodiments, the modulator is an inhibitor. In some embodiments, the inhibitor selected from one or more of the group consisting of an antibody, an antisense nucleic acid, and a small molecule inhibitor. In some embodiments, the modulator is an agonist. In some embodiments, the agonist is selected from the group consisting of an enzyme replacement therapy, a peptide, and a small molecule agonist. In some embodiments, the mutation is in LRRK2 and the modulator is a LRRK2 inhibitor. In some embodiments, the mutation is in GBA and the modulator is glucocerebrosidase replacement therapy or gucosylceramide synthase inhibitor. In some embodiments, the glucocerebrosidase replacement therapy is administered with a chaperone that facilitates crossing the blood brain barrier. In some embodiments, the mutation is in SNCA, and the modulator is an inhibitor of SNCA expression. In some embodiments, the inhibitor of SNCA expression is selected from the group consisting of an SNCA antisense nucleic acid, an SNCA siRNA, an SNCA shRNA, and an SNCA antibody. In some embodiments, the method further comprises administering one or more of the group consisting of L-dopa, monoamine oxidase B inhibitor, dopamine agonist, catechol-O-methyltransferase inhibitor, anticholinergic, and amantadine.

Also provided herein are methods of treating a multisystem Lewy body disease in an individual, the method comprising: a) determining a sequence in a nucleic acid sample obtained from the individual; b) assessing a peripheral autonomic nervous system response in the individual; and c) administering a treatment selected based a mutation found in the nucleic acid sample. In some embodiments, the mutation is in a gene selected from one or more of the group consisting of LRRK2, GBA, SNCA, VPS35, DJ-1, PINK1, PARK2, and DNAJ13C. In some embodiments, the mutation is in a gene selected from one or more of the group consisting of LRRK2, GBA, and SNCA. In some embodiments, the peripheral autonomic nervous system response is selected from one or more of the group consisting of GI function, olfactory function, sleep disorder, and cardiac function. In some embodiments, GI function is measured by one or more of the group consisting of esophageal manometry, anorectal manometry, wireless motility capsule, GI symptom questionnaires. In some embodiments, olfactory function is measured by University of Pennsylvania Smell Identification Test (UPSIT). In some embodiments, the sleep disorder is rapid eye movement behavioral sleep disorder (RBSD). In some embodiments, the cardiac function is measured by a method selected from one or more of the group consisting of cardiac MIBG scintigraphy scan, EKG scan, iodine-123 metaiodobenzylguanidine and fluorodopa positron emission tomography imaging, and cardiac catheterization. In some embodiments, the treatment comprises administering a modulator of a gene selected from one or more the group consisting of LRRK2, GBA, SNCA, VPS35, DJ-1, PINK1, PARK2, and DNAJ13C. In some embodiments, the modulator is an inhibitor. In some embodiments, the inhibitor selected from one or more of the group consisting of an antibody, an antisense nucleic acid, and a small molecule inhibitor. In some embodiments, the modulator is an agonist. In some embodiments, the agonist is selected from the group consisting of an enzyme replacement therapy, a peptide, and a small molecule agonist. In some embodiments, the mutation is in LRRK2 and the modulator is a LRRK2 inhibitor. In some embodiments, the mutation is in GBA and the modulator is glucocerebrosidase replacement therapy. In some embodiments, the glucocerebrosidase replacement therapy is administered with a chaperone that facilitates crossing the blood brain barrier. In some embodiments, the mutation is in SNCA, and the modulator is an inhibitor of SNCA expression. In some embodiments, the inhibitor of SNCA expression is selected from the group consisting of an SNCA antisense nucleic acid, an SNCA siRNA, an SNCA shRNA, and an SNCA antibody. In some embodiments, the method further comprises administering one or more of the group consisting of L-dopa, monoamine oxidase B inhibitor, dopamine agonist, catechol-O-methyltransferase inhibitor, anticholinergic, and amantadine.

### EXAMPLES

### Example 1: MLBD Protein Interaction Networks

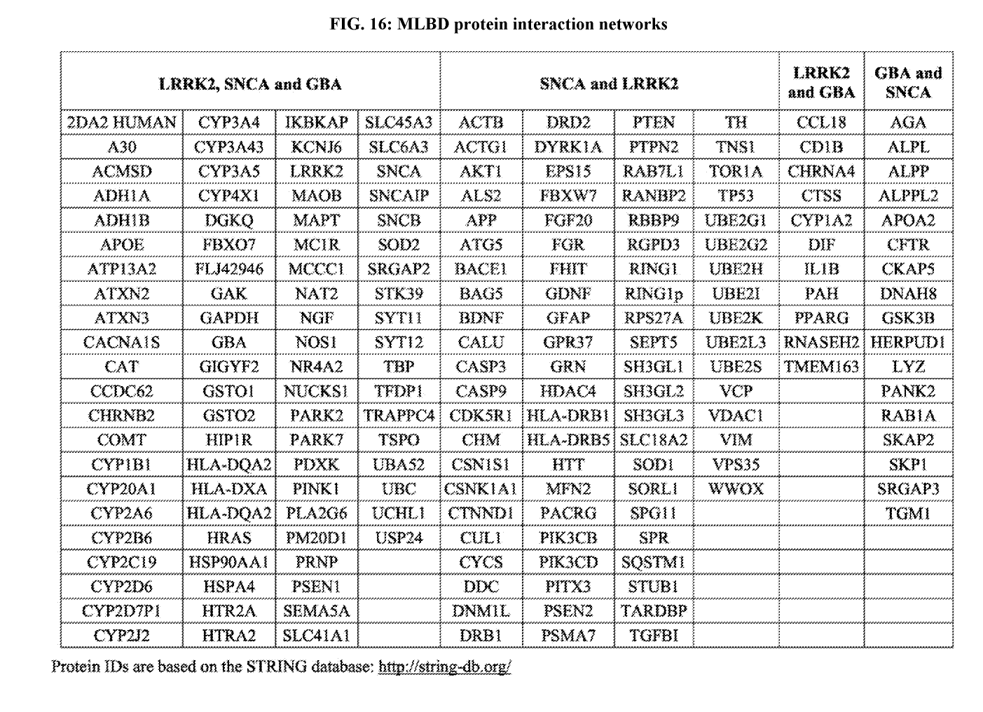

For purposes of illustrating this approach in some embodiments, interrogating the public database STRING DB (48) can drive understanding of the best next steps toward identifying gene products as novel therapeutic targets. Using the information in TABLE 1 (see also FIG. 10), interacting proteins from among the products of the first group of genes were identified, which are a mix of disease alleles that are associated with some aspects of MLBD and several genes that are associated with parkinsonism but for which there is little or no data to support their association with a primary Lewy body pathology diagnosis (LRRK2, GBA, SNCA, VPS35, DJ-1, PINK1, PARK2 and DNAJC13). There were multiple interactions with these proteins, but only a single interaction was found in common among the eight: human ubiquitin C (FIG. 2; UBC). When the protein interaction network search was limited to only genes that are associated with MLBD or possible MLBD (LRRK2, DNAJC13, GBA, and PINK1), only two common interacting proteins, UBC and Hsp70 (HSPA4), were found (FIG. 3). However, the interaction network for proteins encoded by genes best characterized to cause MLBD (LRRK2, SNCA and GBA) showed an extensive overlap in the number of common interactions (more than 50, FIG. 4 and FIG. 16; see TABLE 7 for a list of these interactions; see also FIG. 16). Although databases have inherent bias based on complexities ranging from the definition of the protein-protein interaction to the number of people studying the genes (49) (which can be difficult to correct for and whose statistical effect remains controversial), it is still useful to consider this approach. In some embodiments, it is difficult to account for the potential inherent bias in the data sets, and for the additional potential bias resulting from reducing the number of ‘interactors’ from five to three; nonetheless, these findings are consistent with clinical analyses of phenotype. In some embodiments, although the interaction network for the proteins encoded in this set of genes is extensive and requires validation, conceptually, these networks illustrate how this approach could provide insights that cannot be gained from single-protein analyses. Taken together, these illustrations highlight the potential importance of defining the underlying pathological process to understanding genetic diseases and their relevance to corresponding sporadic diseases in some embodiments.

Table 7: MLBD Protein Interaction Networks, with More than 50 Overlapping Interactions.

TABLE 7
MLBD protein interaction networks, with more than 50 overlapping interactions.

| | | | | | | | | LRRK2 | GBA and |
|---|---|---|---|---|---|---|---|---|---|
| LRRK2, SNCA and GBA | | | | SNCA and LRRK2 | | | | and GBA | SNCA |
| 2DA2_HUMAN | CYP3A4 | IKBKAP | SLC45A3 | ACTB | DRD2 | PTEN | TH | CCL18 | AGA |
| A30 | CYP3A43 | KCNJ6 | SLC6A3 | ACTG1 | DYRK1A | PTPN2 | TNS1 | CD1B | ALPL |
| ACMSD | CYP3A5 | LRRK2 | SNCA | AKT1 | EPS15 | RAB7L1 | TOR1A | CHRNA4 | ALPP |
| ADH1A | CYP4X1 | MAOB | SNCAIP | ALS2 | FBXW7 | RANBP2 | TP53 | CTSS | ALPPL2 |
| ADH1B | DGKQ | MAPT | SNCB | APP | FGF20 | RBBP9 | UBE2G1 | CYP1A2 | APOA2 |
| APOE | FBXO7 | MC1R | SOD2 | ATG5 | FGR | RGPD3 | UBE2G2 | DIF | CFTR |
| ATP13A2 | FLJ42946 | MCCC1 | SRGAP2 | BACE1 | FHIT | RING1 | UBE2H | IL1B | CKAP5 |
| ATXN2 | GAK | NAT2 | STK39 | BAG5 | GDNF | RING1p | UBE2I | PAH | DNAH8 |
| ATXN3 | GAPDH | NGF | SYT11 | BDNF | GFAP | RPS27A | UBE2K | PPARG | GSK3B |
| CACNA1S | GBA | NOS1 | SYT12 | CALU | GPR37 | SEPT5 | UBE2L3 | RNASEH2 | HERPUD1 |
| CAT | GIGYF2 | NR4A2 | TBP | CASP3 | GRN | SH3GL1 | UBE2S | TMEM163 | LYZ |
| CCDC62 | GSTO1 | NUCKS1 | TFDP1 | CASP9 | HDAC4 | SH3GL2 | VCP | | PANK2 |
| CHRNB2 | GSTO2 | PARK2 | TRAPPC4 | CDK5R1 | HLA-DRB1 | SH3GL3 | VDAC1 | | RAB1A |
| COMT | HIP1R | PARK7 | TSPO | CHM | HLA-DRB5 | SLC18A2 | VIM | | SKAP2 |
| CYP1B1 | HLA-DQA2 | PDXK | UBA52 | CSN1S1 | HTT | SOD1 | VPS35 | | SKP1 |
| CYP20A1 | HLA-DXA | PINK1 | UBC | CSNK1A1 | MFN2 | SORL1 | WWOX | | SRGAP3 |
| CYP2A6 | HLA-DQA2 | PLA2G6 | UCHL1 | CTNND1 | PACRG | SPG11 | | | TGM1 |
| CYP2B6 | HRAS | PM20D1 | USP24 | CUL1 | PIK3CB | SPR | | | |
| CYP2C19 | HSP90AA1 | PRNP | | CYCS | PIK3CD | SQSTM1 | | | |
| CYP2D6 | HSPA4 | PSEN1 | | DDC | PITX3 | STUB1 | | | |
| CYP2D7P1 | HTR2A | SEMA5A | | DNM1L | PSEN2 | TARDBP | | | |
| CYP2J2 | HTRA2 | SLC41A1 | | DRB1 | PSMA7 | TGFBI | | | |

### Example 2: Calculating Euclidean Distances from Idiopathic Parkinson's Disease

For purposes of illustrating this approach in some embodiments, euclidean distances from idiopathic Parkinson's disease was calculated based on 29 factors for the 22 genetic forms (see TABLE 6 for data on the distance metrics; see also FIG. 15). Data were plotted versus average age of onset for all variants. The size of each circle represents the relative frequency of the genetic forms. To aid in visualization, the radius of the circles are scaled relative to the most common form by subtracting the log 2 value of the observed prevalence from the log 2 value of the least-common form. These factors allowed for easier visualization of the relative frequency over the observed range. However, it should be noted that the scale is not linear with respect to the actual observed frequencies and the reader is referred to TABLE 1 (see also FIG. 10) for more detail. Because there are varying degrees of robustness of neuropathological data, grayscale shading was used to reflect this as follows: darkest shade indicates Lewy body pathology in all cases; medium shading indicates variable findings with the majority of cases showing Lewy body pathology; light shading indicates Lewy body pathology in only a few cases; lightest shading indicates Lewy body pathology was not found but the data is sparse or incomplete, or no data were available. The area between the grey hashed lines indicates early-onset parkinsonism and below the bottom grey hash line, juvenile (<20y) parkinsonism.

### Example 3: Visualizing MLBD Protein Interaction Networks

For purposes of illustrating this approach in some embodiments, STRING DB and exported protein interaction network for all human proteins were accessed. Protein interaction networks from this data for three groupings of genes are found in TABLE 1 (see also FIG. 10). HUGO terms in STRING DB were used to identify the gene products and interactors. Knowledge Explorer™ (available from 10 Informatics) was used to visualize the protein interaction network from STRING DB for the gene products of interest (FIGS. 2-4). The list of proteins that interact with the highly validated MLBD associated genes LRRK2, GBA, SNCA are shown in FIG. 4 and can be found in TABLE 7 and FIG. 16. The full list of protein interactions for genes with mutations that are known to cause parkinsonism but do not always manifest with the same neuropathological findings are shown in FIG. 2 (LRRK2, GBA, SNCA, VPS35, DJ-1, PINK1, PARK2 and DNAJ13C), and can be found at http://www.thepi.org/scientific-resources/.

Correlation of GI Symptoms with PD

For purposes of illustrating this approach in some embodiments, a retrospective study of PD patients was conducted to show that GI symptoms correlated with PD. A total cohort of 95 PD patients was evaluated for various GI symptoms, predominantly constipation, dysphagia, nausea, early satiety, malnutrition and weight loss. Of those evaluated for constipation, all had previously undergone colonoscopy or sigmoidoscopy without revealing any structural explanation for their constipation. GI symptoms were recorded using a simple questionnaire at the GI clinic. Briefly, symptoms were scored for dysphagia, odynophagia, heartburn, regurgitation, chest pain, bloating and weight loss as: (0=no symptom, 1=mild symptom, 2=moderate symptom, and 3=severe symptom), and then by frequency (0=once a week, 1=2 to 6 times a week, 2=7 to 15 times a week, and 3=more than 15 times a week). Weight loss was further quantified as: (0=no weight change, 1=weight loss <10 lbs, 2=weight loss >11-20 lbs and 3=weight loss >20 lb. For lower GI symptoms, scores were graded as reduced frequency of bowel evacuation (<3/week), straining at evacuation, sensation of incomplete evacuation and fecal incontinence (0=no symptom, 1=mild symptom, 2=moderate symptom, 3=severe symptom), and then by frequency (0=once a week, 1=2 to 6 times a week, 2=7 to 15 times a week, and 3=more than 15 times a week). Neurological assessment was made using the Hoehn and Yahr scale (score 0-5) and duration of PD was recorded in years since diagnosis. Variable PD therapies were used and not discontinued for the tests.

### Example 4: Esophageal Dysfunction in PD

Dysphagia is a common problem in PD; its etiology is multifactorial and its management challenging. In this retrospective cohort analysis, the objective was to characterize dysphagia and/or other esophageal symptoms in PD, assess the prevalence of outflow obstruction and major or minor disorders of esophageal peristalsis leading to impaired esophageal clearance and highlight objective parameters that can help in the current management algorithm. Thirty-three patients with PD presenting with dysphagia, odynophagia, heartburn, regurgitation, chest pain and weight loss underwent clinical and functional evaluation by HREM. Esophago-gastric junction (EGJ) outflow obstruction and disorders of peristalsis were assessed using the Chicago classification v3. The remaining patients were unwilling or unable to undergo HRM or did not have any symptoms consistent with esophageal dysfunction. Their median Hoehn and Yahr score was 2.8 (range 1.5-5); median duration of their PD was 8.5 years (range 3-20). The median age of the patients was 70 years (range 53-89 years), 24 (75%) were men and their median cumulative symptom score was 0.36.

Symptoms were prevalent in the 33 patients studied. The majority (62%) experienced dysphagia, likely contributing to weight loss in 41%. Odynophagia was rare (6%) while gastroesophageal reflux (GER) symptoms, such as heartburn, regurgitation and chest pain were noted in 37%, 31% and 28% of patients respectively. The prevalence of symptoms was not significantly different, as compared to the 62 patients who were not studied (data not shown). The prevalence of dysphagia in the 95 patients with PD was 45%, contributing to weight loss in 22%. In terms of clinical severity, dysphagia, chest pain and weight loss were the most prominent symptoms. There was no relationship between PD severity or duration and GI symptom scores.

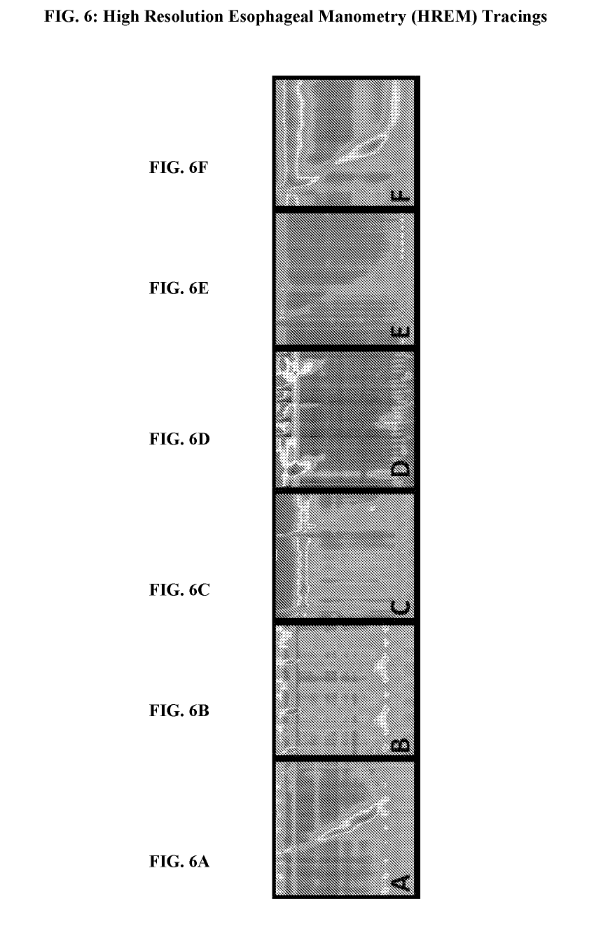

Based on HREM measurements, the median lower esophageal sphincter pressure (LESP) was 28 mmHg (range 11-73 mmHg; IQR 24-37). Four patients had hypotonicity, while 5 had hypertonicity of the LES. Thirteen had elevated residual pressures (RP) upon swallowing (median pressure: 33 mmHg; IQR 22-51), suggestive of EGJ obstruction; 5 were associated with elevated LESP. Five patients had manometrically discernable hiatal hernias, ranging from 0.5-2.2 cm in length. Sixteen patients had abnormal prevalence of premature contractions (>20%), 9 had >30% simultaneous contractions, 14 had more than 20% small breaks, 11 had >20% large breaks, and 18 had >30% failed peristalsis. Eight had distal contractile integral (DCI)<450 mmHg.s.cm. FIG. 6 depicts representative HRM images of abnormalities. FIG. 6A shows EGJ obstruction; FIG. 6B shows pan-esophageal pressurization; FIG. 6C reveals diffuse esophageal spasm; FIG. 6D shows fragmented peristalsis (large breaks); and FIG. 6E shows ineffective esophageal peristalsis; and FIG. 6F shows tracings for normal patient. There was no correlation between severity of manometric findings and PD severity or duration. In a subset of patients (n=16) who had concomitant impedance measurements, incomplete esophageal clearance was noted in all, ranging from 30% to 100% (median 62%; IQR 42-83).

The classification of esophageal dysfunction was determined using the Chicago classification v.3. Twelve patients (39%) exhibited esophago-gastric junction (EGJ) outflow obstruction, 16 (48%) diffuse esophageal spasm (DES), 18 (55%), ineffective esophageal peristalsis (IEM), 16 (48%) fragmented peristalsis, and only 2 patients (6%) had normal HRM tracings. There were no patients with HRM features of achalasia.

The studies indicate that dysphagia is common in patients with PD and is associated with a high prevalence of underlying motility disturbances as identified by HRM.

### Example 5: Measuring Constipation in PD Patients

The etiology of constipation in PD remains poorly understood. Defecatory dyssynergia, anal sphincter spasticity, and slow transit constipation may, individually or collectively, play a role. Radiologic assessment of colonic transit using Sitzmarks and the assessment of anorectal pressures and sensation by anorectal manometry (ARM), including ARM based balloon expulsion test (BET), are important clinical tools for the diagnosis of slow transit constipation, dyssynergic defecation, and fecal incontinence in patients who do not respond to conservative therapy. In lieu of Sitzmarks study, recent guidelines have supported the WMC or SmartPill for the evaluation of colonic transit in chronic constipation, while HRAM including BET are being increasingly used for the diagnosis of dyssynergic defecation and fecal incontinence, instead of conventional ARM. HRAM provides greater resolution, minimizes artifacts, and generates three-dimensional topographical plots of intraluminal pressure profiles, increasing the diagnostic accuracy of anorectal dysfunction. The WMC or SmartPill is an ambulatory non-invasive and non-radioactive diagnostic sensor that continuously samples intraluminal pH, temperature, and pressure as it moves through the GI tract. Studies have shown that the estimated inter-subject coefficients of variation in healthy and constipated subjects are 1 and 0.99 respectively. This technology has permitted routine quantification of transit in all gut regions in a single test and it has been increasingly used for the diagnosis of slow-transit constipation. Used together, WMC, HRAM with BET are poised to guide optimal therapy for functional anorectal disorders in the general population as well as in special groups, such as in patients with PD. The utility of HRAM, balloon expulsion and WMC testing in defining the underlying etiology for constipation in PD was studied. Of the 95 patients, 66 patients fulfilling Rome IV criteria for functional constipation were evaluated. Most patients (89%) had abnormal manometry, exhibiting various types of defecatory dyssynergia (mostly types II and IV), abnormal balloon expulsion, diminished rectal sensation and, in some, lacking recto-anal inhibitory reflex. Sixty-two percent exhibited colonic transit delay by WMC study, while 57% had combined manometric and transit abnormalities, suggestive of “overlap constipation”. Symptoms of infrequent defecation, straining, and incomplete evacuation were not discriminatory.

There was a relationship between constipation scores and colonic transit times (p<0.01); neither PD stage nor duration of disease were correlated with either the manometric or transit findings. Fecal incontinence was seen in 26% of the patients. Median age of the 66 patients in the study was 71 years (range 52-91 years), and 26 (39%) were women. Median Hoehn and Yahr score was 3 (95% median CI: 2.72, 3.00); median duration of PD was 8.5 years (range 3-20). The mean scores for the individual symptoms assessed by questionnaires were: bloating, 1.05 (95% median CI: 0.00, 1.00), constipation, 1.91 (95% median CI: 1.00, 3.00), straining at defecation, 1.83 (95% median CI: 1.98, 2.00), incomplete evacuation, 1.34 (95% median CI: 1.00, 2.00), and fecal incontinence 0.41 (95% median CI: 0.00, 0.00).

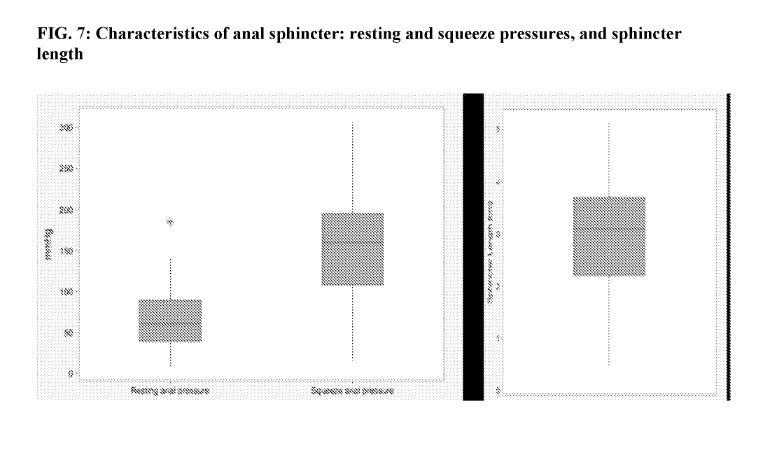

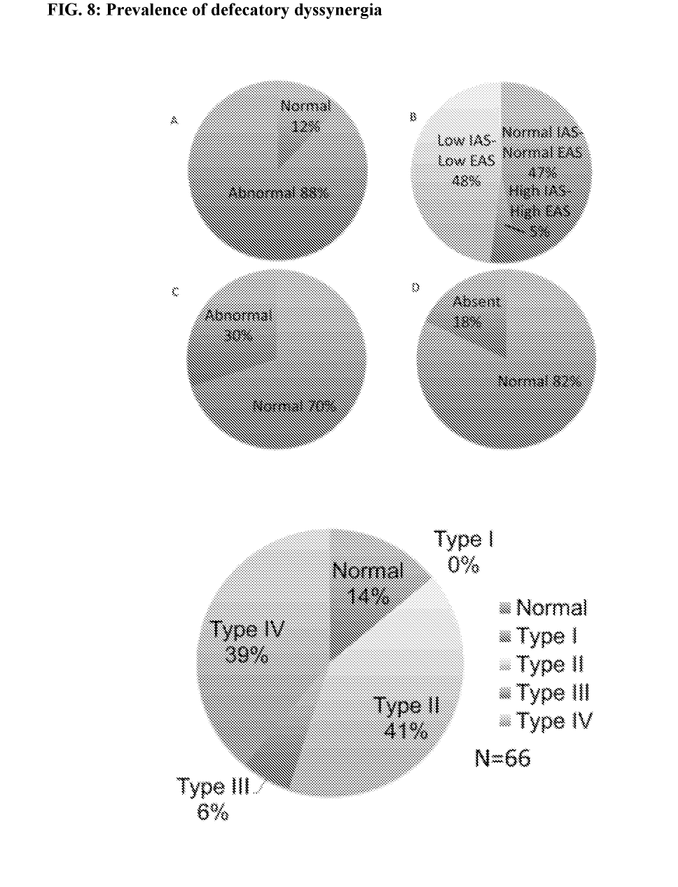

Based on high resolution anorectal manometry (HRAM), the median anal sphincter length was 3.1 cm (95% CI: 2.79, 3.30). FIG. 7 depicts box plot graphs highlighting resting and squeeze anal sphincter pressures in mmHg (left panel) and sphincter lengths in cm (right panel). By HRAM, decreased anal resting pressure was noted in 38 patients (58%), increased in 14 (21%), and normal in 14 (21%). Decreased anal squeeze pressure was noted in 50 patients (76%), increased in 7 (11%) and normal in 9 (14%) patients, the median anal resting pressure and squeeze pressure were 61 mmHg (95% CI: 52.83, 74.01) and 160 mm Hg (95% CI: 140.90, 169.01), respectively. The median rectal pressure and anal pressure during simulated evacuation were 18 mm Hg (95% CI: 12.9, 22) and 75 mm Hg (95% CI: 63.9, 86), respectively. The median percentage of anal relaxation was 5 (95% CI: 1.9, 7.00). The median first sensation was elicited with 60 cc of balloon distention (95% CI: 60.0, 60.0). During BET, only 8 patients (12%) were able to expel the balloon in 1 minute; normal is under 1 minute.

The pie charts of composite FIG. 8 highlight several HRAM characteristics of the study cohort. The top panel shows composite figure (pie charts) highlighting several HRAM characteristics of the cohort—A: Balloon expulsion test; B: Percent prevalence of certain anal sphincter measurements, such as low internal anal sphincter (IAS) and low external anal sphincter (EAS), predisposing to fecal incontinence; normal sphincter profiles for both IAS and EAS; and high IAS and EAS (anismus) predisposing to constipation. C: Percent prevalence of abnormal balloon sensation tests (in red) denoting impaired rectal sensation. D: Percent prevalence of absent recto-anal inhibitory reflex (in red), suggestive of impaired recto-anal coordination. The bottom panel shows a pie chart highlighting the prevalence of defecatory dyssynergia types (I-IV) in the cohort studied by HRAM.

The HRAM characteristics are as follows: (A) the percentage of abnormal balloon expulsion tests (88%, 58 patients), and (B) the percent prevalence of certain anal sphincter measurements, such as low internal anal sphincter (IAS) and low external anal sphincter (EAS) profiles, predisposing to fecal incontinence (48%, 32 patients); normal sphincter profiles for both IAS and EAS (47%, 31 patients); and high IAS and EAS (anismus) predisposing to constipation (5%, 3 patients). Further, they depict (C) the percentage of abnormal balloon sensation tests denoting impaired rectal sensation (30%, 20 patients), and (D) the percentage of patients with absent recto-anal inhibitory reflex, suggestive of impaired recto-anal coordination (18%, 12 patients).

Classification of dyssynergia: Of the 66 patients with chronic constipation, 9 (14%) had normal defecatory coordination, based on HRAM. Except for one, they were also noted to have normal BET. Among the remaining 57 with dyssynergia, type II dyssynergia was the most common (n=27, 41%), followed by type IV (n=26, 39%), followed by type III (n=4, 6%). There was no relationship between HRAM abnormalities and the Hoehn and Yahr scores or disease duration.

### Colonic Transit Time:

Of the 66 patients, 4 could not swallow the WMC, 4 had technical issues not allowing computing of the regional transit times, and 4 could not get insurance authorization. Therefore, only 53 underwent WMC. Of these, 20 (38%) had normal colonic transit time (CTT) with median time of 43 hours (95% CI: 38.32, 45.00). In 33 (62% of the cohort) with prolonged transit, the median CTT was 84.5 hours (95% CI: 71.99. 87.00). Overall, 38 (57% of the total) exhibited overlapping features of dyssynergia and slow transit constipation. There was no correlation between Hoehn and Yahr scores or disease duration and CTT. These results are not surprising since constipation in PD might be an “early” manifestation due to increased α-synuclein in the ENS, and the patients were selected based on severe GI dysfunction NOT stage of disease. Chronic constipation in patients with PD may reflect pelvic floor dyssynergia, slow transit constipation, or both, and may be associated with fecal incontinence, suggesting complex autonomic dysfunction.

The study reveals that the majority (89%) have defecatory dyssynergia (mainly types II and IV), 62% have slow transit constipation and 57% have overlap constipation. These results have therapeutic implications, since different targeted therapies can be applied, alone or in combination, for each patient. The pathophysiology of chronic constipation in PD is complex and difficult to decipher without the use of specialized tests like the ones used. One mechanism, as shown herein as well as in other studies, is prolonged CTT. Another is defecatory dyssynergia, or paradoxical contraction of the striated anal sphincter muscles and/or puborectalis during defecation, and associated, depending on the type (I-IV), with normal or weak rectal contraction; in the study this seems to be the most prevalent abnormality. The data also suggest that poor rectal sensation and lacking recto-anal inhibitory reflex, both suggestive of ENS neuropathy, may be a role in up to 30% of cases, frequently co-existing with anorectal motor dystonia and slow transit constipation. It remains unclear whether rectal hyposensitivity is causative or secondary to neurological or biomechanical dysfunction and its clinical impact still needs to be defined. Constipation may reflect an adverse effect of drugs used in PD. The co-existence of symptoms, such as infrequent evacuation, straining, and a sense of incomplete rectal emptying, suggest that more than one mechanism may be involved. Hence, targeting therapy to specific abnormalities might be more successful. In the study, for example, PD patients with slow transit constipation were treated with osmotic laxatives, lubiprostone or linaclotide, those with dyssynergia were treated with biofeedback and pelvic floor exercises, while many patients were treated with both modalities. Such therapies were not formally assessed as part of the study given its retrospective nature and the lack of standardization of the endpoints for each therapy.

In the retrospective study patients may require multiple interventions (i.e. prucalopride for slow transit constipation, BoTox injection of the anal sphincter for anismus, or loperamide for fecal incontinence, among others). Furthermore, proper instruments to accurately assess the therapeutic response (or lack thereof) in the PD population need to be developed. In general, dietary fiber is not well-tolerated by patients with slow transit constipation and is unlikely to be useful in most patients with PD, and since rectal sensation is frequently preserved, bulking with fiber—aimed at improving rectal sensation—may not be needed. Osmotic laxatives, linaclotide, lubiprostone, and particularly prucalopride, could be useful in the treatment of slow transit constipation, since they shorten CTT. PD patients with absent recto-anal inhibitory reflex (RAIR) may need programmed defecation. On the other hand, biofeedback therapy should be tried in patients with PD who are so often troubled by defecatory dyssynergia, but its efficacy and practical utility is unknown. Since many PD patients with constipation have low anal sphincter pressures, the occurrence of fecal incontinence may be the limiting variable in the overall management.

### Example 6: Measuring Gastric and Small Bowel Abnormalities in PD Patients

For purposes of illustrating this approach in some embodiments, the utility of WMC and lactulose breath tests (LBT) in defining the underlying etiology of symptoms was determined in patients with PD and GI symptoms. Assessment using radiologic and endoscopic tools is important in ruling out structural abnormalities, but these tests are frequently negative or inconclusive. Recent guidelines have supported the use of the WMC for the evaluation of colonic transit in chronic constipation, a common symptom in patients with PD. This new and non-invasive technology has allowed quantification of transit in all gut regions in a single test and it has been increasingly used for the diagnosis of functional GI symptoms, such as bloating, early postprandial fullness and nausea, among others. Small intestinal bacterial overgrowth (SIBO), defined as the presence of excessive bacteria in the small bowel may cause nonspecific symptoms, such as bloating, abdominal distension or discomfort, diarrhea, and weight loss. These symptoms likely reflect not only bacterial overgrowth-induced mucosal inflammation but also the underlying cause, such as dysmotility and delayed small bowel transit. Recently, the role of GI microbiota in PD pathogenesis has received attention and some phenotypic correlations have been shown. LBT is a widely-used method for diagnosis of SIBO and, if positive, allows for antimicrobial therapy aiming at bacterial eradication and symptom relief. Used together, WMC and LBT are poised to guide optimal therapy for GI disorders in PD.

Of the total 95 PD patients, 65 are included in the analysis below. Of the 30 not included, 24 declined testing for various reasons (i.e. not interested, too ill, insurance non-authorization), while 4 patients could not swallow the WMC despite multiple attempts. The mean age of the 65 patients in the cohort was 72 years (range 52-91 years), and 31 (47%) of them were women. The median Hoehn and Yahr score was 3 (95% median CI: 2.72, 3.00); median duration of their PD was 6.5 years (range 0.6-22). The mean scores for the individual symptoms assessed by questionnaires: abdominal pain 0.42, regurgitation 0.45, bloating 1.21, nausea 0.28, vomiting 0.05, belching 0.57, and weight loss 0.53. As compared to all others, bloating was the most significant symptom (p<0.001). The percent prevalence of each individual symptom in the cohort was: abdominal pain 26%; regurgitation 36%; bloating 61%; nausea 17%; vomiting 4%; belching 41%; and weight loss 27%.

The various symptom scores in patients with normal or prolonged Gastric Emptying Time (GET-gastroparesis) (n=64 because in one patient GET could not be computed) and in those with normal or delayed Small Bowel Transit Times (SBTT) (n=60 because in 5 patients SBTT could not be computed) were evaluated. The only significant difference seen was with bloating scores between patients with normal and delayed SBTT (p<0.001). In general, in this group of patients with severe GI symptoms, the symptoms were not discriminatory and could not predict the underlying motor abnormality. There was no relationship between GET and the Hoehn and Yahr scores (Pearson correlation 0.065; p=0.60) or disease duration (Pearson correlation −0.03; p=0.78). Similarly, there was no relationship between SBTT and the Hoehn and Yahr scores (Pearson correlation 0.18; p=0.16) or disease duration (Pearson correlation 0.19; p=0.13).

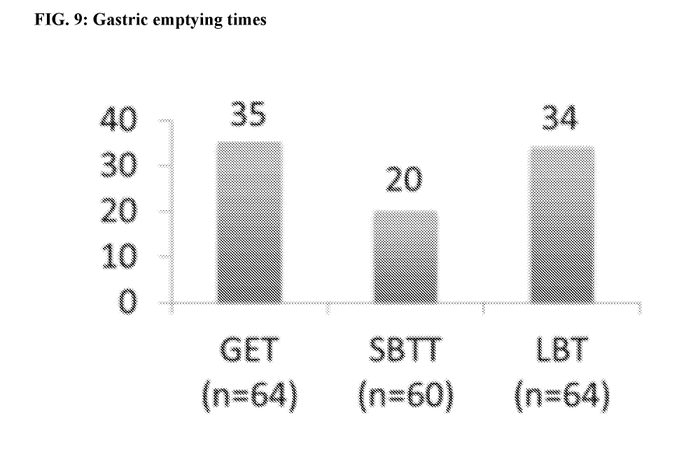

FIG. 9 shows a representative bar graph highlighting the percentages of abnormal gastric emptying time (GET), small bowel transit time (SBTT) and lactose breath test (LBT). The graph shows that 35% exhibited gastroparesis by WMC study, 20% small bowel transit delay, and 8% had combined transit abnormalities, suggesting overlapping gastric and small bowel dysmotility. Thirty-four percent of the 64 patients studied (one had technically inadequate study) had positive LBT, suggestive of small bowel overgrowth. In the patients with prolonged GET, the mean GET was 12.6±1.5 hours; in those with prolonged SBTT the mean was 10.4±1.4 hours. There was no relationship between LBT, Hoehn and Yahr scores or disease duration.

Any clinical utility in performing WMC and LBT in patients with PD with various functional GI symptoms was determined. A significant percentage of the patients (35%) have GET, 20% delayed SBTT, and 8% overlapping gastric and small bowel transit delay. Thirty-four percent of such patients have SIBO. Accordingly, hese results have therapeutic implications, since different targeted therapies can be applied, alone or in combination, for each patient. One mechanism, as shown herein as well as in other studies, is prolonged GTT (gastroparesis). Another is delayed SBTT which, based on the data, contributes significantly to the clinically distressing symptom of bloating. This has not been previously examined, but it could reflect deposition of α-synuclein or myenteric plexus neuronal loss.

Relevance to Care and implications for early diagnosis: Unintended weight loss was common in patients with PD and correlated with impaired quality of life (QOL); in the study, 26% had moderate to severe weight loss. Malnutrition in PD is linked to reduced food intake not only because of loss of appetite but also because of early fullness and pain after meals, bloating, and nausea as seen in the study and others. Underlying SIBO, as it was shown in 34% of patients in the study, may play an important role. Treatment with antibiotics such as rifaximin is indicated, as recent studies have shown improvement in motor fluctuations following bacterial eradication. An additional factor to be considered, given the findings of delayed gastric and small bowel motility, is the poor pharmacokinetics of drugs used in PD treatment, a concern raised in previous studies but could not be addressed in this retrospective study.

Implications for early diagnosis and measures of disease progression: data from these analyses could serve as preliminary elements and a launching pad in further understanding of the relationship between the disease and its gut manifestations. The performance of WMC and LBT led in many patients not only to a specific diagnosis but also therapy theretofore unavailable (i.e. BoTox injection of the pyloric sphincter, oral rifaximin). Functional GI symptoms are prevalent in patients with PD and may reflect underlying gastroparesis, small bowel transit delay, or both, and may be associated with SIBO.

Clinically relevant endpoints for assessment comprise time of onset of GI symptoms in relation to the diagnosis of PD motor symptoms, to characterize the timing of onset of GI symptoms in relation to the clinical diagnosis of PD.

Clinically relevant endpoints for assessment comprise the mean change from baseline to years 1, 2 and 3 in GSRS total and subscores for abdominal pain, reflux, indigestion, diarrhea, and constipation to assess changes in GI symptoms as measured by the GSRS. Though the total score is used for analysis, the subscores that target specific GI symptoms in patients with PD is also used in the analysis. Subscore analysis of specific GI symptoms on the GSRS is consistent with analysis of the GSRS in other randomized clinical trials.

Clinically relevant endpoints for assessment comprise the mean change from baseline to years 1, 2 and 3 in GCSI total and subscores for nausea/vomiting, post-prandial fullness/early satiety, and bloating to assess changes in GI symptoms as measured by the GCSI. Though the total score is used for analysis, the subscores that target specific GI symptoms in patients with PD is also used in the analysis. Subscore analysis of specific GI symptoms on the GCSI is consistent with analysis of the GCSI in other randomized clinical trials.

Clinically relevant endpoints for assessment comprise the mean change from baseline to years 1, 2 and 3 in esophageal motility and sphincter tone measurement, including the key metrics of integrated relaxation pressure, distal contractile integral, distal latency, and contractile front velocity, assessed by HRM using the Chicago classification v3.

Clinically relevant endpoints for assessment comprise the mean change from baseline to years 1, 2 and 3 in anorectal motility parameters (anal sphincter function, rectoanal reflex activity, rectal sensation, changes in anal and rectal pressures during attempted defecation, rectal compliance, and performance of balloon expulsion test using high resolution anorectal manometry).

Clinically relevant endpoints for assessment comprise the mean change from baseline to years 1, 2 and 3 in gastric, small bowel, and colonic transit times measured by SmartPill.

Clinically relevant endpoints for assessment comprise the mean change from baseline to years 1, 2 and 3 in PD medication requirements (number of medications by type).

Clinically relevant endpoints for assessment comprise the mean change from baseline to years 1, 2 and 3 in GI symptom medication requirements (number of medications by type).

Clinically relevant endpoints for assessment comprise Hoehn and Yahr stage, UPDRS Motor Part III and correlation between GSRS and GCSI scales.

Clinically relevant endpoints for assessment comprise the correlation between the number of PD medications (by type) and GI symptoms on the GSRS and GCSI; correlation between the number of GI medications and deficits in manometry and SmartPill; correlation between changes in the types of GI medications and changes in GI symptoms; or any combination thereof.

Clinically relevant endpoints for assessment comprise correlation of the extent of GI disease burden symptoms (e.g. GI symptoms, GI medication usage, and Hoehn and Yahr stage) with the levels of α-synuclein and degree of neuronal loss in GI biopsy and surgical resection samples.

Clinically relevant endpoints for assessment comprise regression model of GI therapies on disease progression/improvement.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### REFERENCES

1. Greenfield, J. G. & Bosanquet, F. D. The Brain-stem lesions in Parkinsonism. J. Neurol. Neurosurg. Psychiatry 16, 213-226 (1953).
2. Cotzias, G. C. L-dopa for Parkinsonism. N. Engl J. Med. 278, 630 (1968).
3. Langston, J. W., Ballard, P., Tetrud, J. W. & Irwin, I. Chronic Parkinsonism in humans due to a product of meperidine-analog synthesis. Science 219, 979-980 (1983).
4. Polymeropoulos, M. H. et al. Mutation in the alpha-synuclein gene identified in families with Parkinson's disease. Science 276, 2045-2047 (1997).
5. Singleton, A. B., Farrer, M. J. & Bonifati, V. The genetics of Parkinson's disease: progress and therapeutic implications. Mov. Disord. 28, 14-23 (2013).
6. Langston, J. W. The Parkinson's complex: parkinsonism is just the tip of the iceberg. Ann. Neurol. 59, 591-596 (2006).
7. Meissner, W. G. When does Parkinson's disease begin? From prodromal disease to motor signs. Rev. Neurol. (Paris) 168, 809-814 (2012).
8. Lewy, F. H. Paralysis Agitans. I. Pathologische Anatomic (Springer, Berlin, 1912).
9. Herzog, E. Histopathologische Veränderungen im Sympathicus and ihre Bedeutung. Dtsch. Z. Nervenheilkd. 107, 75-80 (1928).
10. Braak, H., Ghebremedhin, E., Rub, U., Bratzke, H. & Del Tredici, K. Stages in the development of Parkinson's disease-related pathology. Cell Tissue Res. 318, 121-134 (2004).
11. Braak, H. et al. Staging of the intracerebral inclusion body pathology associated with idiopathic Parkinson's disease (preclinical and clinical stages). J. Neurol. 249 (Suppl. 3), III1-III5 (2002).
12. Savica, R., Rocca, W. A. & Ahlskog, J. E. When does Parkinson disease start? Arch. Neurol. 67, 798-801 (2010).
13. Del Tredici, K., Rub, U., De Vos, R. A., Bohl, J. R. & Braak, H. Where does parkinson disease pathology begin in the Brain? J. Neuropathol. Exp. Neurol. 61, 413-426 (2002).
14. Kosaka, K., Yoshimura, M., Ikeda, K. & Budka, H. Diffuse type of Lewy body disease: progressive dementia with abundant cortical Lewy bodies and senile changes of varying degree—a new disease? Clin. Neuropathol. 3, 185-192 (1984).
15. Hishikawa, N., Hashizume, Y., Yoshida, M. & Sobue, G. Clinical and neuropathological correlates of Lewy body disease. Acta Neuropathol. 105, 341-350 (2003).
16. Goldstein, D. S. Cardiac denervation in patients with Parkinson disease. Cleve. Clin. J. Med. 74 (Suppl. 1), S91-S94 (2007).
17. Gelpi, E. et al. Multiple organ involvement by alpha-synuclein pathology in Lewy body disorders. Mov. Disord. 29, 1010-1018 (2014).
18. Klein, C. & Westenberger, A. Genetics of Parkinson's disease. Cold Spring Harb. Perspect. Med. 2, a008888 (2012).
19. Nalls, M. A. et al. Large-scale meta-analysis of genome-wide association data identifies six new risk loci for Parkinson's disease. Nat. Genet. 46, 989-993 (2014).
20. Richards, C. S. et al. ACMG recommendations for standards for interpretation and reporting of sequence variations: revisions 2007. Genet. Med. 10, 294-300 (2008).
21. Iritani, S., Tsuchiya, K., Arai, T., Akiyama, H. & Ikeda, K. An atypical autopsy case of Lewy body disease with clinically diagnosed major depression: a clinical, radiological and pathological study. Neuropathology 28, 652-659 (2008).
22. Kalia, L. V. et al. Clinical correlations with Lewy body pathology in LRRK2-related Parkinson disease. JAMA Neurol. 72, 100-105 (2015).
23. Quattrone, A. et al. Myocardial 123metaiodobenzylguanidine uptake in genetic Parkinson's disease. Mov. Disord. 23, 21-27 (2008).
24. Chen, Y. et al. Quantitative and fiber-selective evaluation of pain and sensory dysfunc-tion in patients with Parkinson's disease. Parkinsonism Relat. Disord. 21, 361-365 (2015).
25. Hamza, T. H. et al. Genome-wide gene-environment study identifies glutamate receptor gene GRIN2A as a Parkinson's disease modifier gene via interaction with coffee. PLoS Genet. 7, e1002237 (2011).
26. Goldman, S. M. et al. Genetic modification of the association of paraquat and Parkinson's disease. Mov. Disord. 27, 1652-1658 (2012).
27. Langston, J. W., Langston, E. B. & Irwin, I. MPTP-induced parkinsonism in human and non-human primates-clinical and experimental aspects. Acta Neurol. Scand. Suppl. 100, 49-54 (1984).
28. Langston, J. W., Quik, M., Petzinger, G., Jakowec, M. & Di Monte, D. A. Investigating levodopa-induced dyskinesias in the parkinsonian primate. Ann. Neurol. 47, S79-S89 (2000).
29. Schüle, B., Pera, R. A. & Langston, J. W. Can cellular models revolutionize drug discovery in Parkinson's disease? Biochim. Biophys. Acta 1792, 1043-1051 (2009).
30. Byers, B. et al. SNCA triplication Parkinson's patient's iPSC-derived DA Neurons accu-mulate a-synuclein and are susceptible to oxidative stress. PLoS ONE 6, e26159 (2011).
31. Nguyen, H. N. et al. LRRK2 mutant iPSC-derived DA Neurons demonstrate increased susceptibility to oxidative stress. Cell Stem Cell 8, 267-280 (2011).
32. Flierl, A. et al. Higher vulnerability and stress sensitivity of Neuronal precursor cells carrying an alpha-synuclein gene triplication. PLoS ONE 9, e112413 (2014).
33. Reinhardt, P. et al. Genetic correction of a LRRK2 mutation in human iPSCs links parkinsonian neurodegeneration to ERK-dependent changes in gene expression. Cell Stem Cell 12, 354-367 (2013).
34. Chung, C. Y. et al. Identification and rescue of alpha-synuclein toxicity in Parkinson patient-derived Neurons. Science 342, 983-987 (2013).
35. Soldner, F. et al. Parkinson's disease patient-derived induced pluripotent stem cells free of viral reprogramming factors. Cell 136, 964-977 (2009).
36. Seibler, P. et al. Mitochondrial Parkin recruitment is impaired in Neurons derived from mutant PINK1 induced pluripotent stem cells. J. Neurosci. 31, 5970-5976 (2011).
37. Sánchez-Danés, A. et al. Disease-specific phenotypes in dopamine Neurons from human iPS-based models of genetic and sporadic Parkinson's disease. EMBO Mol. Med. 4, 380-395 (2012).
38. Liu, G. H. et al. Progressive degeneration of human neural stem cells caused by patho-genic LRRK2. Nature 491, 603-607 (2012).
39. Reyes, J. F. et al. A cell culture model for monitoring alpha-synuclein cell-to-cell transfer. Neurobiol. Dis. 77, 266-275 (2015).
40. Aboud, A. A. et al. Genetic risk for Parkinson's disease correlates with alterations in Neuronal manganese sensitivity between two human subjects. Neurotoxicology 33, 1443-1449 (2012).
41. Chan, P. et al. Absence of mutations in the coding region of the alpha-synuclein gene in pathologically proven Parkinson's disease. Neurology 50, 1136-1137 (1998).
42. Chan, P., Tanner, C. M., Jiang, X. & Langston, J. W. Failure to find the alpha-synuclein gene missense mutation (G209A) in 100 patients with younger onset Parkinson's disease. Neurology 50, 513-514 (1998).
43. Farrer, M. et al. Comparison of kindreds with parkinsonism and alpha-synuclein genomic multiplications. Ann. Neurol. 55, 174-179 (2004).
44. Tetrud, J. W. & Langston, J. W. The effect of deprenyl (selegiline) on the natural history of Parkinson's disease. Science 245, 519-522 (1989).
45. Plasterer, T. N., Stanley, R. & Gombocz, E. Correlation Network Analysis and Knowledge Integration (Wiley-VCH, Weinheim, 2011).
46. Lynge, E., Sandegaard, J. L. & Rebolj, M. The Danish National Patient Register. Scand. J. Public Health 39, 30-33 (2011).
47. Nilsson, E., Orwelius, L. & Kristenson, M. Patient-reported outcomes in the Swedish National Quality Registers. J. Intern. Med. doi:10.1111/joim.12409 (26 Aug. 2015).
48. Jensen, L. J. et al. STRING 8—a global view on proteins and their functional interactions in 630 organisms. Nucleic Acids Res. 37, D412-D416 (2009).
49. Mrowka, R., Patzak, A. & Herzel, H. Is there a bias in proteome research? Genome Res. 11, 1971-1973 (2001).
50. Chouraki, V. & Seshadri, S. Genetics of Alzheimer's disease. Adv. Genet. 87, 245-294 (2014).
51. Seidel, K. et al. First appraisal of Brain pathology owing to A30P mutant alpha-synu-clein. Ann. Neurol. 67, 684-689 (2010).
52. Zarranz, J. J. et al. The new mutation, E46K, of alpha-synuclein causes Parkinson and Lewy body dementia. Ann. Neurol. 55, 164-173 (2004).
53. Proukakis, C. et al. A novel alpha-synuclein missense mutation in Parkinson disease. Neurology 80, 1062-1064 (2013).
54. Kiely, A. P. et al. a-Synucleinopathy associated with G51D SNCA mutation: a link between Parkinson's disease and multiple system atrophy? Acta Neuropathol. 125, 753-769 (2013).
55. Kiely, A. P. et al. Distinct clinical and neuropathological features of G51D SNCA mutation cases compared with SNCA duplication and H50Q mutation. Mol. Neurodegener. 10, 41 (2015).
56. Lesage, S. et al. G51D alpha-synuclein mutation causes a novel parkinsonian-pyramidal syndrome. Ann. Neurol. 73, 459-471 (2013).
57. Golbe, L. I., Di Iorio, G., Bonavita, V., Miller, D. C. & Duvoisin, R. C. A large kindred with autosomal dominant Parkinson's disease. Ann. Neurol. 27, 276-282 (1990).
58. Duda, J. E. et al. Concurrence of alpha-synuclein and tau Brain pathology in the Contursi kindred. Acta Neuropathol. 104, 7-11 (2002).
59. Spira, P. J., Sharpe, D. M., Halliday, G., Cavanagh, J. & Nicholson, G. A. Clinical and pathological features of a Parkinsonian syndrome in a family with an Ala53Thr alpha-synuclein mutation. Ann. Neurol. 49, 313-319 (2001).
60. Markopoulou, K. et al. Clinical, neuropathological and genotypic variability in SNCA A53T familial Parkinson's disease. Variability in familial Parkinson's disease. Acta Neuropathol. 116, 25-35 (2008).
61. Yamaguchi, K. et al. Abundant neuritic inclusions and microvacuolar changes in a case of diffuse Lewy body disease with the A53T mutation in the alpha-synuclein gene. Acta Neuropathol. 110, 298-305 (2005).
62. Kasten, M. & Klein, C. The many faces of alpha-synuclein mutations. Mov. Disord. 28, 697-701 (2013).
63. Garraux, G. et al. Partial trisomy 4q associated with young-onset dopa-responsive parkinsonism. Arch. Neurol. 69, 398-400 (2012).
64. Nishioka, K. et al. Expanding the clinical phenotype of SNCA duplication carriers. Mov. Disord. 24, 1811-1819 (2009).
65. Kara, E. et al. A 6.4 Mb duplication of the alpha-synuclein locus causing frontotem-poral dementia and Parkinsonism: phenotype-genotype correlations. JAMA Neurol. 71, 1162-1171 (2014).
66. Konno, T., Ross, O. A., Puschmann, A., Dickson, D. W. & Wszolek, Z. K. Autosomal dominant Parkinson's disease caused by SNCA duplications. Parkinsonism Relat. Disord. doi:10.1016/j.parkreldis.2015.09.007 (3 Sep. 2015).
67. Obi, T. et al. Clinicopathologic study of a SNCA gene duplication patient with Parkinson disease and dementia. Neurology 70, 238-241 (2008).
68. Ikeuchi, T. et al. Patients homozygous and heterozygous for SNCA duplication in a family with parkinsonism and dementia. Arch. Neurol. 65, 514-519 (2008).
69. Waters, C. H. & Miller, C. A. Autosomal dominant Lewy body parkinsonism in a four-generation family. Ann. Neurol. 35, 59-64 (1994).
70. Muenter, M. D. et al. Hereditary form of parkinsonism—dementia. Ann. Neurol. 43, 768-781 (1998).
71. Gwinn-Hardy, K. et al. Distinctive Neuropathology revealed by alpha-synuclein antibodies in hereditary parkinsonism and dementia linked to chromosome 4p. Acta Neuropathol. 99, 663-672 (2000).
72. Giordana, M. T. et al. Neuropathology of Parkinson's disease associated with the LRRK2 Ile1371Val mutation. Mov. Disord. 22, 275-278 (2007).
73. Puschmann, A. et al. First neuropathological description of a patient with Parkinson's disease and LRRK2 p.N1437H mutation. Parkinsonism Relat. Disord. 18, 332-338 (2012).
74. Martí-Massó J. F. et al. Neuropathology of Parkinson's disease with the R1441G mutation in LRRK2. Mov. Disord. 24, 1998-2001 (2009).
75. Wszolek, Z. K. et al. Western Nebraska family (family D) with autosomal dominant parkinsonism. Neurology 45, 502-505 (1995).
76. Wszolek, Z. K. et al. Autosomal dominant parkinsonism associated with variable synu-clein and tau pathology. Neurology 62, 1619-1622 (2004).
77. Khan, N. L. et al. Mutations in the gene LRRK2 encoding dardarin (PARK8) cause familial Parkinson's disease: clinical, pathological, olfactory and functional imaging and genetic data. Brain 128, 2786-2796 (2005).
78. Ross, O. A. et al. Lrrk2 and Lewy body disease. Ann. Neurol. 59, 388-393 (2006).
79. Gomez, A. & Ferrer, I. Involvement of the cerebral cortex in Parkinson disease linked with G2019S LRRK2 mutation without cognitive impairment. Acta Neuropathol. 120, 155-167 (2010).
80. Silveira-Moriyama, L. et al. Hyposmia in G2019S LRRK2-related parkinsonism: clinical and pathologic data. Neurology 71, 1021-1026 (2008).
81. Gilks, W. P. et al. A common LRRK2 mutation in idiopathic Parkinson's disease. Lancet 365, 415-416 (2005).
82. Hasegawa, K. et al. Familial parkinsonism: study of original Sagamihara PARK8 (I2020T) kindred with variable clinicopathologic outcomes. Parkinsonism Relat. Disord. 15, 300-306 (2009).
83. Hasegawa, K. & Kowa, H. Autosomal dominant familial Parkinson disease: older onset of age, and good response to levodopa therapy. Eur. Neurol. 38 Suppl 1, 39-43 (1997).
84. Chahine, L. M. et al. Clinical and biochemical differences in patients having Parkinson disease with vs without GBA mutations. JAMA Neurol. 70, 852-858 (2013).
85. Sidransky, E. & Lopez, G. The link between the GBA gene and parkinsonism. Lancet Neurol. 11, 986-998 (2012).
86. Neumann, J. et al. Glucocerebrosidase mutations in clinical and pathologically proven Parkinson's disease. Brain 132, 1783-1794 (2009).
87. Poulopoulos, M., Levy, O. A. & Alcalay, R. N. The Neuropathology of genetic Parkinson's disease. Mov. Disord. 27, 831-842 (2012).
88. Langston, J. W. et al. Evidence of active nerve cell degeneration in the substantia nigra of humans years after 1-methyl-4-phenyl-1,2,3,6-tetrahydropyridine exposure. Ann. Neurol. 46, 598-605 (1999).
89. Ahn, T. B., Langston, J. W., Aachi, V. R. & Dickson, D. W. Relationship of neighboring tissue and gliosis to alpha-synuclein pathology in a fetal transplant for Parkinson's disease. Am. J. Neurodegener. Dis. 1, 49-59 (2012).
90. Koga, S. et al. When DLB, PD, and PSP masquerade as MSA: an autopsy study of 134 patients. Neurology 85, 404-412 (2015).
91. Fuchs, J. et al. Phenotypic variation in a large Swedish pedigree due to SNCA duplication and triplication. Neurology 68, 916-922 (2007).
92. Langston, J. W. et al. Novel alpha-synuclein-immunoreactive proteins in Brain samples from the Contursi kindred, Parkinson's, and Alzheimer's disease. Exp. Neurol. 154, 684-690 (1998).
93. Mak, S. K., Tewari, D., Tetrud, J. W., Langston, J. W. & Schüle, B. Mitochondrial dysfunction in skin fibroblasts from a Parkinson's disease patient with an alpha-synuclein triplication. J. Parkinsons Dis. 1, 175-183 (2011).
94. Marras, C. et al. Phenotype in parkinsonian and nonparkinsonian LRRK2 G2019S mutation carriers. Neurology 77, 325-333 (2011).
95. Sanders, L. H. et al. LRRK2 mutations cause mitochondrial DNA damage in iPSC-derived neural cells from Parkinson's disease patients: reversal by gene correction. Neurobiol. Dis. 62, 381-386 (2014).
96. Lwin, A., Orvisky, E., Goker-Alpan, O., LaMarca, M. E. & Sidransky, E. Glucocerebrosidase mutations in subjects with parkinsonism. Mol. Genet. Metab. 81, 70-73 (2004).
97. Farrer, M. et al. Lewy bodies and parkinsonism in families with parkin mutations. Ann. Neurol. 50, 293-300 (2001).
98. Schüle, B., Byrne, C., Rees, L. & Langston, J. W. Is PARKIN parkinsonism a cancer predisposition syndrome? Neurol. Genet. 1, e31 (15 Oct. 2015).
99. Doostzadeh, J., Tetrud, J. W., Allen-Auerbach, M., Langston, J. W. & Schüle, B. Novel features in a patient homozygous for the L347P mutation in the PINK1 gene. Parkinsonism Relat. Disord. 13, 359-361 (2007).

### REFERENCES FOR TABLE 1 AND FIG. 10

1. Krüger, R. et al. Ala30Pro mutation in the gene encoding alpha-synuclein in Parkinson's disease. Nat Genet 18, 106-8 (1998).
2. Seidel, K. et al. First appraisal of brain pathology owing to A30P mutant alpha-synuclein. Ann Neurol 67, 684-9 (2010).
3. Zarranz, J. J. et al. The new mutation, E46K, of alpha-synuclein causes Parkinson and Lewy body dementia. Ann Neurol 55, 164-73 (2004).
4. Appel-Cresswell, S. et al. Alpha-synuclein p.H50Q, a novel pathogenic mutation for Parkinson's disease. Mov Disord 28, 811-3 (2013).
5. Proukakis, C. et al. A novel alpha-synuclein missense mutation in Parkinson disease. Neurology 80, 1062-4 (2013).
6. Kiely, A. P. et al. alpha-Synucleinopathy associated with G51D SNCA mutation: a link between Parkinson's disease and multiple system atrophy? Acta Neuropathol 125, 753-69 (2013).
7. Tokutake, T. et al. Clinical and neuroimaging features of patient with early-onset Parkinson's disease with dementia carrying SNCA p.G51D mutation. Parkinsonism Relat Disord 20, 262-4 (2014).
8. Kiely, A. P. et al. Distinct clinical and neuropathological features of G51D SNCA mutation cases compared with SNCA duplication and H50Q mutation. Mol Neurodegener 10, 41 (2015).
9. Lesage, S. et al. G51D alpha-synuclein mutation causes a novel parkinsonian-pyramidal syndrome. Ann Neurol 73, 459-71 (2013).
10. Polymeropoulos, M. H. et al. Mutation in the alpha-synuclein gene identified in families with Parkinson's disease. Science 276, 2045-7 (1997).
11. Golbe, L. I., Di Iorio, G., Bonavita, V., Miller, D. C. & Duvoisin, R. C. A large kindred with autosomal dominant Parkinson's disease. Ann Neurol 27, 276-82 (1990).
12. Duda, J. E. et al. Concurrence of alpha-synuclein and tau brain pathology in the Contursi kindred. Acta Neuropathol 104, 7-11 (2002).
13. Spira, P. J., Sharpe, D. M., Halliday, G., Cavanagh, J. & Nicholson, G. A. Clinical and pathological features of a Parkinsonian syndrome in a family with an Ala53Thr alpha-synuclein mutation. Ann Neurol 49, 313-9 (2001).
14. Yamaguchi, K. et al. Abundant neuritic inclusions and microvacuolar changes in a case of diffuse Lewy body disease with the A53T mutation in the alpha-synuclein gene. Acta Neuropathol 110, 298-305 (2005).
15. Markopoulou, K. et al. Clinical, neuropathological and genotypic variability in SNCA A53T familial Parkinson's disease. Variability in familial Parkinson's disease. Acta Neuropathol 116, 25-35 (2008).
16. Konno, T., Ross, O. A., Puschmann, A., Dickson, D. W. & Wszolek, Z. K. Autosomal dominant Parkinson's disease caused by SNCA duplications. Parkinsonism Relat Disord (2015).
17. Obi, T. et al. Clinicopathologic study of a SNCA gene duplication patient with Parkinson disease and dementia. Neurology 70, 238-41 (2008).
18. Chartier-Harlin, M. C. et al. Alpha-synuclein locus duplication as a cause of familial Parkinson's disease. Lancet 364, 1167-9 (2004).
19. Kara, E. et al. A 6.4 Mb Duplication of the alpha-Synuclein Locus Causing Frontotemporal Dementia and Parkinsonism: Phenotype-Genotype Correlations. JAMA Neurol 71, 1162-71 (2014).
20. Ikeuchi, T. et al. Patients homozygous and heterozygous for SNCA duplication in a family with parkinsonism and dementia. Arch Neurol 65, 514-9 (2008).
21. Fuchs, J. et al. Phenotypic variation in a large Swedish pedigree due to SNCA duplication and triplication. Neurology 68, 916-22 (2007).
22. Singleton, A. B. et al. alpha-Synuclein locus triplication causes Parkinson's disease. Science 302, 841 (2003).
23. Sekine, T. et al. Clinical course of the first Asian family with Parkinsonism related to SNCA triplication. Mov Disord 25, 2871-5 (2010).
24. Ibanez, P. et al. Alpha-synuclein gene rearrangements in dominantly inherited parkinsonism: frequency, phenotype, and mechanisms. Arch Neurol 66, 102-8 (2009).
25. Garraux, G. et al. Partial trisomy 4q associated with young-onset dopa-responsive parkinsonism. Arch Neurol 69, 398-400 (2012).
26. Kasten, M. & Klein, C. The many faces of alpha-synuclein mutations. Mov Disord 28, 697-701 (2013).
27. Poulopoulos, M., Levy, O. A. & Alcalay, R. N. The Neuropathology of genetic Parkinson's disease. Mov Disord 27, 831-42 (2012).
28. Tijero, B. et al. Cardiac sympathetic denervation in symptomatic and asymptomatic carriers of the E46K mutation in the alpha synuclein gene. Parkinsonism Relat Disord 19, 95-100 (2013).
29. Tijero, B. et al. Cardiac sympathetic denervation precedes nigrostriatal loss in the E46K mutation of the alpha-synuclein gene (SNCA). Clin Auton Res 20, 267-9 (2010).
30. Orimo, S. et al. Cardiac sympathetic denervation in Parkinson's disease linked to SNCA duplication. Acta Neuropathol 116, 575-7 (2008).
31. Singleton, A. et al. Association between cardiac denervation and parkinsonism caused by alpha-synuclein gene triplication. Brain 127, 768-72 (2004).
32. Aasly, J. O. et al. Novel pathogenic LRRK2 p.Asn1437His substitution in familial Parkinson's disease. Mov Disord 25, 2156-63 (2010).
33. Zimprich, A. et al. Mutations in LRRK2 cause autosomal-dominant parkinsonism with pleomorphic pathology. Neuron 44, 601-7 (2004).
34. Paisan-Ruiz, C. et al. Cloning of the gene containing mutations that cause PARKS-linked Parkinson's disease. Neuron 44, 595-600 (2004).
35. Tan, E. K. et al. LRRK2 R1628P increases risk of Parkinson's disease: replication evidence. Hum Genet 124, 287-8 (2008).
36. Ross, O. A. et al. Analysis of Lrrk2 R1628P as a risk factor for Parkinson's disease. Ann Neurol 64, 88-92 (2008).
37. Ozelius, L. J. et al. LRRK2 G2019S as a cause of Parkinson's disease in Ashkenazi Jews. N Engl J Med 354, 424-5 (2006).
38. Farrer, M. J. et al. Lrrk2 G2385R is an ancestral risk factor for Parkinson's disease in Asia. Parkinsonism Relat Disord 13, 89-92 (2007).
39. Tan, E. K. Identification of a common genetic risk variant (LRRK2 Gly2385Arg) in Parkinson's disease. Ann Acad Med Singapore 35, 840-2 (2006).
40. Kruger, R. LRRK2 in Parkinson's disease—drawing the curtain of penetrance: a commentary. BMC Med 6, 33 (2008).
41. Latourelle, J. C. et al. The Gly2019Ser mutation in LRRK2 is not fully penetrant in familial Parkinson's disease: the GenePD study. BMC Med 6, 32 (2008).
42. Healy, D. G. et al. Phenotype, genotype, and worldwide genetic penetrance of LRRK2-associated Parkinson's disease: a case-control study. Lancet Neurol 7, 583-90 (2008).
43. Kalia, L. V. et al. Clinical correlations with Lewy body pathology in LRRK2-related Parkinson's disease. JAMA Neurology (in press).
44. Ross, O. A. et al. Lrrk2 and Lewy body disease. Ann Neurol 59, 388-93 (2006).
45. Gomez, A. & Ferrer, I. Involvement of the cerebral cortex in Parkinson disease linked with G2019S LRRK2 mutation without cognitive impairment. Acta Neuropathol 120, 155-67 (2010).
46. Silveira-Moriyama, L. et al. Hyposmia in G2019S LRRK2-related parkinsonism: clinical and pathologic data. Neurology 71, 1021-6 (2008).
47. Gilks, W. P. et al. A common LRRK2 mutation in idiopathic Parkinson's disease. Lancet 365, 415-6 (2005).
48. Quattrone, A. et al. Myocardial 123metaiodobenzylguanidine uptake in genetic Parkinson's disease. Mov Disord 23, 21-7 (2008).
49. Pastores, G. M. & Hughes, D. A. Gaucher Disease in GeneReviews® 1993-2015 edn Vol. 2015 (eds Pagon, R. A., Adam, M. P. & Ardinger, H. H.) (University of Washington, Seattle, 2000 Jul. 27).
50. Chahine, L. M. et al. Clinical and biochemical differences in patients having Parkinson disease with vs without GBA mutations. JAMA Neurol 70, 852-8 (2013).
51. Sidransky, E. & Lopez, G. The link between the GBA gene and parkinsonism. Lancet Neurol 11, 986-98 (2012).
52. Lwin, A., Orvisky, E., Goker-Alpan, O., LaMarca, M. E. & Sidransky, E. Glucocerebrosidase mutations in subjects with parkinsonism. Mol Genet Metab 81, 70-3 (2004).
53. Goker-Alpan, O. et al. The spectrum of parkinsonian manifestations associated with glucocerebrosidase mutations. Arch Neurol 65, 1353-7 (2008).
54. Neumann, J. et al. Glucocerebrosidase mutations in clinical and pathologically proven Parkinson's disease. Brain 132, 1783-94 (2009).
55. Valente, E. M. et al. Localization of a novel locus for autosomal recessive early-onset parkinsonism, PARK6, on human chromosome 1p35-p36. Am J Hum Genet 68, 895-1900 (2001).
56. Ibanez, P. et al. Mutational analysis of the PINK1 gene in early-onset parkinsonism in Europe and North Africa. Brain 129, 686-94 (2006).
57. Hatano, Y. et al. Novel PINK1 mutations in early-onset parkinsonism. Ann Neurol 56, 424-7 (2004).
58. Valente, E. M. et al. PINK1 mutations are associated with sporadic early-onset parkinsonism. Ann Neurol 56, 336-41 (2004).
59. Takao, M. et al. Spinocerebellar ataxia type 2 is associated with Parkinsonism and Lewy body pathology. BMJ Case Rep 2011 (2011).
60. Vilarino-Guell, C. et al. DNAJC13 mutations in Parkinson disease. Hum Mol Genet 23, 1794-801 (2014).
61. Appel-Cresswell, S. et al. Clinical, positron emission tomography, and pathological studies of DNAJC13 p.N855S Parkinsonism. Mov Disord 29, 1684-7 (2014).
62. Rajput, A. et al. VPS35 and DNAJC13 disease-causing variants in essential tremor. Eur J Hum Genet (2014).
63. Kitada, T. et al. Mutations in the parkin gene cause autosomal recessive juvenile parkinsonism. Nature 392, 605-8 (1998).
64. Lücking, C. B. et al. Association between early-onset Parkinson's disease and mutations in the parkin gene. N Engl J Med 342, 1560-7 (2000).
65. Bonifati, V. Autosomal recessive parkinsonism. Parkinsonism Relat Disord 18 Suppl 1, S4-6 (2012).
66. Miyakawa, S. et al. Lewy body pathology in a patient with a homozygous parkin deletion. Mov Disord 28, 388-91 (2013).
67. Pramstaller, P. P. et al. Lewy body Parkinson's disease in a large pedigree with 77 Parkin mutation carriers. Ann Neurol 58, 411-22 (2005).
68. Tijero, B. et al. Autonomic involvement in Parkinsonian carriers of PARK2 gene mutations. Parkinsonism Relat Disord 21, 717-22 (2015).
69. Sasaki, S., Shirata, A., Yamane, K. & Iwata, M. Parkin-positive autosomal recessive juvenile Parkinsonism with alpha-synuclein-positive inclusions. Neurology 63, 678-82 (2004).
70. Gouider-Khouja, N. et al. Autosomal recessive parkinsonism linked to parkin gene in a Tunisian family. Clinical, genetic and pathological study. Parkinsonism Relat Disord 9, 247-51 (2003).
71. Orimo, S. et al. Preserved cardiac sympathetic nerve accounts for normal cardiac uptake of MIBG in PARK2. Mov Disord 20, 1350-3 (2005).
72. Mori, H., Hattori, N. & Mizuno, Y. Genotype-phenotype correlation: familial Parkinson disease. Neuropathology 23, 90-4 (2003).
73. Cornejo-Olivas, M. R. et al. A Peruvian family with a novel PARK2 mutation: Clinical and pathological characteristics. Parkinsonism Relat Disord 21, 444-8 (2015).
74. van de Warrenburg, B. P. et al. Clinical and pathologic abnormalities in a family with parkinsonism and parkin gene mutations. Neurology 56, 555-7 (2001).
75. Hayashi, S. et al. An autopsy case of autosomal-recessive juvenile parkinsonism with a homozygous exon 4 deletion in the parkin gene. Mov Disord 15, 884-8 (2000).
76. Yamamura, Y. et al. Clinical, pathologic and genetic studies on autosomal recessive early-onset parkinsonism with diurnal fluctuation. Parkinsonism Relat Disord 4, 65¬72 (1998).
77. Mori, H. et al. Pathologic and biochemical studies of juvenile parkinsonism linked to chromosome 6q. Neurology 51, 890-2 (1998).
78. Doherty, K. M. et al. Parkin disease: a clinicopathologic entity? JAMA Neurol 70, 571-9 (2013).
79. Ruffmann, C. et al. Lewy body pathology and typical Parkinson disease in a patient with a heterozygous (R275W) mutation in the Parkin gene (PARK2). Acta Neuropathol 123, 901-3 (2012).
80. Sharp, M. E. et al. Parkinson's disease with Lewy bodies associated with a heterozygous PARKIN dosage mutation. Mov Disord 29, 566-8 (2014).
81. Sanchez, M. P., Gonzalo, I., Avila, J. & De Yebenes, J. G. Progressive supranuclear palsy and tau hyperphosphorylation in a patient with a C212Y parkin mutation. J Alzheimers Dis 4, 399-404 (2002).
82. Zimprich, A. et al. A mutation in VPS35, encoding a subunit of the retromer complex, causes late-onset Parkinson disease. Am J Hum Genet 89, 168-75 (2011).
83. Vilarino-Guell, C. et al. VPS35 mutations in Parkinson disease. Am J Hum Genet 89, 162-7 (2011).
84. Struhal, W. et al. VPS35 Parkinson's disease phenotype resembles the sporadic disease. J Neural Transm 121, 755-9 (2014).
85. Kumar, K. R. et al. Frequency of the D620N mutation in VPS35 in Parkinson disease. Arch Neurol 69, 1360-4 (2012).
86. Sharma, M. et al. A multi-centre clinico-genetic analysis of the VPS35 gene in Parkinson disease indicates reduced penetrance for disease-associated variants. J Med Genet 49, 721-6 (2012).
87. Wider, C. et al. Autosomal dominant dopa-responsive parkinsonism in a multigenerational Swiss family. Parkinsonism Relat Disord 14, 465-70 (2008).
88. Bonifati, V. et al. Mutations in the DJ-1 gene associated with autosomal recessive early-onset parkinsonism. Science 299, 256-9 (2003).
89. Macedo, M. G. et al. Genotypic and phenotypic characteristics of Dutch patients with early onset Parkinson's disease. Mov Disord 24, 196-203 (2009).
90. Ohta, E. et al. Novel mutations in the guanosine triphosphate cyclohydrolase 1 gene associated with DYT5 dystonia. Arch Neurol 63, 1605-10 (2006).
91. Tadic, V. et al. Dopa-responsive dystonia revisited: diagnostic delay, residual signs, and nonmotor signs. Arch Neurol 69, 1558-62 (2012).
92. Mencacci, N. E. et al. Parkinson's disease in GTP cyclohydrolase 1 mutation carriers. Brain 137, 2480-92 (2014).
93. Furukawa, Y. & Kish, S. J. Dopa-responsive dystonia: recent advances and remaining issues to be addressed. Mov Disord 14, 709-15 (1999).
94. Guella, I. et al. Parkinsonism in GTP cyclohydrolase 1 mutation carriers. Brain (2014).
95. Yamashita, C. et al. Evaluation of polyglutamine repeats in autosomal dominant Parkinson's disease. Neurobiol Aging 35, 1779 e17-21 (2014).
96. Kim, J. M. et al. Importance of low-range CAG expansion and CAA interruption in SCA2 Parkinsonism. Arch Neurol 64, 1510-8 (2007).
97. Lu, C. S., Wu Chou, Y. H., Kuo, P. C., Chang, H. C. & Weng, Y. H. The parkinsonian phenotype of spinocerebellar ataxia type 2. Arch Neurol 61, 35-8 (2004).
98. Payami, H. et al. SCA2 may present as levodopa-responsive parkinsonism. Mov Disord 18, 425-9 (2003).
99. Rub, U. et al. Clinical features, neurogenetics and Neuropathology of the polyglutamine spinocerebellar ataxias type 1, 2, 3, 6 and 7. Prog Neurobiol 104, 38-66 (2013).
100. Socal, M. P. et al. Intrafamilial variability of Parkinson phenotype in SCAs: novel cases due to SCA2 and SCA3 expansions. Parkinsonism Relat Disord 15, 374-8 (2009).
101. Yomono, H. S. et al. [Autopsy case of SCA2 with Parkinsonian phenotype]. Rinsho Shinkeigaku 50, 156-62 (2010).
102. De Rosa, A. et al. Reduced cardiac 123I-metaiodobenzylguanidine uptake in patients with spinocerebellar ataxia type 2: a comparative study with Parkinson's disease. Eur J Nucl Med Mol Imaging 40, 1914-21 (2013).
103. Butcher, N. J. et al. Association between early-onset Parkinson disease and 22q11.2 deletion syndrome: identification of a novel genetic form of Parkinson disease and its clinical implications. JAMA Neurol 70, 1359-66 (2013).
104. Booij, J., van Amelsvoort, T. & Boot, E. Co-occurrence of early-onset Parkinson disease and 22q11.2 deletion syndrome: Potential role for dopamine transporter imaging. Am J Med Genet A 152A, 2937-8 (2010).
105. Krahn, L. E., Maraganore, D. M. & Michels, V. V. Childhood-onset schizophrenia associated with parkinsonism in a patient with a microdeletion of chromosome 22. Mayo Clin Proc 73, 956-9 (1998).
106. Zaleski, C. et al. The co-occurrence of early onset Parkinson disease and 22q11.2 deletion syndrome. Am J Med Genet A 149A, 525-8 (2009).
107. Hartig, M. B. et al. Absence of an orphan mitochondrial protein, c19orf12, causes a distinct clinical subtype of neurodegeneration with Brain iron accumulation. Am J Hum Genet 89, 543-50 (2011).
108. Dogu, O. et al. Rapid disease progression in adult-onset mitochondrial membrane protein-associated neurodegeneration. Clin Genet 84, 350-5 (2013).
109. Hogarth, P. et al. New NBIA subtype: genetic, clinical, pathologic, and radiographic features of MPAN. Neurology 80, 268-75 (2013).
110. Landoure, G. et al. Hereditary spastic paraplegia type 43 (SPG43) is caused by mutation in C19orf12. Hum Mutat 34, 1357-60 (2013).
111. Morgan, N. V. et al. PLA2G6, encoding a phospholipase A2, is mutated in neurodegenerative disorders with high Brain iron. Nat Genet 38, 752-4 (2006).
112. Paisan-Ruiz, C. et al. Characterization of PLA2G6 as a locus for dystonia-parkinsonism. Ann Neurol 65, 19-23 (2009).
113. Paisan-Ruiz, C. et al. Early-onset L-dopa-responsive parkinsonism with pyramidal signs due to ATP13A2, PLA2G6, FBXO7 and spatacsin mutations. Mov Disord 25, 1791-800 (2010).
114. Paisan-Ruiz, C. et al. Widespread Lewy body and tau accumulation in childhood and adult onset dystonia-parkinsonism cases with PLA2G6 mutations. Neurobiol Aging 33, 814-23 (2012).
115. Sina, F., Shojaee, S., Elahi, E. & Paisan-Ruiz, C. R632W mutation in PLA2G6 segregates with dystonia-parkinsonism in a consanguineous Iranian family. Eur J Neurol 16, 101-4 (2009).
116. Yoshino, H. et al. Phenotypic spectrum of patients with PLA2G6 mutation and PARK14-linked parkinsonism. Neurology 75, 1356-61 (2010).
117. Riku, Y. et al. Extensive aggregation of alpha-synuclein and tau in juvenile-onset neuroaxonal dystrophy: an autopsied individual with a novel mutation in the PLA2G6 gene-splicing site. Acta Neuropathol Commun 1, 12 (2013).
118. Gregory, A. & Hayflick, S. J. Pantothenate Kinase-Associated Neurodegeneration. in GeneReviews® 1993-2015 edn Vol. 2015 (eds Pagon, R. A., Adam, M. P. & Ardinger, H. H.) (University of Washington, Seattle, 2002).
119. Thomas, M., Hayflick, S. J. & Jankovic, J. Clinical heterogeneity of neurodegeneration with Brain iron accumulation (Hallervorden-Spatz syndrome) and pantothenate kinase-associated neurodegeneration. Mov Disord 19, 36-42 (2004).
120. Li, A. et al. Pantothenate kinase-associated neurodegeneration is not a synucleinopathy. Neuropathol Appl Neurobiol (2012).
121. Kruer, M. C. et al. Novel histopathologic findings in molecularly-confirmed pantothenate kinase-associated neurodegeneration. Brain 134, 947-58 (2011).
122. Farrer, M. J. et al. DCTN1 mutations in Perry syndrome. Nat Genet 41, 163-5 (2009).
123. Perry, T. L. et al. Hereditary mental depression and Parkinsonism with taurine deficiency. Arch Neurol 32, 108-13 (1975).
124. Araki, E. et al. A novel DCTN1 mutation with late-onset parkinsonism and frontotemporal atrophy. Mov Disord 29, 1201-4 (2014).
125. Ohshima, S. et al. Autonomic failures in Perry syndrome with DCTN1 mutation. Parkinsonism Relat Disord 16, 612-4 (2010).
126. Tacik, P. et al. Three families with Perry syndrome from distinct parts of the world. Parkinsonism Relat Disord 20, 884-8 (2014).
127. Wider, C. et al. Elucidating the genetics and pathology of Perry syndrome. J Neurol Sci 289, 149-54 (2010).
128. Chung, E. J. et al. Expansion of the clinicopathological and mutational spectrum of Perry syndrome. Parkinsonism Relat Disord 20, 388-93 (2014).
129. Newsway, V. et al. Perry syndrome due to the DCTN1 G71R mutation: a distinctive levodopa responsive disorder with behavioral syndrome, vertical gaze palsy, and respiratory failure. Mov Disord 25, 767-70 (2010).
130. Aji, B. M., Medley, G., O'Driscoll, K., Lamer, A. J. & Alusi, S. H. Perry syndrome: a disorder to consider in the differential diagnosis of Parkinsonism. J Neurol Sci 330, 117-8 (2013).
131. Felicio, A. C. et al. In vivo dopaminergic and serotonergic dysfunction in DCTN1 gene mutation carriers. Mov Disord 29, 1197-201 (2014).
132. Puls, I. et al. Distal spinal and bulbar muscular atrophy caused by dynactin mutation. Ann Neurol 57, 687-94 (2005).
133. Puls, I. et al. Mutant dynactin in motor Neuron disease. Nat Genet 33, 455-6 (2003).
134. Munch, C. et al. Point mutations of the p150 subunit of dynactin (DCTN1) gene in ALS.
Neurology 63, 724-6 (2004).
135. Munch, C. et al. Heterozygous R1101K mutation of the DCTN1 gene in a family with ALS and FTD. Ann Neurol 58, 777-80 (2005).
136. Lee, L. V., Munoz, E. L., Tan, K. T. & Reyes, M. T. Sex linked recessive dystonia parkinsonism of Panay, Philippines (XDP). Mol Pathol 54, 362-8 (2001).
137. Nolte, D., Niemann, S. & Muller, U. Specific sequence changes in multiple transcript system DYT3 are associated with X-linked dystonia parkinsonism. Proc Natl Acad Sci USA 100, 10347-52 (2003).
138. Altrocchi, P. H. & Forno, L. S. Spontaneous oral-facial dyskinesia: Neuropathology of a case. Neurology 33, 802-5 (1983).
139. Waters, C. H. et al. Neuropathology of lubag (x-linked dystonia parkinsonism). Mov Disord 8, 387-90 (1993).
140. Tuite, P. J., Rogaeva, E. A., St George-Hyslop, P. H. & Lang, A. E. Dopa-responsive parkinsonism phenotype of Machado-Joseph disease: confirmation of 14q CAG expansion. Ann Neurol 38, 684-7 (1995).
141. Wang, J. L. et al. Analysis of SCA2 and SCA3/MJD repeats in Parkinson's disease in mainland China: genetic, clinical, and positron emission tomography findings. Mov Disord 24, 2007-11 (2009).
142. Gwinn-Hardy, K. et al. Spinocerebellar ataxia type 3 phenotypically resembling parkinson disease in a black family. Arch Neurol 58, 296-9 (2001).
143. Bettencourt, C. et al. Parkinsonian phenotype in Machado-Joseph disease (MJD/SCA3): a two-case report. BMC Neurol 11, 131 (2011).
144. Lu, C. S. et al. The parkinsonian phenotype of spinocerebellar ataxia type 3 in a Taiwanese family. Parkinsonism Relat Disord 10, 369-73 (2004).
145. Buhmann, C., Bussopulos, A. & Oechsner, M. Dopaminergic response in Parkinsonian phenotype of Machado-Joseph disease. Mov Disord 18, 219-21 (2003).
146. Tokumaru, A. M. et al. Magnetic resonance imaging findings of Machado-Joseph disease: histopathologic correlation. J Comput Assist Tomogr 27, 241-8 (2003).
147. Park, J. S. et al. Pathogenic effects of novel mutations in the P-type ATPase ATP13A2 (PARK9) causing Kufor-Rakeb syndrome, a form of early-onset parkinsonism. Hum Mutat 32, 956-64 (2011).
148. Di Fonzo, A. et al. ATP13A2 missense mutations in juvenile parkinsonism and young onset Parkinson disease. Neurology 68, 1557-62 (2007).
149. Ramirez, A. et al. Hereditary parkinsonism with dementia is caused by mutations in ATP13A2, encoding a lysosomal type 5 P-type ATPase. Nat Genet 38, 1184-91 (2006).
150. Yang, X. & Xu, Y. Mutations in the Gene and Parkinsonism: A Preliminary Review. Biomed Res Int 2014, 371256 (2014).
151. Bras, J., Verloes, A., Schneider, S. A., Mole, S. E. & Guerreiro, R. J. Mutation of the parkinsonism gene ATP13A2 causes Neuronal ceroid-lipofuscinosis. Hum Mol Genet 21, 2646-50 (2012).
152. Shojaee, S. et al. Genome-wide linkage analysis of a Parkinsonian-pyramidal syndrome pedigree by 500 K SNP arrays. Am J Hum Genet 82, 1375-84 (2008).
153. Gunduz, A. et al. FBXO7-R498X mutation: Phenotypic variability from chorea to early onset parkinsonism within a family. Parkinsonism Relat Disord (2014).
154. Yalcin-Cakmakli, G. et al. A new Turkish family with homozygous FBXO7 truncating mutation and juvenile atypical parkinsonism. Parkinsonism Relat Disord (2014).
155. Di Fonzo, A. et al. FBXO7 mutations cause autosomal recessive, early-onset parkinsonian-pyramidal syndrome. Neurology 72, 240-5 (2009).
156. Edvardson, S. et al. A deleterious mutation in DNAJC6 encoding the Neuronal-specific clathrin-uncoating co-chaperone auxilin, is associated with juvenile parkinsonism. PLoS One 7, e36458 (2012).
157. Koroglu, C., Baysal, L., Cetinkaya, M., Karasoy, H. & Tolun, A. DNAJC6 is responsible for juvenile parkinsonism with phenotypic variability. Parkinsonism Relat Disord 19, 320-4 (2013).
158. Krebs, C. E. et al. The Sac1 domain of SYNJ1 identified mutated in a family with early-onset progressive Parkinsonism with generalized seizures. Hum Mutat 34, 1200-7 (2013).
159. Olgiati, S. et al. PARK20 caused by SYNJ1 homozygous Arg258Gln mutation in a new Italian family. Neurogenetics 15, 183-8 (2014).
160. Quadri, M. et al. Mutation in the SYNJ1 gene associated with autosomal recessive, early-onset Parkinsonism. Hum Mutat 34, 1208-15 (2013).
161. Kurian, M. A. et al. Homozygous loss-of-function mutations in the gene encoding the dopamine transporter are associated with infantile parkinsonism-dystonia. J Clin Invest 119, 1595-603 (2009).
162. Kurian, M. A. et al. Clinical and molecular characterisation of hereditary dopamine transporter deficiency syndrome: an observational cohort and experimental study. Lancet Neurol 10, 54-62 (2011).
163. Ng, J. et al. Dopamine transporter deficiency syndrome: phenotypic spectrum from infancy to adulthood. Brain 137, 1107-19 (2014).

### REFERENCES FOR TABLE 3 AND FIG. 12

1. Li, X. et al. Leucine-rich repeat kinase 2 (LRRK2)/PARK8 possesses GTPase activity that is altered in familial Parkinson's disease R1441C/G mutants. J Neurochem 103, 238-47 (2007).
2. Wang, L. et al. The chaperone activity of heat shock protein 90 is critical for maintaining the stability of leucine-rich repeat kinase 2. J Neurosci 28, 3384-91 (2008).
3. Tong, Y. et al. R1441C mutation in LRRK2 impairs dopaminergic neurotransmission in mice. Proc Natl Acad Sci USA 106, 14622-7 (2009).
4. Parisiadou, L. et al. Phosphorylation of ezrin/radixin/moesin proteins by LRRK2 promotes the rearrangement of actin cytoskeleton in Neuronal morphogenesis. J Neurosci 29, 13971-80 (2009).
5. Andres-Mateos, E. et al. Unexpected lack of hypersensitivity in LRRK2 knock-out mice to MPTP (1-methyl-4-phenyl-1,2,3,6-tetrahydropyridine). J Neurosci 29, 15846-50 (2009).
6. Li, Y. et al. Mutant LRRK2(R1441G) BAC transgenic mice recapitulate cardinal features of Parkinson's disease. Nat Neurosci 12, 826-8 (2009).
7. Zhou, H. et al. Developing tTA transgenic rats for inducible and reversible gene expression. Int J Biol Sci 5, 171-81 (2009).
8. Lin, X. et al. Leucine-rich repeat kinase 2 regulates the progression of Neuropathology induced by Parkinson's-disease-related mutant alpha-synuclein. Neuron 64, 807-27 (2009).
9. Melrose, H. L. et al. Impaired dopaminergic neurotransmission and microtubule-associated protein tau alterations in human LRRK2 transgenic mice. Neurobiol Dis 40, 503-17 (2010).
10. Dachsel, J. C. et al. A comparative study of Lrrk2 function in primary Neuronal cultures. Parkinsonism Relat Disord (2010).
11. Li, X. et al. Enhanced striatal dopamine transmission and motor performance with LRRK2 overexpression in mice is eliminated by familial Parkinson's disease mutation G2019S. J Neurosci 30, 1788-97 (2010).
12. Tong, Y. et al. Loss of leucine-rich repeat kinase 2 causes impairment of protein degradation pathways, accumulation of alpha-synuclein, and apoptotic cell death in aged mice. Proc Natl Acad Sci USA 107, 9879-84 (2010).
13. Winner, B. et al. Adult neurogenesis and neurite outgrowth are impaired in LRRK2 G2019S mice. Neurobiol Dis 41, 706-16 (2011).
14. Herzig, M. C. et al. LRRK2 protein levels are determined by kinase function and are crucial for kidney and lung homeostasis in mice. Hum Mol Genet (2011).
15. Zhou, H. et al. Temporal expression of mutant LRRK2 in adult rats impairs dopamine reuptake. Int J Biol Sci 7, 753-61 (2011).
16. Ramonet, D. et al. Dopaminergic Neuronal loss, reduced neurite complexity and autophagic abnormalities in transgenic mice expressing G2019S mutant LRRK2. PLoS One 6, e18568 (2011).
17. Gillardon, F., Schmid, R. & Draheim, H. Parkinson's disease-linked leucine-rich repeat kinase 2(R1441G) mutation increases proinflammatory cytokine release from activated primary microglial cells and resultant neurotoxicity. NeuroScience 208, 41-8 (2012).
18. Friedman, L. G. et al. Disrupted autophagy leads to dopaminergic axon and dendrite degeneration and promotes presynaptic accumulation of alpha-synuclein and LRRK2 in the Brain. J Neurosci 32, 7585-93 (2012).
19. Moehle, M. S. et al. LRRK2 inhibition attenuates microglial inflammatory responses. J Neurosci 32, 1602-11 (2012).
20. Maekawa, T. et al. The I2020T Leucine-rich repeat kinase 2 transgenic mouse exhibits impaired locomotive ability accompanied by dopaminergic Neuron abnormalities. Mol Neurodegener 7, 15 (2012).
21. Daher, J. P. et al. Neurodegenerative phenotypes in an A53T alpha-synuclein transgenic mouse model are independent of LRRK2. Hum Mol Genet 21, 2420-31 (2012).
22. Lee, M. K. et al. Human alpha-synuclein-harboring familial Parkinson's disease-linked Ala-53-->Thr mutation causes neurodegenerative disease with alpha-synuclein aggregation in transgenic mice. Proc Natl Acad Sci USA 99, 8968-73 (2002).
23. Herzig, M. C. et al. High LRRK2 levels fail to induce or exacerbate Neuronal alpha-synucleinopathy in mouse Brain. PLoS One 7, e36581 (2012).
24. Chen, C. Y. et al. (G2019S) LRRK2 activates MKK4-JNK pathway and causes degeneration of SN dopaminergic Neurons in a transgenic mouse model of PD. Cell Death Differ 19, 1623-33 (2012).
25. Hinkle, K. M. et al. LRRK2 knockout mice have an intact dopaminergic system but display alterations in exploratory and motor co-ordination behaviors. Mol Neurodegener 7, 25 (2012).
26. Tong, Y. et al. Loss of leucine-rich repeat kinase 2 causes age-dependent bi-phasic alterations of the autophagy pathway. Mol Neurodegener 7, 2 (2012).
27. Dzamko, N. et al. The IkappaB kinase family phosphorylates the Parkinson's disease kinase LRRK2 at Ser935 and Ser910 during Toll-like receptor signaling. PLoS One 7, e39132 (2012).
28. Paus, M. et al. Enhanced dendritogenesis and axogenesis in hippocampal neuroblasts of LRRK2 knockout mice. Brain Res 1497, 85-100 (2013).
29. Bichler, Z., Lim, H. C., Zeng, L. & Tan, E. K. Non-motor and motor features in LRRK2 transgenic mice. PLoS One 8, e70249 (2013).
30. Dranka, B. P. et al. Diapocynin prevents early Parkinson's disease symptoms in the leucine-rich repeat kinase 2 (LRRK2R(1)(4)(4)(1)G) transgenic mouse. Neurosci Lett 549, 57-62 (2013).
31. Sepulveda, B., Mesias, R., Li, X., Yue, Z. & Benson, D. L. Short- and long-term effects of LRRK2 on axon and dendrite growth. PLoS One 8, e61986 (2013).
32. Bailey, R. M. et al. LRRK2 phosphorylates novel tau epitopes and promotes tauopathy. Acta Neuropathol 126, 809-27 (2013).
33. Santacruz, K. et al. Tau suppression in a neurodegenerative mouse model improves memory function. Science 309, 476-81 (2005).
34. Baptista, M. A. et al. Loss of leucine-rich repeat kinase 2 (LRRK2) in rats leads to progressive abnormal phenotypes in peripheral organs. PLoS One 8, e80705 (2013).
35. Yang, S. et al. Mitochondrial dysfunction driven by the LRRK2-mediated pathway is associated with loss of Purkinje cells and motor coordination deficits in diabetic rat model. Cell Death Dis 5, e1217 (2014).
36. Sanchez, G. et al. Unaltered striatal dopamine release levels in young Parkin knockout, Pink1 knockout, DJ-1 knockout and LRRK2 R1441G transgenic mice. PLoS One 9, e94826 (2014).
37. Miklavc, P. et al. Surfactant secretion in LRRK2 knock-out rats: changes in lamellar body morphology and rate of exocytosis. PLoS One 9, e84926 (2014).
38. Parisiadou, L. et al. LRRK2 regulates synaptogenesis and dopamine receptor activation through modulation of PKA activity. Nat Neurosci 17, 367-76 (2014).
39. Chou, J. S. et al. (G2019S) LRRK2 causes early-phase dysfunction of SNpc dopaminergic Neurons and impairment of corticostriatal long-term depression in the PD transgenic mouse. Neurobiol Dis 68, 190-9 (2014).
40. Caesar, M. et al. Changes in matrix metalloprotease activity and progranulin levels may contribute to the pathophysiological function of mutant leucine-rich repeat kinase 2. Glia 62, 1075-92 (2014).
41. Longo, F., Russo, I., Shimshek, D. R., Greggio, E. & Moran, M. Genetic and pharmacological evidence that G2019S LRRK2 confers a hyperkinetic phenotype, resistant to motor decline associated with aging. Neurobiol Dis 71, 62-73 (2014).
42. Beccano-Kelly, D. A. et al. LRRK2 overexpression alters glutamatergic presynaptic plasticity, striatal dopamine tone, postsynaptic signal transduction, behavioural hypoactivity and memory deficits. Hum Mol Genet (2014).
43. Beccano-Kelly, D. A. et al. Synaptic function is modulated by LRRK2 and glutamate release is increased in cortical Neurons of G2019S LRRK2 knock-in mice. Front Cell Neurosci 8, 301 (2014).
44. Daher, J. P., Volpicelli-Daley, L. A., Blackburn, J. P., Moehle, M. S. & West, A. B. Abrogation of alpha-synuclein-mediated dopaminergic neurodegeneration in LRRK2-deficient rats. Proc Natl Acad Sci USA 111, 9289-94 (2014).
45. Tsika, E. et al. Conditional expression of Parkinson's disease-related R1441C LRRK2 in midBrain dopaminergic Neurons of mice causes nuclear abnormalities without neurodegeneration. Neurobiol Dis 71, 345-58 (2014).
46. Lee, J. W., Tapias, V., Di Maio, R., Greenamyre, J. T. & Cannon, J. R. Behavioral, neurochemical, and pathologic alterations in bacterial artificial chromosome transgenic G2019S leucine-rich repeated kinase 2 rats. Neurobiol Aging 36, 505-18 (2015).

### REFERENCES FOR TABLE 5 AND FIG. 14

1. Takahashi, K. et al. Induction of pluripotent stem cells from adult human fibroblasts by defined factors. Cell 131, 861-72 (2007).
2. Mali, P. et al. Improved efficiency and pace of generating induced pluripotent stem cells from human adult and fetal fibroblasts. Stem Cells 26, 1998-2005 (2008).
3. Maherali, N. et al. A high-efficiency system for the generation and study of human induced pluripotent stem cells. Cell Stem Cell 3, 340-5 (2008).
4. Zhou, W. & Freed, C. R. Adenoviral gene delivery can reprogram human fibroblasts to induced pluripotent stem cells. Stem Cells 27, 2667-74 (2009).
5. Stadtfeld, M., Nagaya, M., Utikal, J., Weir, G. & Hochedlinger, K. Induced Pluripotent Stem Cells Generated Without Viral Integration. Science (2008).
6. Yang, W. et al. iPSC Reprogramming from Human Peripheral Blood Using Sendai Virus Mediated Gene Transfer. in StemBook (Cambridge (Mass.), 2008).
7. Daheron, L. & D'Souza, S. Blood—SeV derived fibroblast generated iPSCs. in StemBook (Cambridge (Mass.), 2008).
8. Yu, J. et al. Human Induced Pluripotent Stem Cells Free of Vector and Transgene Sequences. Science (2009).
9. Hiratsuka, M. et al. Integration-free iPS cells engineered using human artificial chromosome vectors. PLoS One 6, e25961 (2011).
10. Woltjen, K. et al. piggyBac transposition reprograms fibroblasts to induced pluripotent stem cells. Nature (2009).
11. Plews, J. R. et al. Activation of pluripotency genes in human fibroblast cells by a novel mRNA based approach. PLoS One 5, e14397 (2010).
12. Warren, L., Ni, Y., Wang, J. & Guo, X. Feeder-free derivation of human induced pluripotent stem cells with messenger RNA. Sci Rep 2, 657 (2012).
13. Bernal, J. A. RNA-based tools for nuclear reprogramming and lineage-conversion: towards clinical applications. J Cardiovasc Transl Res 6, 956-68 (2013).
14. Rhee, Y. H. et al. Protein-based human iPS cells efficiently generate functional dopamine Neurons and can treat a rat model of Parkinson disease. J Clin Invest 121, 2326-35 (2011).
15. Dick, E. et al. Two new protocols to enhance the production and isolation of human induced pluripotent stem cell lines. Stem Cell Res 6, 158-67 (2011).
16. Dick, E., Matsa, E., Young, L. E., Darling, D. & Denning, C. Faster generation of hiPSCs by coupling high-titer lentivirus and column-based positive selection. Nat Protoc 6, 701-14 (2011).
17. Hubbard, J. J. et al. Efficient iPS Cell Generation from Blood Using Episomes and HDAC Inhibitors. J Vis Exp (2014).
18. Nishishita, N. et al. Generation of virus-free induced pluripotent stem cell clones on a synthetic matrix via a single cell subcloning in the naive state. PLoS One 7, e38389 (2012).
19. Rodin, S. et al. Clonal culturing of human embryonic stem cells on laminin-521/E-cadherin matrix in defined and xeno-free environment. Nat Commun 5, 3195 (2014).
20. Kawasaki, H. et al. Induction of midBrain dopaminergic Neurons from ES cells by stromal cell-derived inducing activity. Neuron 28, 31-40 (2000).
21. Yue, F., Cui, L., Johkura, K., Ogiwara, N. & Sasaki, K. Induction of midBrain dopaminergic Neurons from primate embryonic stem cells by coculture with sertoli cells. Stem Cells 24, 1695-706 (2006).
22. Swistowski, A. et al. Xeno-free defined conditions for culture of human embryonic stem cells, neural stem cells and dopaminergic Neurons derived from them. PLoS One 4, e6233 (2009).
23. Swistowski, A. et al. Efficient generation of functional dopaminergic Neurons from human induced pluripotent stem cells under defined conditions. Stem Cells 28, 1893-904 (2010).
24. Nori, S. et al. Grafted human-induced pluripotent stem-cell-derived neurospheres promote motor functional recovery after spinal cord injury in mice. Proc Natl Acad Sci USA 108, 16825-30 (2011).
25. Kriks, S. et al. Dopamine Neurons derived from human ES cells efficiently engraft in animal models of Parkinson's disease. Nature (2011).
26. Chambers, S. M. et al. Highly efficient neural conversion of human ES and iPS cells by dual inhibition of SMAD signaling. Nat Biotechnol 27, 275-80 (2009).
27. Chambers, S. M., Mica, Y., Studer, L. & Tomishima, M. J. Converting human pluripotent stem cells to neural tissue and Neurons to model neurodegeneration. Methods Mol Biol 793, 87-97 (2011).
28. Mak, S. K. et al. Small molecules greatly improve conversion of human induced pluripotent stem cells to the Neuronal lineage. Stem Cells International (2012).
29. Theka, I. et al. Rapid generation of functional dopaminergic Neurons from human induced pluripotent stem cells through a single-step procedure using cell lineage transcription factors. Stem Cells Transl Med 2, 473-9 (2013).

What is claimed is:

1.-74. (canceled)

75. A method of early diagnosis of MLBD in a subject, comprising:
(a) performing an assessment of a subject's peripheral autonomic system dysfunction;
(b) assigning a quantitative score to the assessment of the subject's peripheral autonomic system dysfunction based on prevalence of the dysfunction in MLBD patients;
(c) comparing the quantitative score to a predetermined range indicative of risk of developing MLBD; and
(d) identifying the subject as suffering from or prone to MLBD if the quantitative score falls in the predetermined range.

76. The method of claim 75, further comprising assessing one or more of the following factors of the subject:
(a) motor symptoms;
(b) mutation in one or more genes selected from the group consisting of: LRRK2, GBA, and SNCA;
(c) neuropathology; or
any combination thereof; and
assigning a quantitative score for the assessed factors based on prevalence in MLBD patients.

77. The method of claim 75, wherein the peripheral autonomic system dysfunction comprises cardiac denervation or gastrointestinal (GI) dysfunction.

78. The method of claim 75, further comprising administering a neuroprotective agent to treat the subject whose quantitative score is above a threshold as compared to a control.

79. The method of claim 75, further comprising administering a therapeutic agent to treat the subject's GI dysfunction in combination with the neuroprotective agent.

80. The method of claim 75, wherein assessing the subject's GI dysfunction comprises assessing the subject's enteric nervous system for a genetic mutation in one or more of genes selected from the group consisting of: LRRK2, GBA, SNCA, and any combination thereof.

81. The method of claim 75, further comprising assessing the subject's enteric nervous system for presence of alpha-synuclein positive Lewy bodies or Lewy neurites.

82. A method of diagnosing a subject for Parkinson's disease, comprising:
(a) performing an assessment of a gastrointestinal (GI) condition using one or more of the following methods: esophageal manometry, anorectal manometry, wireless motility capsule, GI symptom questionnaires, or any combination thereof;
(b) assigning a quantitative score to the GI condition assessed based on prevalence of the condition in Parkinson's disease patients; and
(c) comparing said quantitative score to a predetermined range indicative of risk of developing Parkinson's disease.

83. The method of claim 82, further comprising administering a neuroprotective agent to the subject whose quantitative score is above a threshold value.

84. The method of claim 82, further comprising obtaining a biopsy of the subject's enteric nervous system and testing said biopsy for a genetic mutation in one or more of genes selected from the group consisting of: LRRK2, GBA, SNCA, and any combination thereof.

85. The method of claim 82, further comprising obtaining a biopsy of the subject's enteric nervous system and testing said biopsy for presence of alpha-synuclein positive Lewy bodies or Lewy neurites.

86.-89. (canceled)

90. A method of diagnosing MLBD, comprising assessing a subject's GI motility using one or more of the following methods: esophageal manometry, anorectal manometry, wireless motility capsule, GI symptom questionnaires, a G-Tech monitoring device, a GI Symptom Relief Scale (GSRS), a Gastroparesis Cardinal Symptom Index (GCSI), a UPSIT, a Hoehn Yahr Scale, a UPDRS scale, or any combination thereof.

91. (canceled)

92. A method of treating MLBD, comprising administering a neuroprotective agent to the subject diagnosed with MLBD using the method of claim 90.

93. The method of claim 92, wherein the therapeutic agent comprises one or more of the following: carbidopa, levodopa, dopamine agonist, MAO-B inhibitor, Catechol-O-methyltransferase (COMT) inhibitor, anticholinergics, amantadine, antibody, and any combination thereof.

94. The method of claim 90, wherein the subject has no motor symptoms of the disease.

95. (canceled)

96. The method of claim 92, wherein the subject has no motor symptoms of the disease.
